(19) United States
(12) Patent Application Publication
(10) Pub. No.: US 2012/0191039 A1
(43) Pub. Date: Jul. 26, 2012

(54) CARRIER LINKED PRAMIPEXOLE PRODRUGS

(75) Inventors: Harald Rau, Dossenheim (DE); Julia Baron, Heidelberg (DE); Ulrich Hersel, Heidelberg (DE)

(73) Assignee: ASCENDIS PHARMA A/S, Hellerup (DK)

(21) Appl. No.: 13/387,979

(22) PCT Filed: Jul. 30, 2010
(86) PCT No.: PCT/EP2010/061162

§ 371 Date: Mar. 6, 2012

(30) Foreign Application Priority Data
Jul. 31, 2009 (EP) 09167028.1
Oct. 6, 2009 (EP) 09172341.1
Dec. 23, 2009 (EP) 09180522.6

(51) Int. Cl. A61M 5/31; A61K 31/428; A61P 25/16; B32B 1/06; A61P 25/18; A61P 25/24; B32B 1/08; C07D 277/82; A61P 25/00

(52) U.S. Cl. 604/92; 548/163; 514/367; 428/402; 428/35.7; 604/191

(57) ABSTRACT

The present invention relates to a carrier linked pramipexole prodrug or a pharmaceutical acceptable salt thereof, wherein pramipexole is bound via a linker to a polymeric carrier. The invention also relates to pharmaceutical compositions comprising said polymeric pramipexole prodrug and their use as medicaments.

The present invention relates to a carrier linked pramipexole prodrug or a pharmaceutical acceptable salt thereof, wherein pramipexole is bound via a linker to a polymeric carrier. The invention also relates to pharmaceutical compositions comprising said polymeric pramipexole prodrug and their use as medicaments.

To enhance physicochemical or pharmacokinetic properties of a drug, such as pramipexole, in vivo, such drug can be conjugated with a carrier. If the drug is transiently bound to a carrier and/or a linker, such systems are commonly assigned as carrier-linked prodrugs. According to the definitions provided by IUPAC (as given under http://www.chem.qmul.ac.uk/iupac.medchem, accessed on Jul. 22, 2009), a carrier-linked prodrug is a prodrug that contains a temporary linkage of a given active substance with a transient carrier group that produces improved physicochemical or pharmacokinetic properties and that can be easily removed in vivo, usually by a hydrolytic cleavage.

The linkers employed in such carrier-linked prodrugs may be transient, meaning that they are non-enzymatically hydrolytically degradable (cleavable) under physiological conditions (aqueous buffer at pH 7.4, 37° C.) with half-lives ranging from, for example, one hour to three months. On the other hand, stable linkages such as employed in connecting moieties and spacer, are typically non-cleavable permanent bonds meaning that the respective spacer or connecting moiety have a half-life of at least six months under physiological conditions (aqueous buffer at pH 7.4, 37° C.).

Suitable carriers are polymers and can either be directly conjugated to the linker or via a non-cleavable spacer.

The employment of prodrugs with a drug being covalently (chemically) bound to a carrier via the respective drugs' functional groups is well known in the pharmaceutical field. For example, the international application PCT/EP 2009/051079 discloses a prodrug comprising a drug linker conjugate D-L. D is a nitrogen containing biologically active moiety, which forms the respective drug after being released from the linker of the prodrug when administered to a patient. The respective drug (D-H) may be a small molecule bioactive agent or a biopolymer such as proteins, polypeptides, oligonucleotides or peptide nucleic acids. The prodrug also comprises the linker L containing a non-biologically active linker moiety L¹, which is attached to a carrier group, optionally with a spacer in between. The respective drug is covalently bound to the linker moiety L¹ by forming an amide bond. However, it is not disclosed that pramipexole can be employed within said prodrug.

Further linker types are described, for example, in WO-A 2004/108070, which relates to branched polymers being useful in extending the in vivo circulating life of biologically active materials. A component A, which may be a leaving group, a functional group, a hydroxy group or a biologically active moiety is attached via a N,N-bis-(2-hydroxyethyl)glycine amide bicine linker, which in turn may be connected with a carrier, for example a poly(ethylene glycol) (PEG) carrier. However, it is not disclosed that pramipexole can be employed as a drug and that the respective drug has to be mandatorily connected with a polymeric carrier.

On the other hand, the use of pramipexole as a medicament or pharmaceutical compositions containing pramipexole, respectively, are also well known in the art. For example, Papadimitriou et al. (Carbohydrate Polymers, 73 (2008), pages 44-54) describe chitosan nanoparticles loaded with pramipexole. Chitosan is a deacetylated form of chitin, which is an abundant polysaccharide. Pramipexole is not chemically bound to the chitosan nanoparticles, but molecular interactions between the drug (pramipexole) and the chitosan matrix due to its microadhesive properties are responsible for obtaining a molecular dispersion of pramipexole within the nanoparticles. Said molecular dispersions can be employed for a controlled oral delivery of pramipexole to a patient in need thereof.

U.S. Pat. No. 7,344,733 discloses a transdermal therapeutic system (TTS) for the use of pramipexole (and ropinirole). The TTS comprises an active-ingredient-impermeable cover layer, a self-adhesive active-ingredient-containing matrix layer and a peel-off protective layer. The self-adhesive matrix layer comprises polymers such as polyacrylate, which in turn contain the active ingredient (pramipexole). However, the active ingredient is not bound to the polymeric matrix. Pramipexole can be released from said TTS over a period of 24 hours.

EP-A 1 797 871 discloses long acting sustained release formulations for the treatment of Parkinsons's disease comprising a dopamine receptor agonist and a pharmaceutically acceptable biodegradable polymer accessoires. One example of a dopamine receptor agonist is pramipexole, the biodegradable polymer may be poly(lactide-glycolide), poly-(lactic acid) or poly(glycolic acid). The sustained release formulation is prepared by dissolving the dopamine receptor agonist and the pharmaceutically acceptable biodegradable polymer accessoires in an organic solvent. The organic solvent phase is injected into a continuous water phase to form microspheres. After removal of the organic solvent and a filtering step sustained-release microsphere containing the dopamine receptor agonists are obtained. By consequence, the dopamine receptor agonist such as pramipexole is not chemically bound to a polymer within the formulations according to EP-A 1 797 871. Furthermore, the in vitro-in vivo correlation obtained for a long-acting sustained release formulation containing the dopamine receptor agonist rotigotine demonstrates the presence of a burst-type release in beagle dogs that is not visible under in vitro release conditions and therefore raises safety concerns.

Further pharmaceutical formulations containing a pharmaceutical active ingredient such as pramipexole are disclosed in WO 2004/089375, U.S. Pat. No. 7,309,497, US-A 2007/0269482 and WO 03/075887. However, none of said documents discloses formulations, wherein pramipexole is chemically bound to a polymeric carrier.

U.S. Pat. No. 6,927,036 relates to methods for synthesis of 1-(acyloxy)-alkyl derivatives, in particular of such derivatives of pharmacologically effective drugs. Said 1-(acyloxy)-alkyl compounds comprise a NRR¹-fragment, which is defined as a drug such as (among many others) pramipexole. However, the 1-(acyloxy)-alkyl-fragment is not a polymer. The prodrugs of U.S. Pat. No. 6,927,036 can be administered by all methods known in the art such as intramuscular, intravenous or oral, depending on the drug contained in such prodrug and the disease to be treated with. Said prodrugs may in general also be used as a oral sustained release system. U.S. Pat. No. 7,227,028 discloses similar prodrugs based on (acyloxy)-alkyl carbamate.

Parkinson's disease (PD) belongs to a group of conditions called movement disorders. It is characterized by muscle rigidity, resting tremor, a slowing of physical movement and, in extreme cases, a loss of physical movement. The primary symptoms are the results of a degenerative disease of the central nervous system. Insufficient formation and action of dopamine, which is produced in the dopaminergic neurons of the brain located in the substantia nigra causes diminished stimulation of the motor cortex by the basal ganglia. Consequently, dopamine receptor agonists such as ropinirole, pramipexole, pergolide, bromocriptine and piribedil are widely used in the treatment of Parkinson's disease.

Pramipexole is a dopamine receptor agonist with selectivity and specificity for the dopamine D2 subfamily with affinity for the D3 receptors. Pramipexole demonstrated excellent therapeutic efficacy in the treatment of Parkinson's Disease and Restless Legs Syndrome. Pramipexole is rapidly and completely absorbed following oral administration. Oral bioavailability is over 90% and maximum plasma concentration is achieved within 1-2 hours on an empty stomach or 3-4 hours with food although the extent of absorption remains unaffected. Fifteen percent of pramipexole is protein bound and it has a volume of distribution of 7 litres/kg. There is negligible metabolism of pramipexole. Excretion is mainly renal and the elimination half life was found to be 8 hours in healthy subjects. Pramipexole is available as immediate-release tablets in various strengths, designed for oral administration of a single tablet three times per day. Marketed products are, for example, Mirapexin, Mirapex, and Sifrol. See Physicians' Desk Reference 57th edition (2003), 2768-2772.

A three times daily dosing regimen for immediate-release pramipexole dihydrochloride tablets is well tolerated, but patient compliance can be improved through a once-daily oral dosage form. One of the reasons for enhanced patient compliance is that Parkinson's disease is an affliction that becomes more prevalent with advancing age and is often accompanied by decline in memory. A pramipexole once-daily, extended release (ER) formulation is in advanced clinical development. A once-daily regimen demonstrated efficacy and is expected to be particularly useful in enhancing compliance among elderly patients.

In common with other anti-Parkinson's disease drugs, pramipexole has potential to cause undesirable side effects. Side effects of pramipexole have been reported to include orthostatic hypotension, the incidence of which is dose-related. There are also reports of subjects on pramipexole medication experiencing increased somnolence, in particular “sleep attacks”. Such attacks involve a subject falling asleep while engaged in activities of daily living, including operation of a motor vehicle, sometimes resulting in accidents. Development of any long-acting sustained release dosage form of pramipexole must take into account the potential to cause such side effects, so that the new dosage form can be tolerated at least as well as the present tablet formulations.

Dopamine agonist use first-line prevents levodopa-induced motor complications, and especially the ‘priming’ effect. This benefit on dyskinesia has been recently linked with the long elimination half-life that leads to a continuous stimulation of dopaminergic receptors. It has fuelled interest in the notion that it may be better to initiate symptomatic therapy in PD with a relatively long-acting dopamine agonist rather than with a relatively short-acting formulation of levodopa.

Accordingly, there is a need for alternative prodrugs of pramipexole. Such alternative pramipexole prodrugs should be long-acting and characterized by small fluctuation of blood levels. Burst-like fluctuations in blood levels should be avoided, since they are likely to give rise to side effects such as the so-called on-off phenomena. Thus, an object of the present invention is to provide pramipexole prodrugs having a controlled release rate, preferably a sustained release rate.

This object is achieved by a carrier linked pramipexole prodrug or a pharmaceutically acceptable salt thereof, wherein pramipexole is bound via a linker L to a carrier and the carrier is a polymer having a molecular weight of ≧500 g/mol.

It was surprisingly found that such carrier linked prodrugs of pramiprexole containing a polymeric carrier can be used to obtain sustained-release dosage forms of pramipexole which are characterized by a burstless release profile with a low peak-to-trough ratio and strong in vitro-in vivo correlation.

The prodrug according to the present invention show excellent in vivo/in vitro correlation of linker cleavage, a high degree of enzyme independence and show a higher stability at lower pH (pH dependent cleavage).

Within the present invention the terms are used having the meaning as follows.

“Non-biologically active linker” means a linker which does not show the pharmacological effects of the drug pramipexole.

“Alkyl” means a straight-chain or branched carbon chain (unsubstituted alkyl). Optionally, each hydrogen of an alkyl carbon may be replaced by a substituent.

“C₁₋₄ alkyl” means an alkyl chain having 1 to 4 carbon atoms (unsubstituted C₁₋₄ alkyl), e.g. if present at the end of a molecule: methyl, ethyl, n-propyl, isopropyl, n-butyl, isobutyl, sec-butyl tert-butyl, or e.g. —CH₂—, —CH₂—CH₂—, —CH(CH₃)—, —CH₂—CH₂—CH₂—, —CH(C₂H₅)—, —C(CH₃)₂—, when two moieties of a molecule are linked by the alkyl group. Optionally, each hydrogen of a C₁₋₄ alkyl carbon may be replaced by a substituent. Accordingly, “C₁₋₅₀ alkyl” means an alkyl chain having 1 to 50 carbon atoms.

“C₂₋₅₀ alkenyl” means a branched or unbranched alkenyl chain having 2 to 50 carbon atoms (unsubstituted C₂₋₅₀ alkenyl), e.g. if present at the end of a molecule: —CH═CH₂, —CH═CH—CH₃, —CH₂—CH═CH₂, —CH═CH—CH₂—CH₃, —CH═CH—CH═CH₂, or e.g. —CH═CH—, when two moieties of a molecule are linked by the alkenyl group. Optionally, each hydrogen of a C₂₋₅₀ alkenyl carbon may be replaced by a substituent as further specified. Accordingly, the term “alkenyl” relates to a carbon chain with at least one carbon carbon double bond. Optionally, one or more triple bonds may occur.

“C₂₋₅₀ alkynyl” means a branched or unbranched alkynyl chain having 2 to 50 carbon atoms (unsubstituted C₂₋₅₀ alkynyl), e.g. if present at the end of a molecule: —C≡CH, —CH₂—C≡CH, CH₂—CH₂—C≡CH, CH₂—C≡C—CH₃, or e.g. —C≡C— when two moieties of a molecule are linked by the alkynyl group. Optionally, each hydrogen of a C₂₋₅₀ alkynyl carbon may be replaced by a substituent as further specified. Accordingly, the term “alkynyl” relates to a carbon chain with at least one carbon carbon triple bond. Optionally, one or more double bonds may occur.

“C₃₋₇ cycloalkyl” or “C₃₋₇ cycloalkyl ring” means a cyclic alkyl chain having 3 to 7 carbon atoms, which may have carbon-carbon double bonds being at least partially saturated (unsubstituted C₃₋₇ cycloalkyl), e.g. cyclopropyl, cyclobutyl, cyclopentyl, cyclopentenyl, cyclohexyl, cyclohexenyl, cycloheptyl. Optionally, each hydrogen of a cycloalkyl carbon may be replaced by a substituent. The term “C₃₋₇ cycloalkyl” or “C₃₋₇ cycloalkyl ring” also includes bridged bicycles like norbonane (norbonanyl) or norbonene (norbonenyl). Accordingly, “C₃₋₅ cycloalkyl” means a cycloalkyl having 3 to 5 carbon atoms.

“Halogen” means fluoro, chloro, bromo or iodo. It is generally preferred that halogen is fluoro or chloro.

“4 to 7 membered heterocyclyl” or “4 to 7 membered heterocycle” means a ring with 4, 5, 6 or 7 ring atoms that may contain up to the maximum number of double bonds (aromatic or non-aromatic ring which is fully, partially or un-saturated) wherein at least one ring atom up to 4 ring atoms are replaced by a heteroatom selected from the group consisting of sulfur (including —S(O)—, —S(O)₂—), oxygen and nitrogen (including ═N(O)—) and wherein the ring is linked to the rest of the molecule via a carbon or nitrogen atom (unsubstituted 4 to 7 membered heterocyclyl). For the sake of completeness it is indicated that in some embodiments of the present invention, 4 to 7 membered heterocyclyl has to fulfill additional requirements. For example, the linker moiety L¹ of formula (I) contains a fragment X¹, which is defined (among others) as 4 to 7 membered heterocyclyl, with additional requirements in some embodiments. In such a case, if X¹ is 4 to 7 membered heterocyclyl, the respective additional requirements of X¹ have to be considered for 4 to 7 membered heterocyclyl as well. This means that in this case the respective 4 to 7 membered heterocyclyl is incorporated into L¹ via two adjacent ring atoms and the ring atom of said 4 to 7 membered heterocyclyl, which is adjacent to the carbon atom of the amide bond, is also a carbon atom.

Examples for a 4 to 7 membered heterocycles are azetidine, oxetane, thietane, furan, thiophene, pyrrole, pyrroline, imidazole, imidazoline, pyrazole, pyrazoline, oxazole, oxazoline, isoxazole, isoxazoline, thiazole, thiazoline, isothiazole, isothiazoline, thiadiazole, thiadiazoline, tetrahydrofuran, tetrahydrothiophene, pyrrolidine, imidazolidine, pyrazolidine, oxazolidine, isoxazolidine, thiazolidine, isothiazolidine, thiadiazolidine, sulfolane, pyran, dihydropyran, tetrahydropyran, imidazolidine, pyridine, pyridazine, pyrazine, pyrimidine, piperazine, piperidine, morpholine, tetrazole, triazole, triazolidine, tetrazolidine, diazepane, azepine or homopiperazine. Optionally, each hydrogen of a 4 to 7 membered heterocyclyl may be replaced by a substituent.

“9 to 11 membered heterobicyclyl” or “9 to 11 membered heterobicycle” means a heterocyclic system of two rings with 9 to 11 ring atoms, where at least one ring atom is shared by both rings and that may contain up to the maximum number of double bonds (aromatic or non-aromatic ring which is fully, partially or un-saturated) wherein at least one ring atom up to 6 ring atoms are replaced by a heteroatom selected from the group consisting of sulfur (including —S(O)—, —S(O)₂—), oxygen and nitrogen (including ═N(O)—) and wherein the ring is linked to the rest of the molecule via a carbon or nitrogen atom (unsubstituted 9 to 11 membered heterobicyclyl). For the sake of completeness it is indicated that in some embodiments of the present invention, 9 to 11 membered heterobicyclyl has to fulfill additional requirements. For example the linker moiety L¹ of formula (I) contains a fragment X¹, which is defined (among others) as 9 to 11 membered heterobicyclyl, with additional requirements in some embodiments. In such a case, if X¹ is 9 to 11 membered bicyclyl, the respective additional requirements of X¹ have to be considered for 9 to 11 membered heterobicyclyl as well. This means that in this case the respective 9 to 11 membered bicyclyl is incorporated into L¹ via two adjacent ring atoms and the ring atom of said 9 to 11 membered bicyclyl, which is adjacent to the carbon atom of the amide bond, is also a carbon atom.

Examples for a 9 to 11 membered heterobicycle are indole, indoline, benzofuran, benzothiophene, benzoxazole, benzisoxazole, benzothiazole, benzisothiazole, benzimidazole, benzimidazoline, quinoline, quinazoline, dihydroquinazoline, quinoline, dihydroquinoline, tetrahydroquinoline, decahydroquinoline, isoquinoline, decahydroisoquinoline, tetrahydroisoquinoline, dihydroisoquinoline, benzazepine, purine or pteridine. The term 9 to 11 membered heterobicycle also includes spiro structures of two rings like 1,4-dioxa-8-azaspiro[4.5]decane or bridged heterocycles like 8-aza-bicyclo[3.2.1]octane. Optionally, each hydrogen of a 9 to 11 membered heterobicyclyl may be replaced by a substituent.

The term “interrupted” means that between two carbon atoms of, for example, a linker or a spacer or at the respective end of the carbon chain between the respective carbon atom and the hydrogen atom a group (such a —O— or —NH—) is inserted.

In case the prodrugs according to the present invention contain one or more acidic or basic groups, the invention also comprises their corresponding pharmaceutically or toxicologically acceptable salts, in particular their pharmaceutically utilizable salts. Thus, the prodrugs which contain acidic groups can be used according to the invention, for example, as alkali metal salts, alkaline earth metal salts or as ammonium salts. More precise examples of such salts include sodium salts, potassium salts, calcium salts, magnesium salts or salts with ammonia or organic amines such as, for example, ethylamine, ethanolamine, triethanolamine or amino acids. Prodrugs which contain one or more basic groups, i.e. groups which can be protonated, can be present and can be used according to the invention in the form of their addition salts with inorganic or organic acids. Examples for suitable acids include hydrogen chloride, hydrogen bromide, phosphoric acid, sulfuric acid, nitric acid, methanesulfonic acid, p-toluenesulfonic acid, naphthalenedisulfonic acids, oxalic acid, acetic acid, tartaric acid, lactic acid, salicylic acid, benzoic acid, formic acid, propionic acid, pivalic acid, diethylacetic acid, malonic acid, succinic acid, pimelic acid, fumaric acid, maleic acid, malic acid, sulfaminic acid, phenylpropionic acid, gluconic acid, ascorbic acid, isonicotinic acid, citric acid, adipic acid, and other acids known to the person skilled in the art. If the prodrugs simultaneously contain acidic and basic groups in the molecule, the invention also includes, in addition to the salt forms mentioned, inner salts or betaines (zwitterions). The respective salts of the prodrugs of the present invention can be obtained by customary methods which are known to the person skilled in the art like, for example by contacting these with an organic or inorganic acid or base in a solvent or dispersant, or by anion exchange or cation exchange with other salts. The present invention also includes all salts of the prodrugs which, owing to low physiological compatibility, are not directly suitable for use in pharmaceuticals but which can be used, for example, as intermediates for chemical reactions or for the preparation of pharmaceutically acceptable salts.

The term “pharmaceutically acceptable” means approved by a regulatory agency such as the EMEA (Europe) and/or the FDA (US) and/or any other national regulatory agency for use in animals, preferably in humans.

To enhance physicochemical or pharmacokinetic properties of a drug, such as pramipexole, in vivo, such drug can be conjugated with a carrier. If the drug is transiently bound to a carrier and/or a linker, such systems are commonly assigned as carrier-linked prodrugs. According to the definitions provided by IUPAC (as given under http://www.chem.qmul.ac.uk/iupac.medchem, accessed on Jul. 22, 2009), a carrier-linked prodrug is a prodrug that contains a temporary linkage of a given active substance with a transient carrier group that produces improved physicochemical or pharmacokinetic properties and that can be easily removed in vivo, usually by a hydrolytic cleavage.

The linkers employed in such carrier-linked prodrugs are transient, meaning that they are non-enzymatically hydrolytically degradable (cleavable) under physiological conditions (aqueous buffer at pH 7.4, 37° C.) with half-lives ranging from, for example, one hour to three months.

Suitable carriers are polymers and can either be directly conjugated to the linker or via a non-cleavable spacer. The terms “pramipexole hydrogel prodrug” and “hydrogel-linked prodrug of pramipexole” refer to carrier-linked prodrugs of pramipexole, wherein the carrier is a hydrogel and both terms are used synonymously. The terms “hydrogel prodrug” and “hydrogel-linked prodrug” refer to prodrugs of biologically active agents transiently linked to a hydrogel and both terms are used synonymously.

“Pharmaceutical composition” or “composition” means a composition containing one or more active ingredients (for example a drug or a prodrug), and one or more inert ingredients, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a prodrug of the present invention and a pharmaceutically acceptable excipient (pharmaceutically acceptable carrier).

“Dry composition” means that the pramipexole hydrogel prodrug composition is provided in a dry form in a container. Suitable methods for drying are spray-drying and lyophilization (freeze-drying). Such dry composition of pramipexole hydrogel prodrug has a residual water content of a maximum of 10%, preferably less than 5% and more preferably less than 2% (determined according to Karl Fischer). The preferred method of drying is lyophilization. “Lyophilized composition” means that the pramipexole hydrogel polymer prodrug composition was first frozen and subsequently subjected to water reduction by means of reduced pressure. This terminology does not exclude additional drying steps which occur in the manufacturing process prior to filling the composition into the final container.

“Lyophilization” (freeze-drying) is a dehydration process, characterized by freezing a composition and then reducing the surrounding pressure and, optionally, adding heat to allow the frozen water in the composition to sublime directly from the solid phase to gas. Typically, the sublimed water is collected by desublimation.

“Reconstitution” means the restoration of the composition's condition prior to drying, such as a solution or suspension, by adding a liquid prior to administrating the composition to a patient in need thereof. The liquid may contain one or more excipients.

“Reconstitution solution” refers to the liquid used to reconstitute the dry composition of a pramipexole hydrogel prodrug prior to administration to a patient in need thereof.

“Container” means any container in which the pramipexole hydrogel prodrug composition is comprised and can be stored until reconstitution.

“Buffer” or “buffering agent” refers to chemical compounds that maintain the pH in a desired range. Physiologically tolerated buffers are, for example, sodium phosphate, succinate, histidine, bicarbonate, citrate and acetate, sulphate, nitrate, chloride, pyruvate. Antacids such as Mg(OH)₂ or ZnCO₃ may be also used. Buffering capacity may be adjusted to match the conditions most sensitive to pH stability.

“Excipients” refers to compounds administered together with the therapeutic agent, for example, buffering agents, isotonicity modifiers, preservatives, stabilizers, anti-adsorption agents, oxidation protection agents, or other auxiliary agents. However, in some cases, one excipient may have dual or triple functions.

A “lyoprotectant” is a molecule which, when combined with a protein of interest, significantly prevents or reduces chemical and/or physical instability of the protein upon drying in general and especially during lyophilization and subsequent storage. Exemplary lyoprotectants include sugars, such as sucrose or trehalose; amino acids such as arginine, glycine, glutamate or histidine; methylamines such as betaine; lyotropic salts such as magnesium sulfate; polyols such as trihydric or higher sugar alcohols, e.g. glycerin, erythritol, arabitol, xylitol, sorbitol, and mannitol; ethylene glycol; propylene glycol; poly(ethylene glycol); pluronics; hydroxyalkyl starches, e.g. hydroxyethyl starch (HES), and combinations thereof.

“Surfactant” refers to wetting agents that lower the surface tension of a liquid.

“Isotonicity modifiers” refer to compounds which minimize pain that can result from cell damage due to osmotic pressure differences at the injection depot.

The term “stabilizers” refers to compounds used to stabilize the hydrogel prodrug. Stabilisation is achieved by strengthening of the protein-stabilising forces, by destabilisation of the denatured state, or by direct binding of excipients to the protein.

“Anti-adsorption agents” refers to mainly ionic or non-ionic surfactants or other proteins or soluble polymers used to coat or adsorb competitively to the inner surface of the composition's container. Chosen concentration and type of excipient depend on the effect to be avoided but typically a monolayer of surfactant is formed at the interface just above the CMC value.

“Oxidation protection agents” refers to antioxidants such as ascorbic acid, ectoine, glutathione, methionine, monothioglycerol, morin, poly(ethylenimine) (PEI), propyl gallate, vitamin E, chelating agents such as citric acid, EDTA, hexaphosphate, thioglycolic acid.

“Antimicrobial” refers to a chemical substance that kills or inhibits the growth of microorganisms, such as bacteria, fungi, yeasts, protozoans and/or destroys viruses.

“Sealing a container” means that the container is closed in such way that it is airtight, allowing no gas exchange between the outside and the inside and keeping the content sterile.

In the following, the present invention is explained in more detail.

The carrier linked pramipexole prodrugs according to the present invention contain pramipexole. Pramipexole as such is a pharmaceutical (drug) known to a person skilled in the art either in its pure (free base) form or as a pharmaceutically acceptable salt thereof. Therefore, pramipexole is a biologically active drug.

Pramipexole contains two different amino groups. The first amino group is bound to the aromatic (thiazolyl) ring of pramipexole. In the following, this first amino group is denoted with the term “aromatic amino group”, since the aromatic fragment of pramipexole is substituted with said first amino group, which is a primary amino group. The second amino group of pramipexole is bound to the second, non-aromatic ring of pramipexole. By consequence, said second amino group is denoted in the following as “aliphatic amino group”, since the non-aromatic (cyclohexenyl) fragment of pramipexole is substituted with said second amino group. The aliphatic amino group is a secondary amino group, since it contains a propyl substituent.

However, in the present invention the carrier linked pramipexole prodrug or a pharmaceutically acceptable salt thereof does not contain pramipexole in form of its free base or as a pharmaceutically acceptable salt thereof, since pramipexole is bound via a linker L to a carrier. This means that the carrier linked pramipexole prodrugs according to the present invention contain pramipexole as a biologically active moiety. Due to the cleavage of the biological active moiety from the carrier linked pramipexole products when administered to a patient in need thereof, pramipexole is released either in its free form or as a pharmaceutically acceptable salt thereof. In other words, the carrier linked to pramipexole prodrugs contain pramipexole, which is substituted with a linker L, which in turn is bound to a carrier.

Pramipexole may be bound to the linker L via its aromatic amino group to the linker L. More preferably, pramipexole is bound to the linker moiety L¹ as defined below. More preferably, pramipexole is bound to the linker L, in particular to the linker moiety L¹, by forming an amide bond.

The carrier linked pramipexole products according to the present invention contain a carrier, which is bound via the linker L (as defined below) to pramipexole. Preferably, the carrier is bound to the linker moiety L² (as defined below).

The carrier is a polymer having a molecular weight of ≧500 g/mol.

The term polymer describes a molecule comprised of repeating structural units connected by chemical bonds in a linear, circular, branched, crosslinked or dendrimeric way or a combination thereof, which can be of synthetic or biological origin or a combination of both.

Preferred polymers are selected from 2-methacryloyl-oxyethyl phosphoyl cholins, hydrogels, PEG-based hydrogels, poly(acrylic acids), poly(acrylates), poly(acrylamides), poly(alkyloxy)polymers, poly(amides), poly(amidoamines), poly(amino acids), poly(anhydrides), poly(aspartamides), poly(butyric acids), poly(glycolic acids), polybutylene terephthalates, poly(caprolactones), poly(carbonates), poly(cyanoacrylates), poly(dimethylacrylamides), poly(esters), poly(ethylenes), poly(ethylene glycols), poly(ethylene oxides), poly(ethyl phosphates), poly(ethyloxazolines), poly(glycolic acids), poly(hydroxyethyl acrylates), poly(hydroxyethyloxazolines), poly(hydroxymethacrylates), poly(hydroxypropylmethacrylamides), poly(hydroxypropyl methacrylates), poly(hydroxypropyloxazolines), poly(iminocarbonates), poly(lactic acids), poly(lactic-co-glycolic acids), poly(methacrylamides), poly(methacrylates), poly(methyloxazolines), poly(organophosphazenes), poly(ortho esters), poly(oxazolines), polypropylene glycols), poly(siloxanes), poly(urethanes), poly(vinyl alcohols), poly(vinyl amines), poly(vinylmethylethers), poly(vinylpyrrolidones), silicones, celluloses, carbomethyl celluloses, hydroxypropyl methylcelluloses, chitins, chitosans, dextrans, dextrins, gelatins, hyaluronic acids and derivatives, mannans, pectins, rhamnogalacturonans, starches, hydroxyalkyl starches, hydroxyethyl starches and other carbohydrate-based polymers, xylans, and copolymers thereof.

As indicated above, the carrier may be a hydrogel (as one option for a polymer). Hydrogels to be used are known in the art. Suitable hydrogels may be used which are described in WO-A 2006/003014 or EP-A 1 625 856. Accordingly, a hydrogel may be defined as a three-dimensional, hydrophilic or amphiphilic polymeric network capable of taking up large quantities of water. The networks are composed of homopolymers or copolymers, are insoluble due to the presence of covalent chemical or physical (ionic, hydrophobic interactions, entanglements) crosslinks. The crosslinks provide the network structure and physical integrity. Hydrogels exhibit a thermodynamic compatibility with water which allow them to swell in aqueous media. The chains of the network are connected in such a fashion that pores exist and that a substantial fraction of these pores are of dimensions between 1 nm and 1000 nm.

Preferably, the carrier is a biodegradable poly(ethylene glycol) (PEG) based water-insoluble hydrogel. The term “PEG based” as understood herein means that the mass proportion of PEG chains in the hydrogel is at least 10% by weight, preferably at least 25%, based on the total weight of the hydrogel. The remainder can be made up of other spacers and/or oligomers or polymers, such as oligo- or polylysines.

The terms “pramipexole hydrogel prodrug” and “hydrogel-linked pramipexole prodrug” refer to carrier-linked prodrugs of pramipexole, wherein the carrier is a hydrogel and both terms are used synonymously.

“Free form” of a drug refers to the drug in its unmodified, pharmacologically active form, such as after being released from a polymer conjugate.

The terms “drug”, “biologically active molecule”, “biologically active moiety”, “biologically active agent”, “active agent”, and the like mean any substance which can affect any physical or biochemical properties of a biological organism, including but not limited to viruses, bacteria, fungi, plants, animals, and humans. In particular, as used herein, biologically active molecules include any substance intended for diagnosis, cure, mitigation, treatment, or prevention of disease in humans or other animals, or to otherwise enhance physical or mental well-being of humans or animals.

A “therapeutically effective amount” of pramipexole as used herein means an amount sufficient to cure, alleviate or partially arrest the clinical manifestations of a given disease and its complications. An amount adequate to accomplish this is defined as “therapeutically effective amount”. Effective amounts for each purpose will depend on the severity of the disease or injury as well as the weight and general state of the subject. It will be understood that determining an appropriate dosage may be achieved using routine experimentation, by constructing a matrix of values and testing different points in the matrix, which is all within the ordinary skills of a trained physician.

“Stable” and “stability” means that within the indicated storage time the hydrogel conjugates remain conjugated and do not hydrolyze to a substantial extent and exhibit an acceptable impurity profile relating to pramipexole. To be considered stable, the composition contains less than 5% of the drug in its free form.

Moreover the term “water-insoluble” refers to a swellable three-dimensionally crosslinked molecular network forming the hydrogel. The hydrogel if suspended in a large surplus of water or aqueous buffer of physiological osmolality may take up a substantial amount of water, e.g. up to 10-fold on a weight per weight basis, and is therefore swellable but after removing excess water still retains the physical stability of a gel and a shape. Such shape may be of any geometry and it is understood that such an individual hydrogel object is to be considered as a single molecule consisting of components wherein each component is connected to each other component through chemical bonds.

The term “reagent” refers to an intermediate or starting material used in the assembly process leading to a prodrug of the present invention.

The term “chemical functional group” refers to carboxylic acid and activated derivatives, amino, maleimide, thiol and derivatives, sulfonic acid and derivatives, carbonate and derivatives, carbamate and derivatives, hydroxyl, aldehyde, ketone, hydrazine, isocyanate, isothiocyanate, phosphoric acid and derivatives, phosphonic acid and derivatives, haloacetyl, alkyl halides, acryloyl and other alpha-beta unsaturated michael acceptors, arylating agents like aryl fluorides, hydroxylamine, disulfides like pyridyl disulfide, vinyl sulfone, vinyl ketone, diazoalkanes, diazoacetyl compounds, oxirane, and aziridine.

If a chemical functional group is coupled to another chemical functional group, the resulting chemical structure is referred to as “linkage”. For example, the reaction of an amine group with a carboxyl group results in an amide linkage.

“Reactive functional groups” are chemical functional groups of the backbone moiety, which are connected to the hyperbranched moiety.

“Functional group” is the collective term used for “reactive functional group”, “degradable interconnected functional group”, or “conjugate functional group”.

A “degradable interconnected functional group” is a linkage comprising a biodegradable bond which on one side is connected to a spacer moiety connected to a backbone moiety and on the other side is connected to the crosslinking moiety. The terms “degradable interconnected functional group”, “biodegradable interconnected functional group”, “interconnected biodegradable functional group” and “interconnected functional group” are used synonymously.

The terms “blocking group” or “capping group” are used synonymously and refer to moieties which are irreversibly connected to reactive functional groups to render them incapable of reacting with for example chemical functional groups.

The terms “protecting group” or “protective group” refers to a moiety which is reversibly connected to reactive functional groups to render them incapable of reacting with for example other chemical functional groups.

The term “interconnectable functional group” refers to chemical functional groups, which participate in a radical polymerization reaction and are part of the crosslinker reagent or the backbone reagent.

The term “polymerizable functional group” refers to chemical functional groups, which participate in a ligation-type polymerization reaction and are part of the crosslinker reagent and the backbone reagent.

A backbone moiety may comprise a spacer moiety which at one end is connected to the backbone moiety and on the other side to the crosslinking moiety.

The term “derivatives” refers to chemical functional groups suitably substituted with protecting and/or activation groups or to activated forms of a corresponding chemical functional group which are known to the person skilled in the art. For example, activated forms of carboxyl groups include but are not limited to active esters, such as succinimidyl ester, benzotriazyl ester, nitrophenyl ester, pentafluorophenyl ester, azabenzotriazyl ester, acyl halogenides, mixed or symmetrical anhydrides, acyl imidazole.

The term “non-enzymatically cleavable linker” refers to linkers that are hydrolytically degradable under physiological conditions without enzymatic activity.

“Non-biologically active linker” means a linker which does not show the pharmacological effects of the drug (D-H) derived from the biologically active moiety.

The terms “spacer”, “spacer group”, “spacer molecule”, and “spacer moiety” are used interchangeably and if used to describe a moiety present in the hydrogel carrier of the invention, refer to any moiety suitable for connecting two moieties, such as C₁₋₅₀ alkyl, C₂₋₅₀ alkenyl or C₂₋₅₀ alkinyl, which fragment is optionally interrupted by one or more groups selected from —NH—, —N(C₁₋₄ alkyl)-, —O—, —S—, —C(O)—, —C(O)NH—, —C(O)N(C₁₋₄ alkyl)-, —O—C(O)—, —S(O)—, —S(O)₂—, 4 to 7 membered heterocyclyl, phenyl or naphthyl.

The terms “terminal”, “terminus” or “distal end” refer to the position of a functional group or linkage within a molecule or moiety, whereby such functional group may be a chemical functional group and the linkage may be a degradable or permanent linkage, characterized by being located adjacent to or within a linkage between two moieties or at the end of an oligomeric or polymeric chain.

The phrases “in bound form” or “moiety” refer to sub-structures which are part of a larger molecule. The phrase “in bound form” is used to simplify reference to moieties by naming or listing reagents, starting materials or hypothetical starting materials well known in the art, and whereby “in bound form” means that for example one or more hydrogen radicals (—H), or one or more activating or protecting groups present in the reagents or starting materials are not present in the moiety.

It is understood that all reagents and moieties comprising polymeric moieties refer to macromolecular entities known to exhibit variabilities with respect to molecular weight, chain lengths or degree of polymerization, or the number of functional groups. Structures shown for backbone reagents, backbone moieties, crosslinker reagents, and crosslinker moieties are thus only representative examples.

A reagent or moiety may be linear or branched. If the reagent or moiety has two terminal groups, it is referred to as a linear reagent or moiety. If the reagent or moiety has more than two terminal groups, it is considered to be a branched or multi-functional reagent or moiety.

The term “poly(ethylene glycol) based polymeric chain” or “PEG based chain” refers to an oligo- or polymeric molecular chain.

Preferably, such poly(ethylene glycol) based polymeric chain is connected to a branching core, it is a linear poly(ethylene glycol) chain, of which one terminus is connected to the branching core and the other to a hyperbranched dendritic moiety. It is understood that a PEG-based chain may be terminated or interrupted by alkyl or aryl groups optionally substituted with heteroatoms and chemical functional groups.

If the term “poly(ethylene glycol) based polymeric chain” is used in reference to a crosslinker reagent, it refers to a crosslinker moiety or chain comprising at least 20 weight % ethylene glycol moieties.

According to this invention, the hydrogel carrier may be composed of backbone moieties interconnected by hydrolytically degradable bonds.

Preferably, the backbone moiety has a molecular weight in the range of from 1 kDa to 20 kDa, more preferably from 1 kDa to 15 kDa and even more preferably from 1 kDa to 10 kDa. The backbone moieties are preferably also PEG-based comprising one or more PEG chains.

Another aspect of the present invention is a carrier-linked pramipexole prodrug comprising a biodegradable hydrogel of the present invention as carrier, wherein a number of permanent linkages of the backbone moieties exist with a transient prodrug linker to which a biologically active moiety is covalently attached.

The reactive functional groups of a reactive biodegradable hydrogel or modified reactive biodegradable hydrogel serve as attachment points for direct linkage through the before mentioned permanent linkages of pramipexole or pramipexole-linker conjugate. Ideally, the hydrogel-connected drug-linker conjugates are dispersed homogeneously throughout the hydrogel according to the invention, and may or may not be present on the surface of the hydrogel according to the invention.

The functional groups may be attached to a linear chain. In this case, the functional groups may be spaced regularly or irregularly across the chain, or alternatively, the chain may be terminated by two dendritic moieties, providing for the total of functional groups.

Remaining reactive functional groups which are not connected to a transient prodrug linker or to a spacer connected to a transient prodrug linker may be capped with suitable blocking reagents.

Preferably, the covalent attachment formed between the reactive functional groups provided by the backbone moieties and the prodrug linker are permanent bonds. Suitable functional groups for attachment of the prodrug linker to the hydrogel according to the invention include but are not limited to carboxylic acid and derivatives, carbonate and derivatives, hydroxyl, hydrazine, hydroxylamine, maleamic acid and derivatives, ketone, amino, aldehyde, thiol and disulfide.

According to this invention, the biodegradable hydrogel according to the invention is composed of backbone moieties interconnected by hydrolytically degradable bonds.

In a hydrogel carrying drug-linker conjugates according to the invention, a backbone moiety is characterized by a number of functional groups, comprising interconnected biodegradable functional groups and hydrogel-connected drug-linker conjugates, and optionally capping groups. This means that a backbone moiety is characterized by a number of hydrogel-connected drug-linker conjugates; functional groups, comprising biodegradable interconnected functional groups; and optionally capping groups. Preferably, the sum of interconnected biodegradable functional groups and drug-linker conjugates and capping groups is 16-128, preferred 20-100, more preferred 24-80 and most preferred 30-60.

Preferably, the sum of interconnected functional groups and hydrogel-connected drug-linker conjugates and capping groups of a backbone moiety is equally divided by the number of PEG-based polymeric chains extending from the branching core. For instance, if there are 32 interconnected functional groups and hydrogel-connected drug-linker conjugates and capping groups, eight groups may be provided by each of the four PEG-based polymeric chains extending from the core, preferably by means of dendritic moieties attached to the terminus of each PEG-based polymeric chain. Alternatively, four groups may be provided by each of eight PEG-based polymeric chains extending from the core or two groups by each of sixteen PEG-based polymeric chains. If the number of PEG-based polymeric chains extending from the branching core does not allow for an equal distribution, it is preferred that the deviation from the mean number of the sum of interconnected functional groups and hydrogel-connected drug-linker conjugates and capping groups per PEG-based polymeric chain is kept to a minimum.

In such carrier-linked prodrugs according to the invention, it is desirable that almost all drug release (>90%) has occurred before a significant amount of release of the backbone moieties (<10%) has taken place. This can be achieved by adjusting the carrier-linked prodrug's half-life versus the degradation kinetics of the hydrogel according to the invention.

Preferentially, a backbone moiety is characterized by having a branching core, from which at least three PEG-based polymeric chains extend. Accordingly, in a preferred aspect of the present invention the backbone reagent comprises a branching core, from which at least three PEG-based polymeric chains extend. Such branching cores may be comprised of poly- or oligoalcohols in bound form, preferably pentaerythritol, tripentaerythritol, hexaglycerine, sucrose, sorbitol, fructose, mannitol, glucose, cellulose, amyloses, starches, hydroxyalkyl starches, polyvinylalcohols, dextranes, hyualuronans, or branching cores may be comprised of poly- or oligoamines such as ornithine, diaminobutyric acid, trilysine, tetralysine, pentalysine, hexylysine, heptalysine, octalysine, nonalysine, decalysine, undecalysine, dodecalysine, tridecalysine, tetradecalysine, pentadecalysine or oligolysines, polyethyleneimines, polyvinylamines in bound form.

Preferably, the branching core extends three to sixteen PEG-based polymeric chains, more preferably four to eight. Preferred branching cores may be comprised of pentaerythritol, ornithine, diaminobutyric acid, trilysine, tetralysine, pentalysine, hexylysine, heptalysine or oligolysine, low-molecular weight PEI, hexaglycerine, tripentaerythritol in bound form.

Preferably, the branching core extends three to sixteen PEG-based polymeric chains, more preferably four to eight. Preferably, a PEG-based polymeric chain is a linear poly(ethylene glycol) chain, of which one end is connected to the branching core and the other to a hyperbranched dendritic moiety. It is understood that a polymeric PEG-based chain may be terminated or interrupted by alkyl or aryl groups optionally substituted with heteroatoms and chemical functional groups.

Preferably, a PEG-based polymeric chain is a suitably substituted polyethylene glycol derivative (PEG based).

Preferred structures for corresponding PEG-based polymeric chains extending from a branching core contained in a backbone moiety are multi-arm PEG derivatives as, for instance, detailed in the products list of JenKem Technology, USA (accessed by download from www.jenkemusa.com on Jul. 28, 2009), 4ARM-PEG Derivatives (pentaerythritol core), 8ARM-PEG Derivatives (hexaglycerin core) and 8ARM-PEG Derivatives (tripentaerythritol core). Most preferred are 4arm PEG Amine (pentaerythritol core) and 4arm PEG Carboxyl (pentaerythritol core), 8arm PEG Amine (hexaglycerin core), 8arm PEG Carboxyl (hexaglycerin core), 8arm PEG Amine (tripentaerythritol core) and 8arm PEG Carboxyl (tripentaerythritol core). Preferred molecular weights for such multi-arm PEG-derivatives in a backbone moiety are 1 kDa to 20 kDa, more preferably 1 kDa to 15 kDa and even more preferably 1 kDa to 10 kDa. It is understood that the terminal amine groups of the above mentioned multi-arm molecules are present in bound form in the backbone moiety to provide further interconnected functional groups and reactive functional groups of a backbone moiety.

It is preferred that the sum of interconnected functional groups and reactive functional groups of a backbone moiety is equally divided by the number of PEG-based polymeric chains extending from the branching core. If the number of PEG-based polymeric chains extending from the branching core does not allow for an equal distribution, it is preferred that the deviation from the mean number of the sum of interconnected and reactive functional groups per PEG-based polymeric chain is kept to a minimum.

More preferably, the sum of interconnected and reactive functional groups of a backbone moiety is equally divided by the number of PEG-based polymeric chains extending from the branching core. For instance, if there are 32 interconnected functional groups and reactive functional groups, eight groups may be provided by each of the four PEG-based polymeric chains extending from the core, preferably by means of dendritic moieties attached to the terminus of each PEG-based polymeric chain. Alternatively, four groups may be provided by each of eight PEG-based polymeric chains extending from the core or two groups by each of sixteen PEG-based polymeric chains.

Such additional functional groups may be provided by dendritic moieties. Preferably, each dendritic moiety has a molecular weight in the range of from 0.4 kDa to 4 kDa, more preferably 0.4 kDa to 2 kDa. Preferably, each dendritic moiety has at least 3 branchings and at least 4 reactive functional groups, and at most 63 branchings and 64 reactive functional groups, preferred at least 7 branchings and at least 8 reactive functional groups and at most 31 branchings and 32 reactive functional groups.

Examples for such dendritic moieties are comprised of trilysine, tetralysine, pentalysine, hexylysine, heptalysine, octalysine, nonalysine, decalysine, undecalysine, dodecalysine, tridecalysine, tetradecalysine, pentadecalysine, hexadecalysine, heptadecalysine, octadecalysine, nonadecalysine in bound form. Examples for such preferred dendritic moieties are comprised of trilysine, tetralysine, pentalysine, hexylysine, heptalysine in bound form, most preferred trilysine, pentalysine or heptalysine, ornithine, diaminobutyric acid in bound form.

Most preferably, the hydrogel carrier of the present invention is characterized in that the backbone moiety has a quarternary carbon of formula C(A-Hyp)₄, wherein each A is independently a poly(ethylene glycol) based polymeric chain terminally attached to the quarternary carbon by a permanent covalent bond and the distal end of the PEG-based polymeric chain is covalently bound to a dendritic moiety Hyp, each dendritic moiety Hyp having at least four functional groups representing the interconnected functional groups and reactive functional groups.

Preferably, each A is independently selected from the formula —(CH₂)ₙ₁(OCH₂CH₂)ₙX—, wherein n1 is 1 or 2; n is an integer in the range of from 5 to 50; and X is a chemical functional group covalently linking A and Hyp.

Preferably, A and Hyp are covalently linked by an amide linkage.

Preferably, the dendritic moiety Hyp is a hyperbranched polypeptide. Preferably, the hyperbranched polypeptide comprises lysine in bound form. Preferably, each dendritic moiety Hyp has a molecular weight in the range of from 0.4 kDa to 4 kDa. It is understood that a backbone moiety C(A-Hyp)₄ can consist of the same or different dendritic moieties Hyp and that each Hyp can be chosen independently. Each moiety Hyp consists of between 5 and 32 lysines, preferably of at least 7 lysines, i.e. each moiety Hyp is comprised of between 5 and 32 lysines in bound form, preferably of at least 7 lysines in bound form. Most preferably Hyp is comprised of heptalysinyl.

The reaction of polymerizable functional groups a backbone reagent, more specifically of Hyp with the polymerizable functional groups of polyethyleneglycol based crosslinker reagents results in a permanent amide bond.

Preferably, C(A-Hyp)₄ has a molecular weight in the range of from 1 kDa to 20 kDa, more preferably 1 kDa to 15 kDa and even more preferably 1 kDa to 10 kDa.

One preferred backbone moiety is shown below, dashed lines indicate interconnecting biodegradable linkages to crosslinker moieties and n is an integer of from 5 to 50:

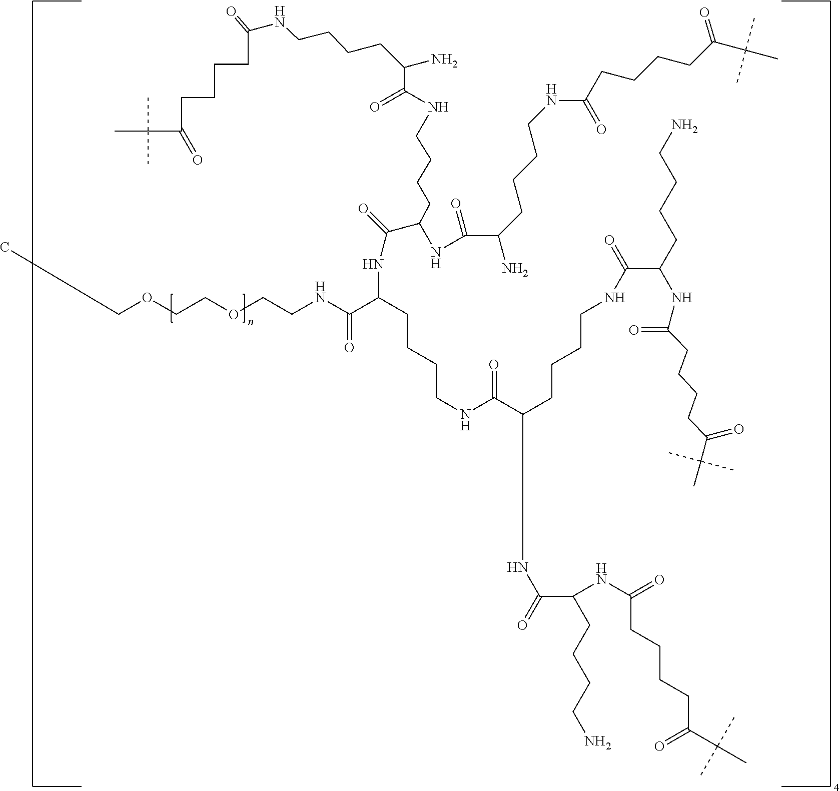

Biodegradability of the hydrogels according to the present invention is achieved by introduction of hydrolytically degradable bonds.

The terms “hydrolytically degradable”, “biodegradable” or “hydrolytically cleavable”, “auto-cleavable”, or “self-cleavage”, “self-cleavable”, “transient” or “temporary” refers within the context of the present invention to bonds and linkages which are non-enzymatically hydrolytically degradable or cleavable under physiological conditions (aqueous buffer at pH 7.4, 37° C.) with half-lives ranging from one hour to three months, including, but are not limited to, aconityls, acetals, amides, carboxylic anhydrides, esters, imines, hydrazones, maleamic acid amides, ortho esters, phosphamides, phosphoesters, phosphosilyl esters, silyl esters, sulfonic esters, aromatic carbamates, combinations thereof, and the like.

If present in a hydrogel according to the invention as degradable interconnected functional group, preferred biodegradable linkages are esters, carbonates, phosphoesters and sulfonic acid esters and most preferred are esters or carbonates.

Permanent linkages are non-enzymatically hydrolytically degradable under physiological conditions (aqueous buffer at pH 7.4, 37° C.) with half-lives of six months or longer, such as, for example, amides.

To introduce the hydrolytically cleavable bonds into the hydrogel carrier of the invention, the backbone moieties can be directly linked to each other by means of biodegradable bonds.

In one embodiment, the backbone moieties of the biodegradable hydrogel carrier may be linked together directly, i.e. without crosslinker moieties. The hyperbranched dendritic moieties of two backbone moieties of such biodegradable hydrogel may either be directly linked through an interconnected functional group that connects the two hyperbranched dendritic moieties. Alternatively, two hyperbranched dendritic moieties of two different backbone moieties may be interconnected through two spacer moieties connected to a backbone moiety and on the other side connected to a crosslinking moiety separated by an interconnected functional groups.

Alternatively, backbone moieties may be linked together through crosslinker moieties, each crosslinker moiety is terminated by at least two of the hydrolytically degradable bonds. In addition to the terminating degradable bonds, the crosslinker moieties may contain further biodegradable bonds. Thus, each end of the crosslinker moiety linked to a backbone moiety comprises a hydrolytically degradable bond, and additional biodegradable bonds may optionally be present in the crosslinker moiety.

Preferably, the biodegradable hydrogel carrier is composed of backbone moieties interconnected by hydrolytically degradable bonds and the backbone moieties are linked together through crosslinker moieties.

The biodegradable hydrogel carrier may contain one or more different types of crosslinker moieties, preferably one. The crosslinker moiety may be a linear or branched molecule and preferably is a linear molecule. In a preferred embodiment of the invention, the crosslinker moiety is connected to backbone moieties by at least two biodegradable bonds.

If present in a hydrogel according to the invention as degradable interconnected functional group, preferred biodegradable linkages are carboxylic esters, carbonates, phosphoesters and sulfonic acid esters and most preferred are carboxylic esters or carbonates.

Preferably, crosslinker moieties have a molecular weight in the range of from 60 Da to 5 kDa, more preferably, from 0.5 kDa to 4 kDa, even more preferably from 1 kDa to 4 kDa, even more preferably from 1 kDa to 3 kDa. In one embodiment, a crosslinker moiety consists of a polymer.

In addition to oligomeric or polymeric crosslinking moieties, low-molecular weight crosslinking moieties may be used, especially when hydrophilic high-molecular weight backbone moieties are used for the formation of a biodegradable hydrogel according to the invention.

Preferably, the poly(ethylene glycol) based crosslinker moieties are hydrocarbon chains comprising ethylene glycol units, optionally comprising further chemical functional groups, wherein the poly(ethylene glycol) based crosslinker moieties comprise at least each methylene glycol units, wherein m is an integer in the range of from 3 to 100, preferably from 10 to 70. Preferably, the poly(ethylene glycol) based crosslinker moieties have a molecular weight in the range of from 0.5 kDa to 5 kDa.

If used in reference to a crosslinker moiety or a PEG-based polymeric chain connected to a branching core, the term “PEG-based” refers to a crosslinker moiety or PEG-based polymeric chain comprising at least 20 weight % ethylene glycol moieties.

In one embodiment, monomers constituting the polymeric crosslinker moieties are connected by biodegradable bonds. Such polymeric crosslinker moieties may contain up to 100 biodegradable bonds or more, depending on the molecular weight of the crosslinker moiety and the molecular weight of the monomer units. Examples for such crosslinker moieties are poly(lactic acid) or poly(glycolic acid) based polymers. It is understood that such poly(lactic acid) or poly(glycolic acid) chain may be terminated or interrupted by alkyl or aryl groups and that they may optionally be substituted with heteroatoms and chemical functional groups.

Preferably, the crosslinker moieties are PEG based, preferably represented by only one PEG based molecular chain. Preferably, the poly(ethylene glycol) based crosslinker moieties are hydrocarbon chains comprising ethylene glycol units, optionally comprising further chemical functional groups, wherein the poly(ethylene glycol) based crosslinker moieties comprise at least each methylene glycol units, wherein m is an integer in the range of from 3 to 100, preferably from 10 to 70. Preferably, the poly(ethylene glycol) based crosslinker moieties have a molecular weight in the range of from 0.5 kDa to 5 kDa.

In a preferred embodiment of the present invention the crosslinker moiety consists of PEG, which is symmetrically connected through ester bonds to two alpha, omega-aliphatic dicarboxylic spacers provided by backbone moieties connected to the hyperbranched dendritic moiety through permanent amide bonds.

The dicarboxylic acids of the spacer moieties connected to a backbone moiety and on the other side is connected to a crosslinking moiety consist of 3 to 12 carbon atoms, most preferably between 5 and 8 carbon atoms and may be substituted at one or more carbon atom. Preferred substituents are alkyl groups, hydroxyl groups or amido groups or substituted amino groups. One or more of the aliphatic dicarboxylic acid's methylene groups may optionally be substituted by O or NH or alkyl-substituted N. Preferred alkyl is linear or branched alkyl with 1 to 6 carbon atoms.

Preferably, there is a permanent amide bond between the hyperbranched dendritic moiety and the spacer moiety connected to a backbone moiety and on the other side is connected to a crosslinking moiety.

One preferred crosslinker moiety is shown below; dashed lines indicate interconnecting biodegradable linkages to backbone moieties:

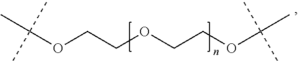

wherein n is an integer of from 5 to 50.

Preferably, the hydrogel carrier is composed of backbone moieties interconnected by hydrolytically degradable bonds.

More preferably, the backbone moieties comprise a branching core of the following formula:

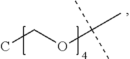

wherein the dashed line indicates attachment to the remainder of the backbone moiety.

More preferably, the backbone moieties comprise a structure of the following formula:

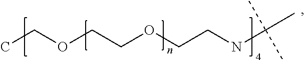

wherein n is an integer of from 5 to 50 and the dashed line indicates attachment to the remainder of the backbone moiety.

Preferably, backbone moiety comprises a hyperbranched moiety Hyp.

More preferably, the backbone moiety comprises a hyperbranched moiety Hyp of the following formula:

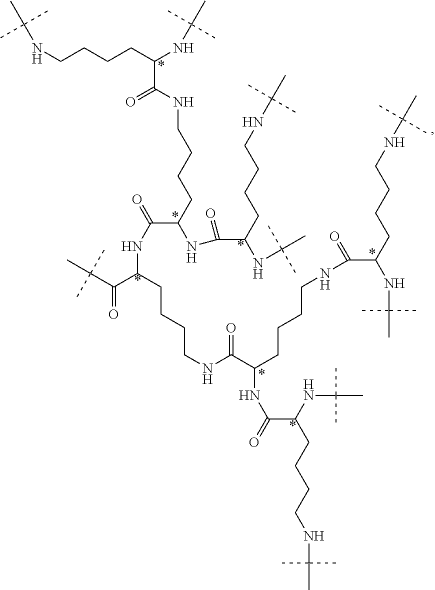

wherein the dashed lines indicate attachment to the rest of the molecule and carbon atoms marked with asterisks indicate S-configuration.

Preferably, the backbone moieties are attached to at least one spacer of the following formula:

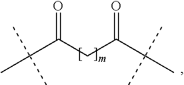

wherein one of the dashed lines indicates attachment to the hyperbranched moiety Hyp and the second dashed line indicates attachment to the rest of the molecule; and
wherein m is an integer of from 2 to 4.

Preferably, the backbone moieties are linked together through crosslinker moieties having the following structure

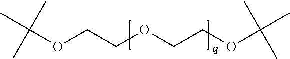

wherein
q is an integer from 3 to 100;
the hydrolysis rate of the biodegradable bonds between backbone moieties and crosslinker moieties is influenced or determined by the number and type of connected atoms adjacent to the PEG-ester carboxy group. For instance, by selecting from succinic, adipic or glutaric acid for PEG ester formation it is possible to vary the degradation half-lives of the biodegradable hydrogel carrier according to the invention.

The degradation of the biodegradable hydrogel carrier according to the invention is a multi-step reaction where a multitude of degradable bonds is cleaved resulting in degradation products which may be water-soluble or water-insoluble. However, water-insoluble degradation products may further comprise degradable bonds so that they can be cleaved in that water-soluble degradation products are obtained. These water-soluble degradation products may comprise one or more backbone moieties. It is understood that released backbone moieties may, for instance, be permanently conjugated to spacer or blocking or linker groups or affinity groups and/or prodrug linker degradation products and that also water-soluble degradation products may comprise degradable bonds.

The structures of the branching core, PEG-based polymeric chains, hyperbranched dendritic moieties and moieties attached to the hyperbranched dendritic moieties can be inferred from the corresponding descriptions provided in the sections covering the hydrogel carriers of the present invention. It is understood that the structure of a degradant depends on the type of hydrogel according to the invention undergoing degradation.

The total amount of backbone moieties can be measured in solution after complete degradation of the hydrogel according to the invention, and during degradation, fractions of soluble backbone degradation products can be separated from the insoluble hydrogel according to the invention and can be quantified without interference from other soluble degradation products released from the hydrogel according to the invention. A hydrogel object according to the invention may be separated from excess water of buffer of physiological osmolality by sedimentation or centrifugation. Centrifugation may be performed in such way that the supernatant provides for at least 10% of the volume of the swollen hydrogel according to the invention. Soluble hydrogel degradation products remain in the aqueous supernatant after such sedimentation or centrifugation step, and water-soluble degradation products comprising one or more backbone moieties are detectable by subjecting aliquots of such supernatant to suitable separation and/or analytical methods.

Preferably, water-soluble degradation products may be separated from water-insoluble degradation products by filtration through 0.45 μm filters, after which the water-soluble degradation products can be found in the flow-through. Water-soluble degradation products may also be separated from water-insoluble degradation products by a combination of a centrifugation and a filtration step.

For instance the backbone moieties may carry groups that exhibit UV absorption at wavelengths where other degradation products do not exhibit UV absorption. Such selectively UV-absorbing groups may be structural components of the backbone moiety such as amide bonds or may be introduced into the backbone by attachment to its reactive functional groups by means of aromatic ring systems such as indoyl groups.

In such hydrogel-linked pramipexole prodrugs according to the invention, it is desirable that almost all pramipexole release (>90%) has occurred before a significant amount of release of the backbone degradation products (<10%) has taken place. This can be achieved by adjusting the hydrogel-linked pramipexole prodrug's half-life versus the hydrogel degradation kinetics.

Optionally, there is a spacer moiety between the carrier and the linker. Preferably, the spacer is connected to the carrier and the linker via stable bonds, such as amide or thiosuccinimide bonds, and preferably these stable bonds are amide bonds. Any spacer known to a person skilled in the art can be used. Preferably, the spacer is a fragment selected from C₁₋₅₀ alkyl, C₂₋₅₀ alkenyl or C₂₋₅₀ alkinyl, which fragment is optionally interrupted by one or more groups selected from —NH—, —N(C₁₋₄ alkyl)-, —O—, —S—, —C(O)—, —C(O)NH—, —C(O)N(C₁₋₄ alkyl)-, —O—C(O)—, —S(O)—, —S(O)₂—, 4 to 7 membered heterocyclyl, phenyl or naphthyl.

In one embodiment of the present invention, the linker L contains a moiety L¹ represented by formula (I),

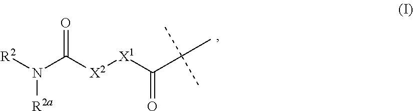

wherein the dashed line indicates the attachment of L¹ to pramipexole by forming an amide bond with the aromatic amino group of pramipexole;
X¹ is C(R¹R^{1a}) or a cyclic fragment selected from C₃₋₇ cycloalkyl, 4 to 7 membered heterocyclyl, phenyl, naphthyl, indenyl, indanyl, tetralinyl, or 9 to 11 membered heterobicyclyl;
X² is a chemical bond or selected from C(R³R^{3a}), N(R³), O, C(R³R^{3a})—C(R⁴R^{4a}), C(R³R^{3a})—N(R⁴), N(R³)—C(R⁴R^{4a}), C(R³R^{3a})—O, or O—C(R³R^{3a}), wherein in case X¹ is a cyclic fragment, X² is a chemical bond, C(R³R^{3a}), N(R³) or O;
optionally, in case X¹ is a cyclic fragment and X² is C(R³R^{3a}), the order of the X¹ fragment and the X² fragment within L¹ may be changed;
R¹, R³ and R⁴ are independently selected from the group consisting of H, C₁₋₄ alkyl and —N(R⁵R^{5a});
R^{1a}, R², R^{2a}, R^{3a}, R^{4a} and a R⁵ are independently selected from the group consisting of H, and C₁₋₄ alkyl;
optionally, one of the pairs R^{2a}/R², R^{2a}/R^{3a}, R^{2a}/R^{4a} are joined to form a 4 to 7 membered at least partially saturated heterocycle;
R⁵ is C(O)R⁶;
R⁶ is C₁₋₄ alkyl;
optionally, one of the pairs R^{1a}/R^{4a}, R^{3a}/R^{4a} or R^{1a}/R^{3a} form a chemical bond;
optionally, L¹ is further substituted.

Preferably, R^{1a}, R², R^{2a}, R^{3a}, R^{4a} and R^{5a} are independently selected from the group consisting of H, and C₁₋₄ alkyl.

More preferably, the moiety L¹ is selected from

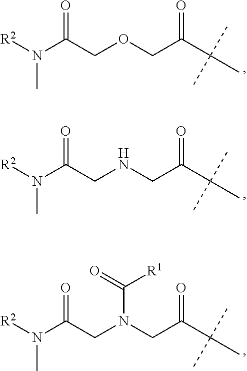

In another preferred embodiment, L is a non-biologically active linker containing a moiety L¹ represented by formula (Ia),

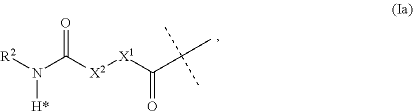

wherein the dashed line indicates the attachment of L¹ to pramipexole by forming an amide bond with the aromatic amino group of pramipexole.
X¹ is C(R¹R^{1a}) or a cyclic fragment selected from C₃₋₇ cycloalkyl, 4 to 7 membered heterocyclyl, phenyl, naphthyl, indenyl, indanyl, tetralinyl, or 9 to 11 membered heterobicyclyl;
X² is a chemical bond or selected from C(R³R^{3a}), N(R³), O, C(R³R^{3a})—C(R⁴R^{4a}), C(R³R^{3a})—N(R⁴), N(R³)—C(R⁴R^{4a}), C(R³R^{3a})—O, or O—C(R³R^{3a}),
wherein in case X¹ is a cyclic fragment, X² is a chemical bond, C(R³R^{3a}), N(R³) or O;
optionally, in case X¹ is a cyclic fragment and X² is C(R³R^{3a}), the order of the X¹ fragment and the X² fragment within L¹ may be changed;
R¹, R³ and R⁴ are independently selected from the group consisting of H, C₁₋₄ alkyl and —N(R⁵R^{5a});
R^{1a}, R², R^{3a}, R^{4a} and R^{5a} are independently selected from the group consisting of H, and C₁₋₄ alkyl;

### R⁵ is C(O)R⁶;

R⁶ is C₁₋₄ alkyl;
optionally, one of the pairs R^{1a}/R^{4a}, R^{3a}/R^{4a} or R^{1a}/R^{3a} form a chemical bond;
optionally, L¹ is further substituted;
provided that the hydrogen marked with the asterisk in formula (I) is not replaced by a substituent or a connection of L¹ to the carrier.

For the sake of clarity it is indicated that the aromatic amino group of pramipexole forms together with the carbonyl-fragment (—C(O)—) on the right hand side of L¹ (as depicted in formula (I)) an amide bond within the carrier linked pramipexole prodrug according to the present invention. By consequence, the two parts of the prodrug are connected (chemically bound) by an amide fragment of the general structure Y¹—C(O)—N(H)—Y². Y¹ indicates the remaining parts of the moiety L¹ and Y² indicates the aromatic fragment of pramipexole. For example, said amide bond is indicated within formula (I)/(Ia) by the dashed line added diagonally on this bond.

Within this embodiment, it is preferred that, in case X¹ is a cyclic fragment, said cyclic fragment is incorporated into L¹ via two adjacent ring atoms and the ring atom of X¹, which is adjacent to the carbon atom of the amide bond, is also a carbon atom; or in case the order of the X¹ fragment and the X² fragment within L¹ is changed, the cyclic fragment is incorporated into L¹ via two adjacent ring atoms.

This means that within the first option of this preferred meaning of said embodiment, the X¹-fragment of the moiety L¹ represented by formula (I)(Ia) may also be a cyclic fragment such as C₃₋₇ cycloalkyl, phenyl or indanyl. In case X¹ is such a cyclic fragment, the respective cyclic fragment is incorporated into L¹ via two adjacent ring atoms (of said cyclic fragment). For example, if X¹ is phenyl, the phenyl fragment of L¹ is bound to the X² fragment of L¹ via a first (phenyl) ring atom being in α-position (adjacent) to a second (phenyl) ring atom, which itself is bound to the carbon atom of the carbonyl-fragment on the right hand side of L¹ according to formula (I)/(Ia) (the carbonyl fragment which forms together with the aromatic amino group of D an amide bond).

It is even more preferred that the moiety L¹ is selected from

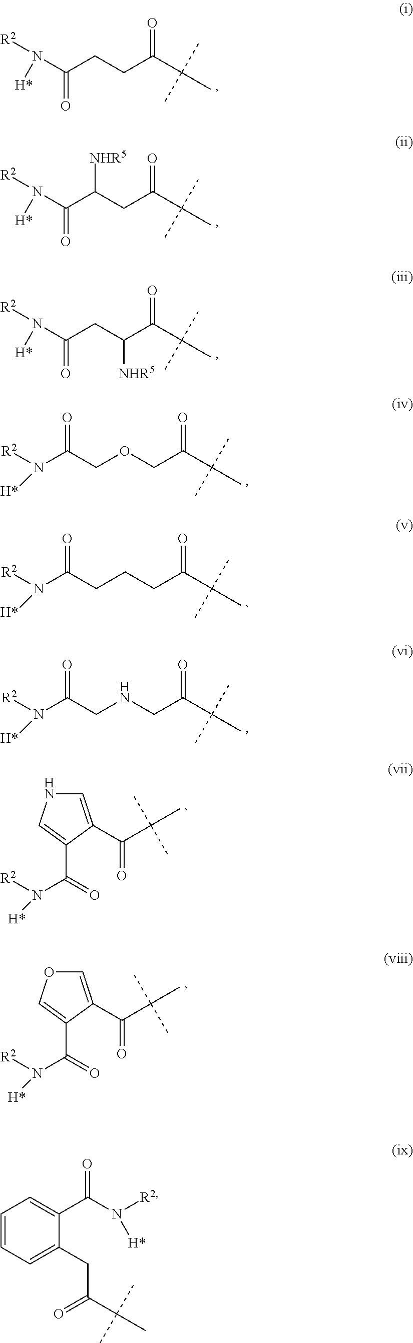

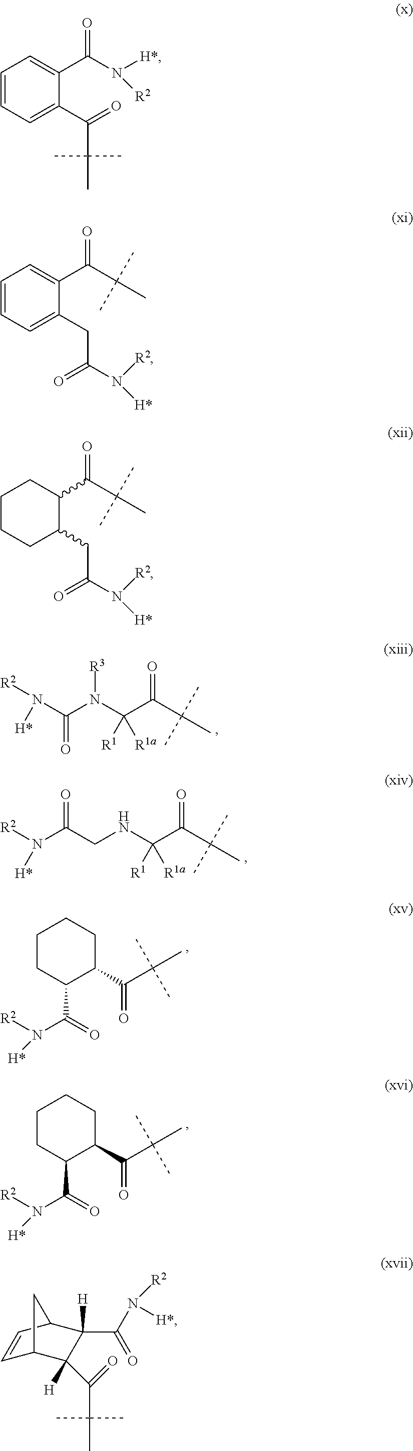

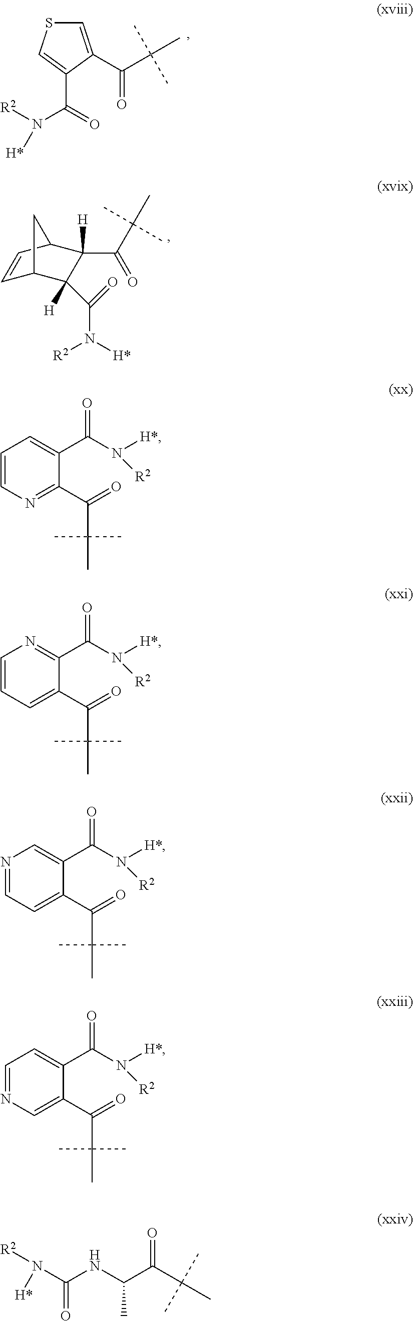

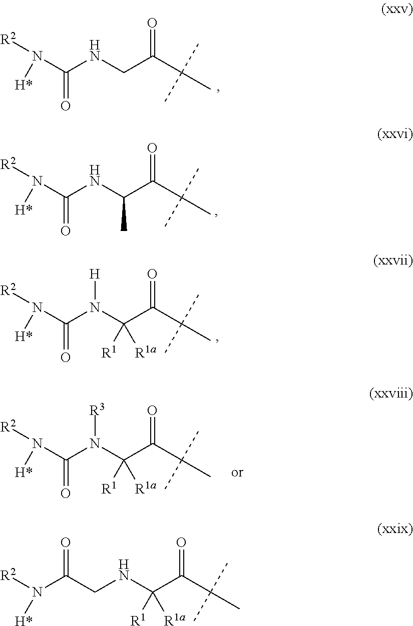

wherein
R⁵ is C(O)R⁶;
R¹, R^{1a}, R², R³ and R⁶ are independently from each other C₁₋₄ alkyl; and
L¹ is optionally substituted with one L² moiety, preferably R² is substituted with one L² moiety.

Preferably, L² is attached to Z through a thiosuccinimide group or amide group, preferably an amide group, which in turn is attached to the hydrogel's backbone moiety through a spacer, such as an oligoethylene glycol chain. Preferably, the linkage of this spacer chain to the backbone moiety is a permanent bond, preferably an amide bond.

Within this embodiment in connection with formulae (I) or (Ia) and the preferred meanings thereof it is also preferred that pramipexole is bound to L, preferably to L¹, via it aromatic amino group.

Within this embodiment of the present invention, in case X¹ is a cyclic fragment and X² is C(R³R^{3a}), the order of X¹ (the X¹ fragment) and X² (the X² fragment) within the moiety L¹ may be changed. This means that in such a case the moiety L¹ is represented by formula (Ib),

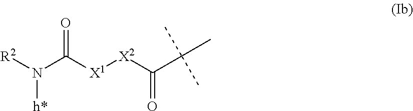

wherein (besides X¹ and X²) all substituents (such as R²) and fragments have the same (chemical) definitions as indicated within the context of the present invention for formula (I).

In a further embodiment of the present invention the linker L contains a moiety L¹ represented by formula (II)

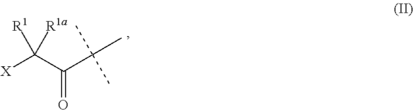

wherein the dashed line indicates the attachment of L¹ to pramipexole by forming an amide bond with the aromatic amino group of pramipexole.
X is H or C₁₋₅₀ alkyl optionally interrupted by one or more groups selected from —NH—, —C(C₁₋₄ alkyl)-, —O—, —C(O)— or —C(O)NH—;
R¹ and R^{1a} are independently selected from the group consisting of H and C₁-C₄ alkyl;
optionally, L¹ is further substituted.

Within this embodiment it is preferred that X is a substituent selected from N(HR²)—C(O)—(CH₂)ₙ—, R³—C(O)—NH—(CH₂)ₙ—, N(HR²)—C(O)—N(H)—, R³—C(O)—NH—, R³—O— or R³—O—(CH₂)ₙ—; and

R² is H or C₁₋₄ alkyl;
R³ is C₁₋₄ alkyl;
n is an integer from 1 to 10.

Preferably, X in formula (II) includes one of the following fragments, wherein the dashed line on the right hand side indicates the attachment of L¹ to pramipexole by forming an amide bond with the aromatic amino group of pramipexole and the dashed line on the left hand side indicates the attachment to the rest of X and wherein L¹ is optionally further substituted:

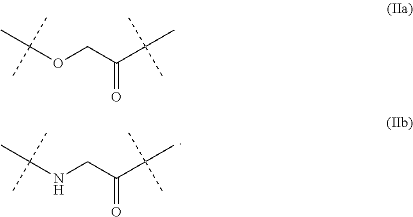

Preferably, X in formula (II) includes one of the following fragments, wherein the dashed line on the right hand side indicates the attachment of L¹ to pramipexole by forming an amide bond with the aromatic amino group of pramipexole and the dashed line on the left hand side indicates the attachment to the rest of X:

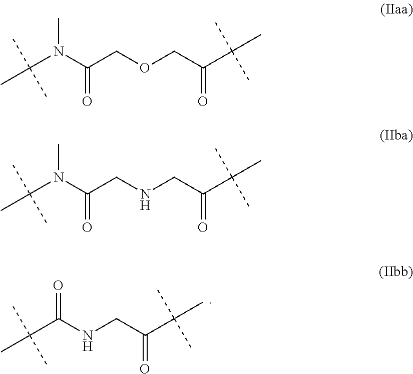

It is even more preferred within this embodiment that pramipexole is bond to L, preferably to L¹, via its aromatic amino group.

The linker L may also contain a moiety L². Preferably, the linker L contains one moiety L¹ as defined above and 1 to 4, preferably 1, L² moieties. In one embodiment of the present invention, the linker L consists of one moiety L¹ and one moiety L².

The moiety L² is a chemical bond or a spacer and L² is bound to the carrier. As indicated above it is preferred that the linker L contains one moiety L¹ represented by, for example, any of the formulas (I)/(Ia)/(Ib) or (II) (including the respective preferred definitions) and 1 to 4 L² moieties, preferably 1 L² moiety. This means that in such a case the moiety L¹ is substituted with the moiety L². On the other hand, the (respective) moiety L² is also bond to the carrier. In other words, the moiety L² connects the carrier with the linker moiety L¹, which in turn is bound to pramipexole.

The moiety L² may be bound to any position of the moiety L¹. However, in case of the definitions of moiety L¹ according to formula (Ia)—including the respective preferred definitions—the hydrogen marked with the asterisk within formula (Ia) is not replaced by the moiety L². The substitution (attachment) of the respective L² moiety occurs by replacing one hydrogen according to the definitions of the moiety L¹ including any fragments or substitutes thereof (for example X¹, X², R¹ to R⁵ and R^{1a} to R^{5a} according to formula I).

In case more than one L² moiety is present (for example 2 to 4 L² moieties), each L² moiety is bond to a carrier as defined above. By consequence, each L² and each carrier can be selected independently.

In case L² is a spacer, any spacer known to a person skilled in the art for connecting a moiety L¹ as represented by, for example, formula (I)/(Ia)/(Ib) to the carrier can be used. Preferably, the spacer is a fragment selected from C₁₋₅₀ alkyl, C₂₋₅₀ alkenyl or C₂₋₅₀ alkinyl, which fragment is optionally interrupted by one or more groups selected from —NH—, —N(C₁₋₄ alkyl)-, —O—, —S—, —C(O)—, —C(O)NH—, —C(O)N(C₁₋₄ alkyl)-, —O—C(O)—, —S(O)—, —S(O)₂—, 4 to 7 membered heterocyclyl, phenyl or naphthyl.

If the moiety L¹ is represented by the embodiment containing formula (I), the spacer L² is more preferably a fragment selected from C₁₋₅₀ alkyl, C₂₋₅₀ alkenyl or C₂₋₅₀ alkinyl, which fragment is optionally interrupted by one or more groups selected from —NH—, —N(C₁₋₄ alkyl)-, —O—, —S—, —C(O)—, —C(O)NH—, —C(O)N(C₁₋₄ alkyl)-, —O—C(O)—, —S(O)—, —S(O)₂—, 4 to 7 membered heterocyclyl, phenyl or naphthyl, provided that the spacer does not contain a nitrogen atom being in β- or γ-position to the amino group containing the hydrogen marked with the asterisk in formula (Ia), in case the spacer is bound to R².

More preferably, the spacer (L²) is a C₁₋₂₀ alkyl being bound to L¹ and which C₁₋₂₀ alkyl is optionally interrupted by one or more groups selected from —NH—, —N(C₁₋₄ alkyl)-, —O—, —S—, —C(O)—, —C(O)NH—, —C(O)N(C₁₋₄ alkyl)-, —O—C(O)—, —S(O)—, —S(O)₂—, 4 to 7 membered heterocyclyl, phenyl or naphthyl. In case of the embodiment containing formula (I), it is also preferred that the spacer does not contain a nitrogen atom being in β- or γ-position to the amino group containing the hydrogen marked with the asterisk in formula (I).

In another embodiment of the present invention it is preferred that in case the moiety L¹ is represented
i) by formula (I), R² is substituted with L²; or
ii) by formula (II), X is substituted with L².

According to another embodiment of the present invention, it is preferred that the moiety L² has a molecular weight in the range of from 14 g/mol to 750 g/mol.

Preferably, L² is attached to the carrier via a terminal group selected from —CO—NH—,

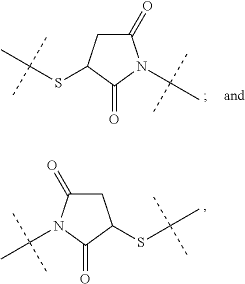

most preferred —CO—NH—.

In case L² has such terminal group it is furthermore preferred that L² has a molecular weight in the range of from 14 g/mol to 500 g/mol calculated without such terminal group.

In another embodiment of the present invention in respect of the definition of L¹ according to formula (I), the spacer (L²) is preferably a C₁₋₂₀ alkyl being bound to R² and which C₁₋₂₀ alkyl is optionally interrupted by one or more groups selected from —NH—, —N(C₁₋₄ alkyl)-, —O—, —S—, —C(O)—, —C(O)NH—, —C(O)N(C₁₋₄ alkyl)-, —O—C(O)—, —S(O)—, —S(O)₂—, 4 to 7 membered heterocyclyl, phenyl or naphthyl, provided that the spacer does not contain a nitrogen atom being in β- or γ-position to the nitrogen atom bound to the hydrogen marked with the asterisk in formula (I) and L² is attached to the carrier via a terminal group selected from —CO—NH—,

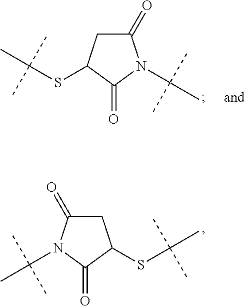

most preferred —CO—NH—,
whereby L² has a molecular weight in the range of from 14 g/mol to 500 g/mol calculated without said terminal group.

In case L² has such terminal group it is furthermore preferred that L² has a molecular weight in the range of from 14 g/mol to 500 g/mol calculated without such terminal group.

Preferably, the covalent attachment formed between the linker and the carrier is a permanent bond and the carrier is a hydrogel.

In one embodiment of the present invention, the linker L of the carrier linked pramipexole prodrug may be optionally substituted further by one or more substituents. The substitution may occur at the moiety L¹ and/or the moiety L² including the respective preferred definitions of L¹ and/or L² as defined above. In general, any substituent may be used as far as the cleavage principle is not affected (when the prodrug is administered to a patient in need thereof.)

Preferably, one or more further optional substituents are independently selected from the group consisting of halogen; CN; COOR⁹; OR⁹; C(O)R⁹; C(O)N(R⁹R^{9a}); S(O)₂N(R⁹R^{9a}); S(O)N(R⁹R^{9a}); S(O)₂R⁹; S(O)R⁹; N(R⁹)S(O)₂N(R^{9a}R^{9b}); SR⁹; N(R⁹R^{9a}); NO₂; OC(O)R⁹; N(R⁹)C(O)R^{9a}; N(R⁹)S(O)₂R^{9a}; N(R⁹)S(O)R^{9a}; N(R⁹)C(O)OR^{9a}; N(R⁹)C(O)N(R^{9a}R^{9b}); OC(O)N(R⁹R^{9a}); T; C₁₋₅₀ alkyl; C₂₋₅₀ alkenyl; or C₂₋₅₀ alkynyl, wherein T; C₁₋₅₀ alkyl; C₂₋₅₀ alkenyl; and C₂₋₅₀ alkynyl are optionally substituted with one or more R¹⁰, which are the same or different and wherein C₁₋₅₀ alkyl; C₂₋₅₀ alkenyl; and C₂₋₅₀ alkynyl are optionally interrupted by one or more groups selected from the group consisting of T, —C(O)O—; —O—; —C(O)—; —C(O)N(R¹¹)—; —S(O)₂N(R¹¹)—; —S(O)N(R¹¹)—; —S(O)₂—; —S(O)—; —N(R¹¹)S(O)₂N(R^{11a})—; —S—; —N(R¹¹)—; —OC(O)R¹¹; —N(R¹¹)C(O)—; —N(R¹¹)S(O)₂—; —N(R¹¹)S(O)—; —N(R¹¹)C(O)O—; —N(R¹¹)C(O)N(R^{11a})—; and —OC(O)N(R¹¹R^{11a});

R⁹, R^{9a}, R^{9b} are independently selected from the group consisting of H; T; and C₁₋₅₀ alkyl; C₂₋₅₀ alkenyl; or C₂₋₅₀ alkynyl, wherein T; C₁₋₅₀ alkyl; C₂₋₅₀ alkenyl; and C₂₋₅₀ alkynyl are optionally substituted with one or more R¹⁰, which are the same or different and wherein C₁₋₅₀ alkyl; C₂₋₅₀ alkenyl; and C₂₋₅₀ alkynyl are optionally interrupted by one or more groups selected from the group consisting of T, —C(O)O—; —O—; —C(O)—; —C(O)N(R¹¹)—; —S(O)₂N(R¹¹)—; —S(O)N(R¹¹)—; —S(O)₂—; —S(O)—; —N(R¹¹)S(O)₂N(R^{11a})—; —S—; —N(R¹¹)—; —OC(O)R¹¹; —N(R¹¹)C(O)—; —N(R¹¹)S(O)₂—; —N(R¹¹)S(O)—; —N(R¹¹)C(O)O—; N(R¹¹)C(O)N(R^{11a})—; and —OC(O)N(R¹¹R^{11a});
T is selected from the group consisting of phenyl; naphthyl; indenyl; indanyl; tetralinyl; C₃₋₁₀ cycloalkyl; 4 to 7 membered heterocyclyl; or 9 to 11 membered heterobicyclyl, wherein T is optionally substituted with one or more R¹⁰, which are the same or different;
R¹⁰ is halogen; CN; oxo (═O); COOR¹²; OR¹²; C(O)R¹²; C(O)N(R¹²R^{12a}); S(O)₂N(R¹²R^{12a}); S(O)N(R¹²R^{12a}); S(O)₂R¹²; S(O)R¹²; N(R¹²)S(O)₂N(R^{12a}R¹²); SR¹²; N(R¹²R^{12a}); NO₂; OC(O)R¹²; N(R¹²)C(O)R^{12a}; MR¹²)S(O)₂R^{12a}; N(R¹²)S(O)R^{12a}; N(R¹²)C(O)OR^{12a}; N(R¹²)C(O)N(R^{12a}R^{12b}); OC(O)N(R¹²R^{12a}); or C₁₋₆ alkyl, wherein C₁₋₆ alkyl is optionally substituted with one or more halogen, which are the same or different;
R¹¹, R^{11a}, R¹², R^{12a}, R^{12b} are independently selected from the group consisting of H; or C₁₋₆ alkyl, wherein C₁₋₆ alkyl is optionally substituted with one or more halogen, which are the same or different.

The carrier linked pramipexole prodrug according to the present invention is any possible combination of pramipexole, the linker L and the carrier as defined above including the respective definitions. Preferred carrier linked pramipexole prodrugs according to the present invention contain a moiety L¹ represented by formula (I),

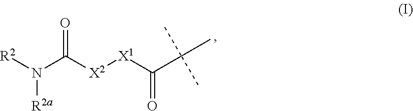

wherein the dashed line indicates the attachment of L¹ to pramipexole by forming an amide bond with the aromatic amino group of pramipexole;
X¹ is C(R¹R^{1a}) or a cyclic fragment selected from C₃₋₇ cycloalkyl, 4 to 7 membered heterocyclyl, phenyl, naphthyl, indenyl, indanyl, tetralinyl, or 9 to 11 membered heterobicyclyl;
X² is a chemical bond or selected from C(R³R^{3a}), N(R³), O, C(R³R^{3a})—C(R⁴R^{4a}), C(R³R^{3a})—N(R⁴), N(R³)—C(R⁴R^{4a}), C(R³R^{3a})—O, or O—C(R³R^{3a}),
wherein in case X¹ is a cyclic fragment, X² is a chemical bond, C(R³R^{3a}), N(R³) or O;
optionally, in case X¹ is a cyclic fragment and X² is C(R³R^{3a}), the order of the X¹ fragment and the X² fragment within L¹ may be changed;
R¹, R³ and R⁴ are independently selected from the group consisting of H, C₁₋₄ alkyl and —N(R⁵R^{5a});
R^{1a}, R², R^{2a}, R^{3a}, R^{4a} and R^{5a} are independently selected from the group consisting of H, and C₁₋₄ alkyl;
optionally, one of the pairs R^{2a}/R², R^{2a}/R^{3a}, R^{2a}/R^{4a} are joined to form a 4 to 7 membered at least partially saturated heterocycle;
R⁵ is C(O)R⁶;
R⁶ is C₁₋₄ alkyl;
optionally, one of the pairs R^{1a}/R^{4a}, R^{3a}/R^{4a} or R^{1a}/R^{3a} form a chemical bond;
optionally, L¹ is further substituted.

In another preferred embodiment, L¹ is represented by formula (Ia),

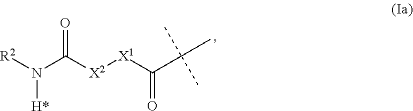

wherein the dashed line indicates the attachment of L¹ to pramipexole by forming an amide bond with the aromatic amino group of pramipexole,
X¹ is C(R¹R^{1a}) or a cyclic fragment selected from C₃₋₇ cycloalkyl, 4 to 7 membered heterocyclyl, phenyl, naphthyl, indenyl, indanyl, tetralinyl, or 9 to 11 membered heterobicyclyl;
X² is a chemical bond or selected from C(R³R^{3a}), N(R³), O, C(R³R^{3a})—C(R⁴R^{4a}), C(R³R^{3a})—N(R⁴), N(R³)—C(R⁴R^{4a}), C(R³R^{3a})—O, or O—C(R³R^{3a}),
wherein in case X¹ is a cyclic fragment, X² is a chemical bond, C(R³R^{3a}), N(R³) or O;
optionally, in case X¹ is a cyclic fragment and X² is C(R³R^{3a}), the order of the X¹ fragment and the X² fragment within L¹ may be changed;
R¹, R³ and R⁴ are independently selected from the group consisting of H, C₁₋₄ alkyl and —N(R⁵R^{5a});
R^{1a}, R², R^{3a}, R^{4a} and R^{5a} are independently selected from the group consisting of H, and C₁₋₄ alkyl;

### R⁵ is C(O)R⁶;

R⁶ is C₁₋₄ alkyl;
optionally, one of the pairs R^{1a}/R^{4a}, R^{3a}/R^{4a} or R^{1a}/R^{3a} form a chemical bond;
optionally, L¹ is further substituted;
provided that the hydrogen marked with the asterisk in formula (I) is not replaced by a substituent or a connection of L¹ to the carrier,
the spacer L² is a fragment selected from C₁₋₅₀ alkyl, C₂₋₅₀ alkenyl or C₂₋₅₀ alkinyl, which fragment is optionally interrupted by one or more groups selected from —NH—, —N(C₁₋₄ alkyl)-, —O—, —S—, —C(O)—, —C(O)NH—, —C(O)N(C₁₋₄ alkyl)-, —O—C(O)—, —S(O)—, —S(O)₂—, 4 to 7 membered heterocyclyl, phenyl or naphthyl, provided that the spacer does not contain a nitrogen atom being in β- or γ-position to the amino group containing the hydrogen marked with the asterisk in formula (I), in case the spacer is bound to R²,
and the carrier is a biodegradable poly(ethylene glycol) (PEG) based water-insoluble hydrogel.

In a preferred embodiment of the present invention, preferred carrier linked pramipexole prodrugs are selected from a prodrug according to the formulas (III) to (VIII),

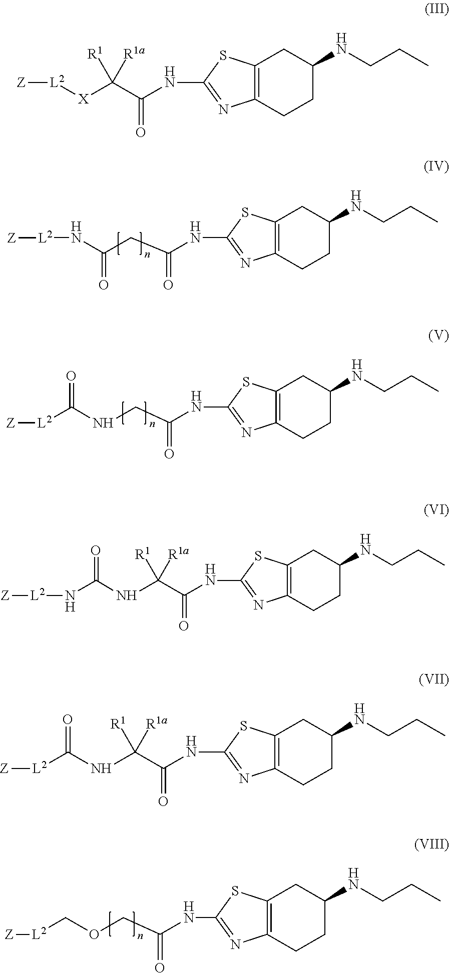

wherein n=1-3, and Z is the carrier;
the carrier and the linker moiety L² are defined as above;
R¹, R^{1a}, X are defined according to formula (II).

In another preferred embodiment of the present invention, preferred carrier linked pramipexole prodrugs are selected from a prodrug according to the formulas (IX) to (XIV),

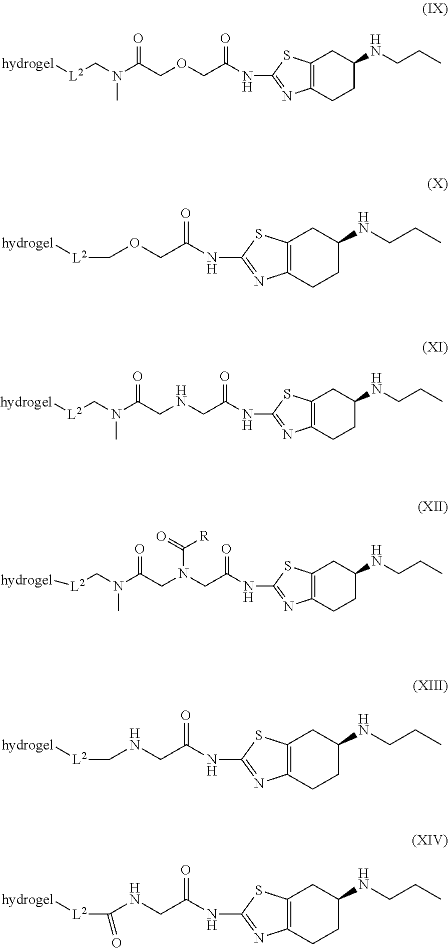

wherein the hydrogel and L² are defined as above;
L² is preferably a spacer.

Another subject of the present invention is a method for the synthesis of a prodrug or a pharmaceutically acceptable salt thereof as defined above. Prodrugs or precursors of prodrugs according to the present invention may be prepared by known methods or in accordance with the reaction sequences described below. The starting materials used in the preparation (synthesis) of prodrugs of the invention or precursors thereof are known or commercially available, or can be prepared by known methods or as described below.

All reactions for the synthesis of the prodrugs according to the present invention including precursors such as the moiety L¹ according to the formulas (I) or (II) are per se well-known to the skilled person and can be carried out under standard conditions according to or analogously to procedures described in the literature, for example in Houben-Weyl, Methoden der Organischen Chemie (Methods of Organic Chemistry), Thieme-Verlag, Stuttgart, or Organic Reactions, John Wiley & Sons, New York. Depending on the circumstances of the individual case, in order to avoid side reactions during the synthesis of a prodrug or a precursor thereof, it can be necessary or advantageous to temporarily block functional groups by introducing protective groups and to deprotect them in a later stage of the synthesis, or introduce functional groups in the form of precursor groups which in a later reaction step are converted into the desired functional groups. Such synthesis strategies and protective groups and precursor groups which are suitable in an individual case are known to the skilled person. If desired, the prodrugs or precursors can be purified by customary purification procedures, for example by recrystallization or chromatography.

The prodrugs according to the present invention (or a pharmaceutically acceptable salt thereof) may be prepared by a method comprising the step of reacting a prodrug precursor L-Y with pramipexole (P) to obtain a pramipexole linker conjugate P-L by forming an amide bond, wherein Y is a leaving group. Afterwards, P-L may be bound to the carrier to obtain the carrier linked prodrugs according to the present invention. Alternatively, the carrier may already be bound to L-Y or L¹-Y (as defined below).

Y is a leaving group. Such leaving groups are known to a person skilled in the art. Preferably, Y is chloride, bromide, fluoride, nitrophenoxy, imidazolyl, N-hydroxysuccinimidyl, N-hydroxybenzotriazolyl, N-hydroxyazobenzotriazolyl, pentafluorophenoxy, 2-thiooxo-thiazolidinyl, or N-hydroxysulfosuccinimidyl.

In case the synthesis of a prodrug according to the present invention is carried out by employing a precursor L¹-Y, a pramipexole linker intermediate (L¹-P) is obtained by reacting L¹-Y with the biologically active drug pramipexole (by forming an amide bond). In such a case, said pramipexole intermediate L¹-P is reacted further to obtain the carrier linked pramipexole product by adding the moiety L² and the carrier to said pramipexole linker intermediate L¹-P. It has to be indicated that the addition of L² and/or the carrier to L¹-P may be performed in several steps by preparing further intermediate compounds prior to obtaining the prodrug according to the present invention.

Alternatively, a prodrug precursor L*-Y may be employed instead of L¹-Y, wherein L* is selected from a fragment of L¹, L¹ containing at least one protecting group or L¹ additionally containing precursors of L² and/or the carrier.

In the following, possible methods of preparing the carrier linked pramipexole prodrugs according to the present invention or intermediates/precursors thereof are explained in more detail.

The hydrogel-linked pramipexole prodrug of the present invention can be prepared starting from the hydrogel of the present invention by convenient methods known in the art. It is clear to a practitioner in the art that several routes exist. For example the prodrug linker mentioned above to which the biologically active moiety is covalently attached can be reacted with the reactive functional groups of the hydrogel of the present invention with or with already bearing the active moiety in part or as whole.

In a preferable method of preparation, the hydrogel is generated through chemical ligation reactions. The hydrogel may be formed from two macromolecular educts with complementary functionalities which undergo a reaction such as a condensation or addition. One of these starting materials is a crosslinker reagent with at least two identical functional groups and the other starting material is a homomultifunctional backbone reagent. Suitable functional groups present on the crosslinker reagent include terminal amino, carboxylic acid and derivatives, maleimide and other alpha,beta unsaturated Michael acceptors like vinylsulfone, thiol, hydroxyl groups. Suitable functional groups present in the backbone reagent include but are not limited to amino, carboxylic acid and derivatives, maleimide and other alpha,beta unsaturated Michael acceptors like vinylsulfone, thiol, hydroxyl groups.

If the crosslinker reagent reactive functional groups are used substoichiometrically with respect to backbone reactive functional groups, the resulting hydrogel will be a reactive hydrogel with free reactive functional groups attached to the backbone structure.

Optionally, the prodrug linker may be first conjugated to pramipexole and the resulting pramipexole-prodrug linker conjugate may then react with the hydrogel's reactive functional groups. Alternatively, after activation of one of the functional groups of the prodrug linker, the linker-hydrogel conjugate may be contacted with pramipexole in the second reaction step and excess pramipexole may be removed by filtration after conjugation of the pramipexole to the hydrogel-bound prodrug linker.

A preferred process for the preparation of a prodrug according to the present invention is as follows:

A preferred starting material for the backbone reagent synthesis is a 4-arm PEG tetra amine or 8-arm PEG octa amine, with the PEG reagent having a molecular weight ranging from 2000 to 10000 Dalton, most preferably from 2000 to 5000 Da. To such multi-arm PEG-derivatives, lysine residues are coupled sequentially to form the hyperbranched backbone reagent. It is understood that the lysines can be partially or fully protected by protective groups during the coupling steps and that also the final backbone reagent may contain protective groups. A preferred building block is bis-boc lysine. Alternatively, instead of sequential additions of lysine residues, a dendritic poly-lysine moiety may be assembled first and subsequently coupled to the 4-arm PEG tetra amine or 8-arm PEG octa amine. It is desirable to obtain backbone reagent carrying 32 amino groups, consequently seven lysines would be attached to each arm of a 4-arm PEG, or five lysines would be attached to each arm of a 8-arm PEG. In another embodiment, the multi-arm PEG derivative is a tetra- or octa carboxy PEG. In this case, the dendritic moieties may be generated from glutaric or aspartic acid, and the resulting backbone reagent would carry 32 carboxy groups. It is understood that all or a fraction of the backbone reagent's functional groups may be present in a free form, as salts or conjugated to protecting groups. It is understood that due to practical reasons the backbone reagent's number of lysines per PEG-arm will be between six and seven, more preferably approximately seven.

A preferred backbone reagent is shown below:

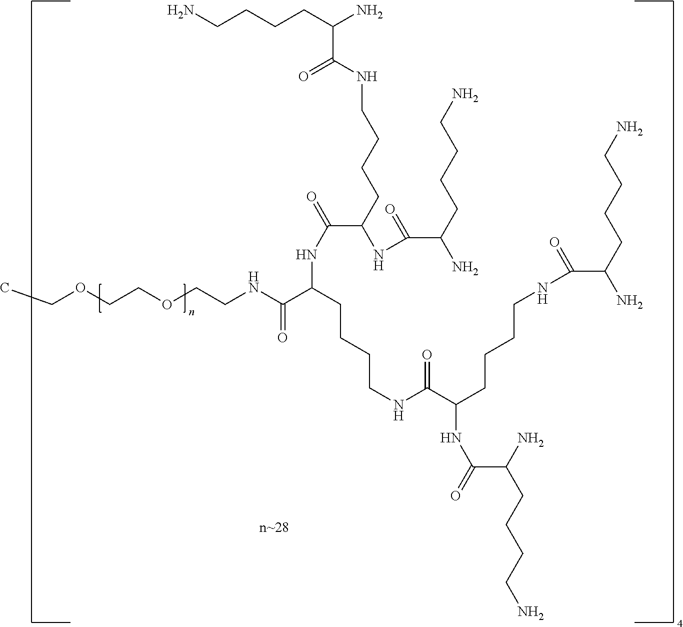

Synthesis of the crosslinker reagent starts from a linear PEG chain with a molecular weight ranging from 0.2 to 5 kDa, more preferably from 0.6 to 2 kDa, which is esterified with a half ester of a dicarboxylic acid, most adipic acid or glutaric acid. Preferred protecting group for half ester formation is the benzylic group. The resulting bis dicarboxylic acid PEG half esters are converted into more reactive carboxy compounds such as acyl chlorides or active esters, eg pentafluorophenyl or N-hydroxysuccinimide esters, most preferred N-hydroxysuccinimde esters, of which preferred selected structure is shown below.

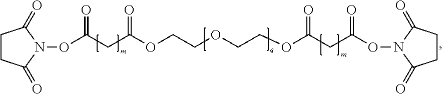

wherein each m independently is an integer ranging from 2 to 4, and
q is an integer of from 3 to 100.

More preferred is the following structure:

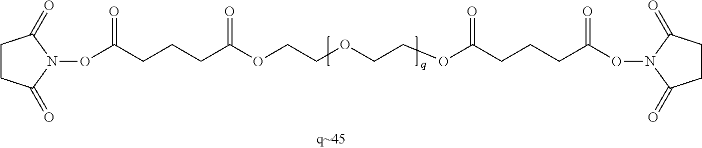

Alternatively, the bis dicarboxylic acid PEG half esters may be activated in the presence of a coupling agent such as DCC or HOBt or PyBOP.

In an alternative embodiment the backbone reagent carries carboxyl groups and the corresponding crosslinker reagent would be selected from ester-containing amino-terminated PEG-chains.

Backbone reagent and crosslinker reagent may be polymerized to form the hydrogel according to the invention using inverse emulsion polymerization. After selecting the desired stoichiometry between backbone and crosslinker polymerizable groups, backbone and crosslinker are dissolved in DMSO and a suitable emulgator with an appropriately selected HLB value, preferably Arlacel P135, is employed to form an inverse emulsion using a mechanical stirrer and controlling the stirring speed. Polymerization is initiated by the addition of a suitable base, preferably by N,N,N′,N′-tetramethylethylenene diamine. After stirring for an appropriate amount of time, the reaction is quenched by the addition of an acid, such as acetic acid and water. The beads are harvested, washed, and fractionated according to particle size by mechanical sieving. Optionally, protecting groups may be removed at this stage.

In an alternative embodiment of this invention, multi-functional moieties are coupled to the reactive functional groups of the polymerized reactive hydrogel to increase the number of functional groups which allows to increase the drug load of the hydrogel. Such multi-functional moieties may be provided by suitably substituted derivatives of lysine, dilysine, trilysine, tetralysine, pentalysine, hexylysine, heptalysine, or oligolysine, low-molecular weight PEI. Preferably, the multi-functional moiety is lysine.

Further, such hydrogel according to the invention may be functionalized with a spacer carrying the same functional group, for instance, amino groups may be introduced into the hydrogel by coupling a heterobifunctional spacer, such as suitably activated COOH-(EG)₆-NH-fmoc (EG=ethylene glycol), and removing the fmoc-protecting group.

In one embodiment, a pramipexole compound may be directly reacted with a reactive biodegradable hydrogel to form a covalent transient linkage resulting in a hydrogel prodrug according to the invention. Such transient linkage between drug and biodegradable hydrogel is preferably a carbamate or amide.

In another embodiment, a pramipexole compound is first conjugated to a spacer in such a fashion that the linkage between drug compound and spacer is a covalent transient linkage such as a carbamate or amide linkage, and is subsequently reacted with the reactive biodegradable hydrogel form a prodrug according to the invention.

In yet another embodiment, a pramipexole compound is first conjugated to a linker in such a fashion that the linkage between drug compound and linker is a covalent transient linkage such as an aromatic amide linkage, and is subsequently reacted with a reactive biodegradable hydrogel to form a prodrug according to the invention.

Further, such hydrogel according to the invention may be functionalized with a spacer carrying a different reactive functional group than provided by the hydrogel. For instance, maleimide reactive functional groups may be introduced into the hydrogel by coupling a suitable heterobifunctional spacer such as Mal-(EG)₆-NHS to the hydrogel. Such functionalized hydrogel can be further conjugated to drug-linker reagents, carrying a reactive thiol group on the linker moiety to form carrier-linked prodrugs according to the present invention.

After loading the pramipexole-linker conjugate to the functionalized maleimido group-containing hydrogel, all remaining functional groups are capped with a suitable blocking reagents, such as mercaptoethanol, to prevent undesired side-reactions.

A particularly preferred method for the preparation of a prodrug of the present invention comprises the steps of

(a) reacting a compound of formula C(A′-X¹)₄, wherein A′-X¹ represents A before its binding to Hyp or a precursor of Hyp and X¹ is a suitable chemical functional group, with a compound of formula Hyp′-X², wherein Hyp′-X² represents Hyp before its binding to A or a precursor of Hyp and X² is a suitable chemical functional group to react with X¹;
(b) optionally reacting the resulting compound from step (a) in one or more further steps to yield a compound of formula C(A-Hyp)₄ having at least four chemical functional groups;
(c) reacting the at least four chemical functional groups of the resulting compound from step (b) with a poly(ethylene glycol) based crosslinker precursor reagent, wherein the crosslinker precursor reagent is used in a sub-stoichiometric amount compared to the total number of functional groups of C(A-Hyp)₄ to yield a hydrogel according to the invention;
(d) reacting remaining un-reacted reactive functional groups (representing the reactive functional groups of the backbone comprised in the reactive biodegradable hydrogel of the present invention) in the hydrogel backbone of step (c) with a covalent conjugate of biologically active moiety and transient prodrug linker or first reacting the un-reacted reactive functional groups with the transient prodrug linker and subsequently with the biologically active moiety;
(e) optionally capping remaining un-reacted reactive functional groups to yield a prodrug of the present invention.

Specifically, hydrogels of the present invention are synthesized as follows:

For bulk polymerization, backbone reagent and crosslinker reagent are mixed in a ratio amine/active ester of 2:1 to 1.05:1.

Both backbone reagent and crosslinker reagent are dissolved in DMSO to give a solution with a concentration of 5 to 50 g per 100 mL, preferably 7.5 to 20 g per 100 ml and most preferably 10 to 20 g per 100 ml.

To effect polymerization, 2 to 10% (vol.) N,N,N′,N′-tertramethylethylene diamine (TMEDA) are added to the DMSO solution containing crosslinker reagent and backbone reagent and the mixture is shaken for 1 to 20 sec and left standing. The mixture solidifies within less than 1 min.

Such hydrogel according to the invention is preferably comminuted by mechanical processes such as stirring, crushing, cutting pressing, or milling, and optionally sieving. For emulsion polymerization, the reaction mixture is comprised of the dispersed phase and the continuous phase.

For the dispersed phase, backbone reagent and crosslinker reagent are mixed in a ratio amine/active ester of 2:1 to 1.05:1 and are dissolved in DMSO to give a to give a solution with a concentration of 5 to 50 g per 100 mL, preferably 7.5 to 20 g per 100 ml and most preferably 10 to 20 g per 100 ml.

The continuous phase is any solvent, that is not miscible with DMSO, not basic, aprotic and shows a viscosity lower than 10 Pa*s. Preferably, the solvent is not miscible with DMSO, not basic, aprotic, shows a viscosity lower than 2 Pa*s and is non-toxic. More preferably, the solvent is a saturated linear or branched hydrocarbon with 5 to 10 carbon atoms. Most preferably, the solvent is n-heptane.

To form an emulsion of the dispersed phase in the continuous phase, an emulsifier is added to the continuous phase before adding the dispersed phase. The amount of emulsifier is 2 to 50 mg per mL dispersed phase, more preferably 5 to 20 mg per mL dispersed phase, most preferably 10 mg per mL dispersed phase.

The emulsifier has an HLB-value of 3 to 8. Preferably, the emulsifier is a triester of sorbitol and a fatty acid or an poly(hydroxyl fatty acid)-poly(ethylene glycol) conjugate. More preferably, the emulsifier is an poly(hydroxy-fatty acid)-polyethylene glycol conjugate, with a linear poly(ethylene glycol) of a molecular weight in the range of from 0.5 kDa to 5 kDa and poly(hydroxy-fatty acid) units of a molecular weight in the range of from 0.5 kDa to 3 kDa on each end of the chain. Most preferably, the emulsifier is poly(ethylene glycol)dipolyhydroxy stearate, Cithrol DPHS (Cithrol DPHS, former Arlacel P135, Croda International Plc).

Droplets of the dispersed phase are generated by stirring with an axial flow impeller with a geometry similar to stirrers such as Isojet, Intermig, Propeller (EKATO Rühr-und Mischtechnik GmbH, Germany)), most preferably similar to Isojet with a diameter of 50 to 90% of the reactor diameter. Preferably, stirring is initiated before addition of the dispersed phase. Stirrer speed is set to 0.6 to 1.7 m/s. The dispersed phase is added at room temperature, and the concentration of the disperse phase is 2% to 70%, preferably 5 to 50%, more preferably 10 to 40%, and most preferably 20 to 35% of the total reaction volume. The mixture of dispersed phase, emulsifier and continuous phase is stirred for 5 to 60 min before adding the base to the effect polymerization.

5 to 10 equivalents (referred to each amide bond to be formed) of a base are added to the mixture of dispersed and continuous phase. The base is aprotic, non nucleophilic and soluble in the disperse phase. Preferably, the base is aprotic, non nucleophilic, well soluble in both disperse phase and DMSO. More preferably, the base is aprotic, non nucleophilic, well soluble in both disperse phase and DMSO, an amine base and non-toxic. Most preferably, the base is N,N,N′,N′-tertramethylethylene diamine (TMEDA). Stirring in the presence of base is continued for 1 to 16 h.

During stirring, droplets of dispersed phase are hardened to become crosslinked hydrogel beads according to the invention which can be collected and fractionation according to size is performed on a vibrational continuous sieving machine with a 75 μm and a 32 μm deck to give hydrogel microparticles according to the invention.

The hydrogel for the prodrug of the present invention can be obtained from the preparation methods in form of micro-particles. In a preferred embodiment of the invention, the reactive hydrogel is a shaped article such as a mesh or a stent. Most preferably, the hydrogel is formed into microparticulate beads which can be administered as subcutaneous or intramuscular injectably by means of a standard syringe. Such soft beads may have a diameter of between 1 and 500 micrometer.

Preferably, such beaded pramipexole hydrogel prodrugs have a diameter of between 10 and 100 micrometer if suspended in an isotonic aqueous formulation buffer, most preferably a diameter of between 20 and 100 micrometer, most preferably a diameter of between 25 and 80 micrometer.

Preferably, such beaded biodegradable hydrogel prodrugs can be administered by injection through a needle smaller than 0.6 mm inner diameter, preferably through a needle smaller than 0.3 mm inner diameter, more preferably through a needle small than 0.25 mm inner diameter, even more preferably through a needle smaller than 0.2 mm inner diameter, and most preferably through a needle small than 0.16 mm inner diameter.

It is understood that the terms “can be administered by injection”, “injectable” or “injectability” refer to a combination of factors such as a certain force applied to a plunger of a syringe containing the biodegradable hydrogel according to the invention swollen in a liquid at a certain concentration (w/v) and at a certain temperature, a needle of a given inner diameter connected to the outlet of such syringe, and the time required to extrude a certain volume of the biodegradable hydrogel carrier according to the invention from the syringe through the needle.

In order to provide for injectability, a volume of 1 mL of the pramipexole prodrugs according to the invention swollen in water to a concentration of at least 5% (w/v) and contained in a syringe holding a plunger of a diameter of 4.7 mm can be extruded at room temperature within 10 seconds by applying a force of less than 50 Newton.

Preferably injectability is achieved for an pramipexole prodrug according to the invention swollen in water to a concentration of ca. 10% (w/v).

Another subject of the present invention is the use of the polymeric pramipexole prodrugs (or a pharmaceutically acceptable salt thereof) as pharmaceuticals or medicaments, respectively. With respect of the use, the same definitions for the polymeric pramipexole prodrug (as well as for further fragments, moieties or substituents such as L¹ or X¹) as laid out above in the context of the prodrug as such apply.

Another subject of the present invention is a pharmaceutical composition comprising an effective dose of at least one prodrug (or a pharmaceutically acceptable salt thereof) as defined above and a pharmaceutically acceptable excipient. Furthermore, the present invention also comprises the use of such pharmaceutical compositions as pharmaceuticals or medicaments, respectively.

Preferably, the pharmaceutically composition is an injectable slow release composition with an effective dose of 10 to 100 mg/mL, based on the quantitative release of free pramipexole, of at least one prodrug or a pharmaceutically acceptable salt thereof.

The effective dose is preferably 15 to 50 mg/ml, most preferably 15 to 35 mg/ml. In addition it is preferred that said pharmaceutical composition releases pramipexole in therapeutical levels for a time period of up to one month, more preferred up to two weeks, most preferred up to one week.

The pharmaceutical composition is further described in the following paragraphs.

The composition of pramipexole hydrogel prodrug may be provided as a suspension composition or as a dry composition. Preferably, the pharmaceutical composition of pramipexole hydrogel prodrug is a dry composition. Suitable methods of drying are, for example, spray-drying and lyophilization (freeze-drying). Preferably, the pharmaceutical composition of pramipexole hydrogel prodrug is dried by lyophilization.

Preferably, the pramipexole hydrogel prodrug is sufficiently dosed in the composition to provide therapeutically effective amount of pramipexole for at least three days in one application. More preferably, one application of the pramipexole hydrogel prodrug is sufficient for one week.

The pharmaceutical composition of pramipexole hydrogel prodrug according to the present invention contains one or more excipients.

Excipients used in parenteral compositions may be categorized as buffering agents, isotonicity modifiers, preservatives, stabilizers, anti-adsorption agents, oxidation protection agents, viscosifiers/viscosity enhancing agents, or other auxiliary agents. In some cases, these ingredients may have dual or triple functions. The compositions of pramipexole hydrogel prodrugs according to the present invention contain one or more than one excipient, selected from the groups consisting of:
(i) Buffering agents: physiologically tolerated buffers to maintain pH in a desired range, such as sodium phosphate, bicarbonate, succinate, histidine, citrate and acetate, sulphate, nitrate, chloride, pyruvate. Antacids such as Mg(OH)₂ or ZnCO₃ may be also used. Buffering capacity may be adjusted to match the conditions most sensitive to pH stability
(ii) Isotonicity modifiers: to minimize pain that can result from cell damage due to osmotic pressure differences at the injection depot. Glycerin and sodium chloride are examples. Effective concentrations can be determined by osmometry using an assumed osmolality of 285-315 mOsmol/kg for serum
(iii) Preservatives and/or antimicrobials: multidose parenteral preparations require the addition of preservatives at a sufficient concentration to minimize risk of patients becoming infected upon injection and corresponding regulatory requirements have been established. Typical preservatives include m-cresol, phenol, methylparaben, ethylparaben, propylparaben, butylparaben, chlorobutanol, benzyl alcohol, phenylmercuric nitrate, thimerosol, sorbic acid, potassium sorbate, benzoic acid, chlorocresol, and benzalkonium chloride
(iv) Stabilizers: Stabilisation is achieved by strengthening of the protein-stabilising forces, by destabilisation of the denatured stater, or by direct binding of excipients to the protein. Stabilizers may be amino acids such as alanine, arginine, aspartic acid, glycine, histidine, lysine, proline, sugars such as glucose, sucrose, trehalose, polyols such as glycerol, mannitol, sorbitol, salts such as potassium phosphate, sodium sulphate, chelating agents such as EDTA, hexaphosphate, ligands such as divalent metal ions (zinc, calcium, etc.), other salts or organic molecules such as phenolic derivatives. In addition, oligomers or polymers such as cyclodextrins, dextran, dendrimers, PEG or PVP or protamine or HSA may be used
(v) Anti-adsorption agents: Mainly ionic or inon-ionic surfactants or other proteins or soluble polymers are used to coat or adsorb competitively to the inner surface of the composition's or composition's container. E.g., poloxamer (Pluronic F-68), PEG dodecyl ether (Brij 35), polysorbate 20 and 80, dextran, poly(ethylene glycol), PEG-poly(histidine), BSA and HSA and gelatines. Chosen concentration and type of excipient depends on the effect to be avoided but typically a monolayer of surfactant is formed at the interface just above the CMC value
(vi) Lyo- and/or cryoprotectants: During freeze- or spray drying, excipients may counteract the destabilising effects caused by hydrogen bond breaking and water removal. For this purpose sugars and polyols may be used but corresponding positive effects have also been observed for surfactants, amino acids, non-aqueous solvents, and other peptides. Trehalose is particularly efficient at reducing moisture-induced aggregation and also improves thermal stability potentially caused by exposure of protein hydrophobic groups to water. Mannitol and sucrose may also be used, either as sole lyo/cryoprotectant or in combination with each other where higher ratios of mannitol:sucrose are known to enhance physical stability of a lyophilized cake. Mannitol may also be combined with trehalose. Trehalose may also be combined with sorbitol or sorbitol used as the sole protectant. Starch or starch derivatives may also be used
(vii) Oxidation protection agents: antioxidants such as ascorbic acid, ectoine, methionine, glutathione, monothioglycerol, morin, poly(ethylene imine) (PEI), propyl gallate, vitamin E, chelating agents such as citric acid, EDTA, hexaphosphate, thioglycolic acid
(viii) Viscosifiers or viscosity enhancers: retard settling of the particles in the vial and syringe and are used in order to facilitate mixing and resuspension of the particles and to make the suspension easier to inject (i.e., low force on the syringe plunger). Suitable viscosifiers or viscosity enhancers are, for example, carbomer viscosifiers like Carbopol 940, Carbopol Ultrez 10, cellulose derivatives like hydroxypropylmethylcellulose (hypromellose, HPMC) or diethylaminoethyl cellulose (DEAE or DEAE-C), colloidal magnesium silicate (Veegum) or sodium silicate, hydroxyapatite gel, tricalcium phosphate gel, xanthans, carrageenans like Satia gum UTC 30, aliphatic poly(hydroxy acids), such as poly(D,L- or L-lactic acid) (PLA) and poly(glycolic acid) (PGA) and their copolymers (PLGA), terpolymers of D,L-lactide, glycolide and caprolactone, poloxamers, hydrophilic poly(oxyethylene) blocks and hydrophobic poly(oxypropylene) blocks to make up a triblock of poly(oxyethylene)-poly(oxypropylene)-poly(oxyethylene) (e.g. Pluronic®), poly(etherester) copolymer, such as a poly(ethylene glycol) terephthalate/poly(butylene terephthalate) copolymer, sucrose acetate isobutyrate (SAIB), dextran or derivatives thereof, combinations of dextrans and PEG, poly(dimethylsiloxane), collagen, chitosan, poly(vinyl alcohol) (PVA) and derivatives, poly(alkylimides), poly(acrylamide-co-diallyldimethyl ammonium (DADMA)), poly(vinylpyrrolidone) (PVP), glycosaminoglycans (GAGs) such as dermatan sulfate, chondroitin sulfate, keratan sulfate, heparin, heparan sulfate, hyaluronan, ABA triblock or AB block copolymers composed of hydrophobic A-blocks, such as polylactide (PLA) or poly(lactide-co-glycolide) (PLGA), and hydrophilic B-blocks, such as poly(ethylene glycol) (PEG) or poly(vinyl pyrrolidone). Such block copolymers as well as the abovementioned poloxamers may exhibit reverse thermal gelation behavior (fluid state at room temperature to facilitate administration and gel state above sol-gel transition temperature at body temperature after injection).
(ix) Spreading or diffusing agent: modifies the permeability of connective tissue through the hydrolysis of components of the extracellular matrix in the intrastitial space such as but not limited to hyaluronic acid, a polysaccharide found in the intercellular space of connective tissue. A spreading agent such as but not limited to hyaluronidase temporarily decreases the viscosity of the extracellular matrix and promotes diffusion of injected drugs.
(x) Other auxiliary agents: such as wetting agents, viscosity modifiers, antibiotics, hyaluronidase. Acids and bases such as hydrochloric acid and sodium hydroxide are auxiliary agents necessary for pH adjustment during manufacture

Preferably, the composition of pramipexole hydrogel prodrug contains one or more than one viscosifier and/or viscosity modifying agent.

The term “excipient” preferably refers to a diluent, adjuvant, or vehicle with which the therapeutic is administered. Such pharmaceutical excipient can be sterile liquids, such as water and oils, including those of petroleum, animal, vegetable or synthetic origin, including but not limited to peanut oil, soybean oil, mineral oil, sesame oil and the like. Water is a preferred excipient when the pharmaceutical composition is administered orally. Saline and aqueous dextrose are preferred excipients when the pharmaceutical composition is administered intravenously. Saline solutions and aqueous dextrose and glycerol solutions are preferably employed as liquid excipients for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsions, tablets, pills, capsules, powders, sustained-release formulations and the like. The composition can be formulated as a suppository, with traditional binders and excipients such as triglycerides. Oral formulation can include standard excipients such as pharmaceutical grades of mannitol, lactose, starch, magnesium stearate, sodium saccharine, cellulose, magnesium carbonate, etc. Examples of suitable pharmaceutical excipients are described in “Remington's Pharmaceutical Sciences” by E. W. Martin. Such compositions will contain a therapeutically effective amount of the therapeutic, preferably in purified form, together with a suitable amount of excipient so as to provide the form for proper administration to the patient. The formulation should suit the mode of administration.

In one embodiment of the present invention, the dry composition of pramipexole hydrogel prodrug is provided as a single dose, meaning that the container in which it is supplied contains one pharmaceutical dose.

In another aspect of the present invention the composition is provided as a single dose composition.

In a general embodiment a pharmaceutical composition of the present invention whether in dry form or as a suspension or in another form may be provided as single or multiple dose composition.

Preferably, the suspension composition or dried composition is a multiple dose composition, meaning that it contains more than one therapeutic dose. Preferably, a multiple dose composition contains at least 2 doses. Such multiple dose composition of pramipexole hydrogel can either be used for different patients in need thereof or is intended for use in one patient, wherein the remaining doses are stored after the application of the first dose until needed.

In another aspect of the present invention the composition is comprised in a container. Preferably the container is a dual-chamber syringe. Especially the dry composition according to the present invention is provided in a first chamber of the dual-chamber syringe and reconstitution solution is provided in a second chamber of the dual-chamber syringe.

Prior to applying the dry composition of pramipexole hydrogel prodrug to a patient in need thereof, the dry composition is reconstituted. Reconstitution can take place in the container in which the dry composition of pramipexole hydrogel prodrug is provided, such as in a vial, syringe, dual-chamber syringe, ampoule, and cartridge. Reconstitution is done by adding a predefined amount of reconstitution solution to the dry composition. Reconstitution solutions are sterile liquids, such as water or buffer, which may contain further additives, such as preservatives and/or antimicrobials, such as, for example, benzylalcohol and cresol. Preferably, the reconstitution solution is sterile water.

An additional aspect of the present invention relates to the method of administration of a reconstituted pramipexole hydrogel prodrug composition. The pramipexole hydrogel prodrug composition can be administered by methods of injection or infusion, including intradermal, subcutaneous, intramuscular, intravenous, intraosseous, and intraperitoneal. Preferably, the pramipexole hydrogel prodrug is administered subcutaneously.

A further aspect is a method of preparing a reconstituted composition comprising a therapeutically effective amount of an pramipexole hydrogel prodrug, and optionally one or more pharmaceutically acceptable excipients, wherein the pramipexole is transiently linked to a hydrogel, the method comprising the step of
contacting the composition of the present invention with a reconstitution solution.

Another aspect is a reconstituted composition comprising a therapeutically effective amount of a pramipexole hydrogel prodrug, and optionally one or more pharmaceutically acceptable excipients, wherein the pramipexole is transiently linked to a hydrogel obtainable by the method above.

Another aspect of the present invention is the method of manufacturing a dry composition of pramipexole hydrogel prodrug. In one embodiment, such suspension composition is made by
(i) admixing the pramipexole hydrogel prodrug with one or more excipients,
(ii) transferring amounts equivalent to single or multiple doses into a suitable container,
(iii) drying the composition in said container, and
(iv) sealing the container.

Suitable containers are vials, syringes, dual-chamber syringes, ampoules, and cartridges.

Another aspect is a kit of parts. When the administration device is simply a hypodermic syringe then the kit may comprise the syringe, a needle and a container comprising the dry pramipexole hydrogel prodrug composition for use with the syringe and a second container comprising the reconstitution solution. In more preferred embodiments, the injection device is other than a simple hypodermic syringe and so the separate container with reconstituted pramipexole hydrogel prodrug is adapted to engage with the injection device such that in use the liquid composition in the container is in fluid connection with the outlet of the injection device. Examples of administration devices include but are not limited to hypodermic syringes and pen injector devices. Particularly preferred injection devices are the pen injectors in which case the container is a cartridge, preferably a disposable cartridge.

A preferred kit of parts comprises a needle and a container containing the composition according to the present invention and optionally further containing a reconstitution solution, the container being adapted for use with the needle. Preferably, the container is a dual-chamber syringe.

In another aspect, the invention provides a cartridge containing a composition of pramipexole hydrogel prodrug as hereinbefore described for use with a pen injector device. The cartridge may contain a single dose or multiplicity of doses of pramipexole.

Examples of diseases, which can be treated by employing the prodrugs and/or the pharmaceutical compositions according to the present invention are dopamine receptor related diseases, including Parkinson's disease, neurological disorders, amyotrophic lateral sclerosis, compulsive behavior, bipolar disorders, Tourette's syndrome, depressive disorders, treatment resistant depression, fibromyalia or restless leg syndrome (RLS).

Preferred diseases to be treated are Parkinson's disease or RLS.

The use of the prodrugs and/or the pharmaceutical compositions according to the present invention includes the prophylaxis and/or treatment of said diseases. The present invention also includes a method for producing a medicament for the prophylaxis and/or treatment of said diseases. The present invention also includes a method of treating, controlling, delaying or preventing in a mammalian patient in need of the treatment of one or more conditions comprising administering to said patient a therapeutically effective amount of a prodrug (or a pharmaceutically acceptable salt thereof) according to the present invention or a respective pharmaceutical composition.

All prodrugs according to the present invention or the respective pharmaceutical compositions can be administered to animals, preferably to mammals, and in particular to humans. The prodrugs and/or pharmaceutical compositions can be administered as such or in mixtures with one another or in mixtures with other pharmaceuticals. The prodrugs and/or the respective pharmaceutical compositions according to the present invention are administered in effective doses, which are known to a person skilled in the art.

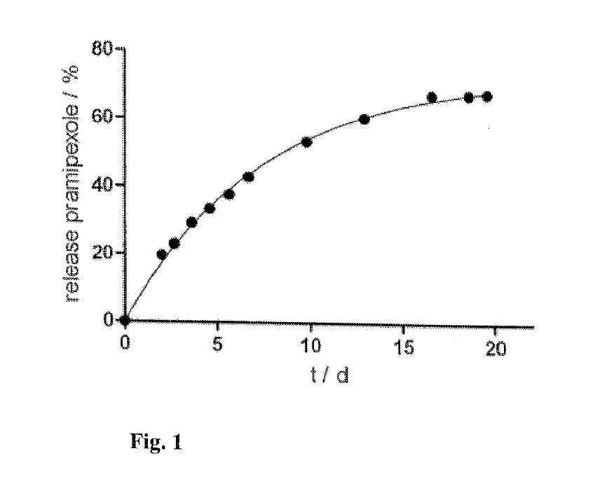

FIG. 1 additionally depicts the in vitro release kinetic of the carrier linked pramipexole prodrug of example 16a. The x-axis shows the time [unit: days].

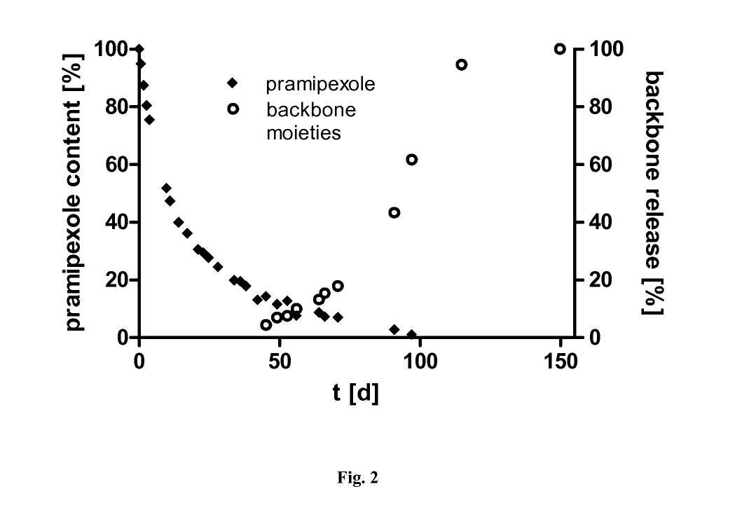

FIG. 2 shows the release kinetics and hydrogel degradation kinetics of the pramipexole linker hydrogel 30.

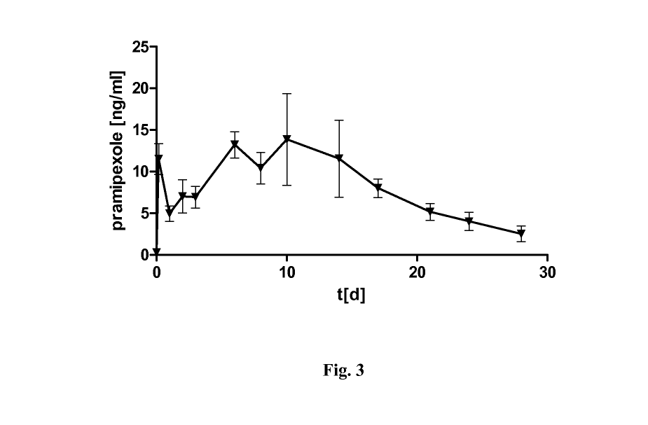

FIG. 3 shows a single dose pharmacokinetics of the pramipexole linker hydrogel 30.

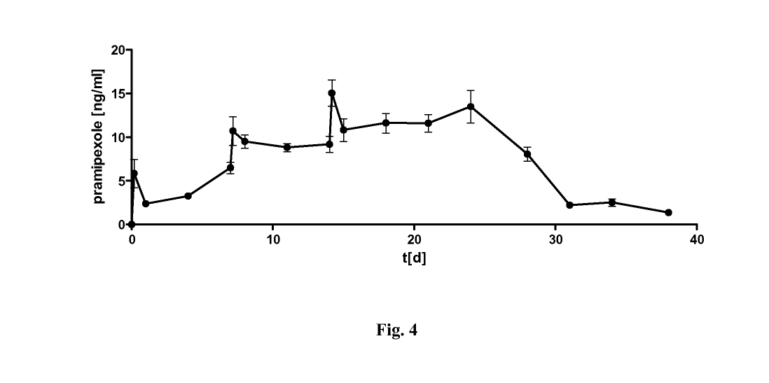

FIG. 4 shows a sustained release of 26 with low peak to trough ratios was observed after three repeated subcutaneous injections.

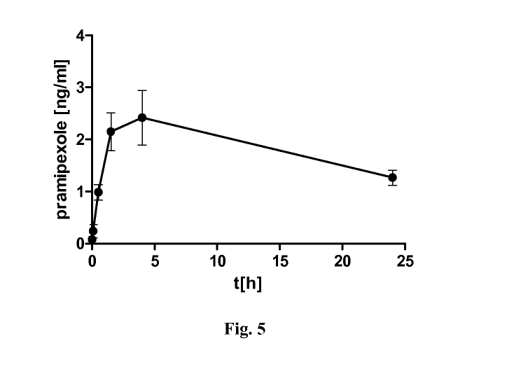

FIG. 5 shows a burstless release of pramipexole over 24 hours was observed after a single dose subcutaneous injection.

The following examples illustrate the invention without limitation.

### Materials and Methods

2-Chlorotrityl chloride resin and Sieber amide resin were obtained from Merck Biosciences GmbH, Schwalbach/Ts, Germany. Boc-Gly-OH and Fmoc-Gly-OH were obtained from Merck KGaA, Darmstadt, Germany. Ac-Glu(OtBu)-OH was obtained from Bachem, Bubendorf, Switzerland. Mal-dPEG6-NHS-ester was obtained from celares GmbH, Berlin, Germany. Amino 4-arm PEG5000 was obtained from JenKem Technology, Beijing, P. R. China. Amino 4-arm PEG2000 was obtained from CreativePEGWorks, Winston Salem, N.C., USA. Pramipexole dihydrochloride was obtained from Carbone Scientific Co., Ltd., Wuhan, China. Fmoc-PP-OH was obtained from Polypure AS, Oslo, Norway. Fmoc-Ado-OH was obtained from PolyPeptide, Strasbourg, France. All other chemicals were obtained from Sigma-ALDRICH Chemie GmbH, Taufkirchen, Germany.

S-Tritylcysteamine was synthesized according to the literature: Di Maro, S. Pong, R.- C. Hsieh, J.- T. Ahn, J.- M. J. Med. Chem. 2008, 51(21), 6639-6641.

6-Tritylsulfanylhexane-1-amine was synthesized according to the literature: Raghunand, N. Jagadish, B. Trouard, T. P.; Galons, J.- P.; Gillies, R. J.; Mash, E. A. Magnetic Resonance in Medicine 2006, 55(6), 1272-1280.

Solid phase synthesis was performed on 2-Chlorotrityl chloride resin with a loading of 1.1-1.0 mmol/g or Sieber amide resin with a loading of 0.64-0.62 mmol/g. Syringes equipped with polyethylene frits were used as reaction vessels.

Loading of the first amino acid to resins was performed according to manufacturer's instructions.

### Fmoc Deprotection:

For Fmoc protecting-group removal, the resin was agitated with 2/2/96 (v/v/v) piperidine/DBU/DMF (two times, 10 min each) at RT and washed with DMF (ten times).

### Standard Solid Phase Coupling Conditions for Acids:

Coupling of acids (aliphatic acids, Fmoc-amino acids) to free amino groups on resin was achieved by agitating resin with 2 eq of acid, 2 eq PyBOP and 4 eq DIEA in relation to free amino groups on resin (calculated based on theoretical loading of the resin) in DMF at RT. After 1 hour resin was washed with DMF (10 times).

### 3-Maleimido Propionic Acid Coupling:

Coupling of 3-maleimidopropionic acid to free amino groups on resin was achieved by agitating resin with 2 eq of acid, 2 eq DIC and 2 eq HOBt in relation to free amino groups in DMF at RT. After 30 min, the resin was washed with DMF (10 times).

### Standard Protocol for the Synthesis of Ureas on Resin:

Synthesis of ureas starting from free amino groups on resin was achieved by agitating resin with 2.5 eq of bis(pentafluorophenyl)carbonate and 5 eq DIEA in relation to free amino groups in DCM at RT. After 45 min resin was washed with DMF (10 times). 1 eq of amine and 2.5 eq DIEA were dissolved in DCM. Mixture was added to resin and agitated for 75 min at RT. Resin was washed with DMF (10 times).

### Cleavage Protocol for Sieber Amide Resin:

Upon completed synthesis, the resin was washed with DCM (10 times), dried in vacuo and treated with 96/2/2 (v/v) DCM/TES/TFA (three times, 15 min each). Eluates were combined, volatiles were removed under a nitrogen stream and product was purified by RP-HPLC.

### Cleavage Protocol for 2-Chlorotrityl Chloride Resin:

Upon completed synthesis, the resin was washed with DCM, dried in vacuo and treated three times for 30 minutes with 7/3 (v/v) DCM/HFIP. Eluates were combined, volatiles were removed under reduced pressure and product was purified by RP-HPLC.

### RP-HPLC Purification:

RP-HPLC was done on a 100×20 or a 100×40 mm C18 ReproSil-Pur 3000DS-3 5μ column (Dr. Maisch, Ammerbuch, Germany) connected to a Waters 600 HPLC System and Waters 2487 Absorbance detector. Linear gradients of solution A (0.1% TFA in H₂O) and solution B (0.1% TFA in acetonitrile) were used. HPLC fractions containing product were pooled and lyophilized.

### HPLC-MS Analytics:

Ultra performance liquid chromatography-electronspray ionization mass spectrometry (HPLC-ESI-MS) was performed on a Waters Acquity Ultra Performance LC instrument connected to a Thermo scientific LTQ Orbitrap Discovery instrument and spectra were, if necessary, interpreted by Thermo scientific software xcalibur.

In general the m/z signal corresponding to the most abundant isotope is given.

Mass spectra of polydisperse PEG products showed a series of (CH₂CH₂O)ₙ moieties due to polydispersity of PEG starting materials. For easier interpretation only one single representative m/z signal is given in the examples.

### Hydrogel Free Amino Group Quantifiction:

The amount of amino groups was determined according to a method used for amino group quantification of solid phase synthesis resins (M. Gude, J. Ryf, P. D. White, Lett. Pept. Sci., 2002, 9, 203-206).

### Quantification of Pramipexole Content in Hydrogel Linker Pramipexole:

A 50 μl aliquot of Hydrogel-linker pramipexole in acetate pH 5 buffer was hydrolyzed by mixing with 1 ml of 1 M HCl and incubating for 72 h at 37° C. Hydrolysate was diluted 1:10 with 0.5 M phosphate buffer pH 7.4 and assayed for pramipexole content by means of SEC (Aekta Explorer system equipped with a Superdex75 5/150 GL column, GE Healthcare, eluent: 20 mM phosphate buffer pH 7.4, 150 mM NaCl, flow: 0.35 ml/min). Pramipexole signal at 3.35 ml was integrated (263 nm) and pramipexole content was calculated according to a pramipexole calibration curve.

### Quantification of Pramipexole in In Vitro Release Studies:

In vitro release sample was vortexed and centrifuged. An aliquot was taken from the supernatant, diluted with eluent (20 mM phosphate buffer pH 7.4, 150 mM NaCl) and assayed for pramipexole content by SEC as described above.

### Analysis of Hydrogel Degradation:

Hydrogel degradation was analysed by monitoring release of water soluble (backbone moieties containing) macromonomers from hydrogel by SEC. Sample preparation and SEC conditions are identical as given in “Quantification of pramipexole in in vitro release studies”. Signals of macromonomers at 0.9-1.7 min were integrated (215 nm) and plotted versus time.

### Quantification of Pramipexole in Rat Plasma:

Quantification of pramipexole in rat plasma was performed by Liquid Chromatography Tandem Mass Spectrometry using memantine as internal standard.

Quantifications were carried out using a Waters Acquity HPLC coupled to a Thermo LTQ Orbitrap Discovery mass spectrometer via an ESI probe and with Waters Amide (50×2.1 mm I.D., 1.7 μm particle size) as analytical column (mobile phase A: 10 mM ammonium bicarbonate pH 8.2, mobile phase B: acetonitrile, T=30° C.). The gradient system comprised a linear gradient from 0.1% B to 99% B in 4 min, an isocratic washing phase with 99% B (1.0 min), and a reconditioning phase (2.9 min) with a flow rate of 0.25 mL/min. Detection of the ions was performed in the selected reaction monitoring (SRM) mode, monitoring the transition pairs at the m/z 212.1 precursor ion to the m/z 153.0 product ion for pramipexole and m/z 180.2 precursor ion to the m/z 164.2 product ion for the internal standard memantine.

Frozen plasma samples (˜95 μL) were thawed on ice for 30 min before analysis. NaOH (50 μL, 0.5 M NaOH) was added and samples were spiked with 88 pg memantine HCl (20 μl of an aqueous memantine HCl solution c=4.4 pg/μL) and extracted twice with diethyl ether (2×500 μL). Technically the aqueous layer was frozen in a liquid nitrogen bath and the organic layer was transferred into a separate tube. Analyte and internal standard were re-extracted from the combined organic layers with 100 μL 0.1 N HCl. Samples were centrifugated and the aqueous layer was frozen in a liquid nitrogen bath. The organic phases were withdrawn and the remaining aqueous layers were lyophilized. Lyophilisates were dissolved in 10 mM ammonium formate pH 4.6 (60 μL) and 2 aliquots (2×15 μA) were injected into the HPLC-MS system, the first aliquot for pramipexole analysis, and the second for internal standard analysis.

The calibration curve was acquired by plotting the ratio (peak area pramipexole):(peak area internal standard) against the nominal amount of calibration standards. The results were fitted to linear regression using standard software.

The ratios (peak area pramipexole):(peak area internal standard) of the quantification experiments at different time points and the calibration curve were used to calculate the pramipexole concentration in rat plasma (ng mL⁻¹).

### EXAMPLE 1

### Synthesis of Linker Pramipexole Conjugate (1b)

### Synthesis of Intermediate (1a):

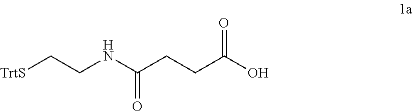

S-Tritylcysteamine (100 mg, 0.313 mmol), succinic anhydride (323 mg, 3.130 mmol) and DIEA (273 μL, 1.567 mmol) were dissolved in dry DCM (2.2 mL) and agitated for 30 min at RT. The mixture was acidified by addition of AcOH (0.7 mL), diluted with diethyl ether and washed twice with water. The organic phase was dried over MgSO₄, the solvent was evaporated under reduced pressure.

Yield: 95 mg (0.226 mmol).

MS: m/z 442.1=[M+Na]⁺ (MW calculated=419.6 g/mol).

### Synthesis of (1b):

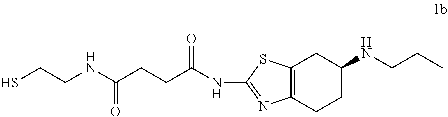

Acid 1a (30 mg, 0.072 mmol), PyBOP (45 mg, 0.086 mmol) and N-methyl morpholine (79 μL, 0.715 mmol) were dissolved in DMSO (1 mL). Pramipexole dihydrochloride (81 mg, 0.286 mmol) was added and the mixture was stirred for 16 h. The reaction was quenched by addition of acetic acid and the mixture was diluted with 3.5 mL acetonirile/water 1/1+0.1% TFA. The trityl protected intermediate of 1b was purified by RP-HPLC. After lyophilisation 21 mg (0.029 mmol) of the TFA salt were obtained.

MS: m/z 613.4=[M+H]⁺ (MW calculated=612.9 g/mol).

For trityl deprotection the lyophilisate was dissolved in HFIP (2 mL), TES (20 μL) was added, and the mixture was incubated for 10 min. Volatiles were evaporated and 1b was purified by RP-HPLC.

Yield: 12 mg (0.025 mmol, TFA salt).

MS: m/z 371.2=[M+H]⁺ (MW calculated=370.5).

### EXAMPLE 2

### Synthesis of Linker Pramipexole Conjugate (2b)

### Synthesis of Intermediate (2a)

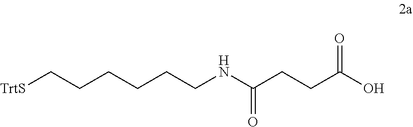

2a was synthesized as described for 1a except for the use of 6-tritylsulfanylhexane-1-amine instead of S-tritylcysteamine.

Yield: 170 mg (0.226 mmol).

MS: m/z 498.2=[M+Na]⁺ (MW calculated=475.7 g/mol).

### Synthesis of Linker Pramipexole Conjugate (2b)

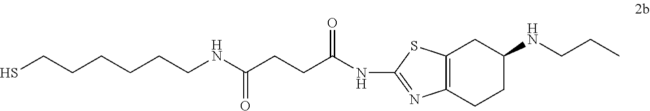

2b was synthesized as described for 1b except for the use of 2a instead of 1a.

2b: Yield: 3.5 mg (0.006 mmol, TFA salt).

MS: m/z 427.2=[M+H]⁺ (MW calculated=426.7 g/mol).

### EXAMPLE 3

### Synthesis of Linker Pramipexole Conjugate (3b)

### Synthesis of Intermediate (3a)

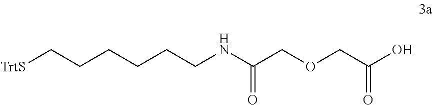

3a was synthesized as described for 2a except for the use of 1,4-dioxane-2,6-dione instead of succinic anhydride.

Yield: 117 mg (0.237 mmol).

MS: m/z 983.4=[2M+H]⁺ (MW calculated=491.7 g/mol).

### Synthesis of Linker Pramipexole Intermediate (3b)

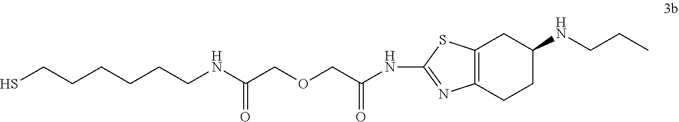

3b was synthesized as described for 1b except for the use of 3a instead of 1a. The coupling of pramipexole was completed within 30 min.

3b: Yield: 4.5 mg (0.008 mmol, TFA salt).

MS: m/z 443.2=[M+H]⁺ (MW calculated=442.7 g/mol).

### EXAMPLE 4

### Synthesis of Linker Pramipexol Conjugate (4b)

### Synthesis of Intermediate (4a):

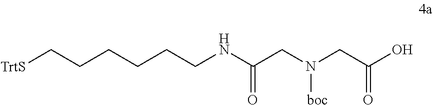

4a was synthesized as described for 2a except for the use of tert-butyl 2,6-dioxomorpholine-4-carboxylate instead of succinic anhydride.

Yield: 148 mg (0.250 mmol).

MS: m/z 591.3=[M+H]⁺ (MW calculated=590.8 g/mol).

### Synthesis of Linker Pramipexole Conjugate (4b)

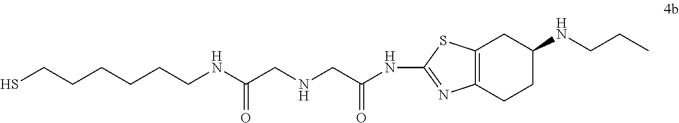

4b was synthesized as described for 1b except for the use of 4a instead of 1a. The coupling of pramipexole was completed within 40 min. 38 mg (0.042 mmol, TFA salt) of the coupling product were isolated after RP-HPLC purification and lyophilisation.

MS: m/z 784.4=[M+H]⁺ (MW calculated=784.1 g/mol).

11 mg of the intermediate were used for deprotection of the thiol. For protecting group removal the intermediate was dissolved in 1.2 mL HFIP/TFA (1/1), 48 μL of TES/water (1/1) was added, and the solution was agitated for 1.5 h. Volatiles were removed and the product was purified by RP-HPLC.

Yield: 7.7 mg (0.011 mmol, double TFA salt).

MS: m/z 442.2=[M+H]⁺ (MW calculated=441.7 g/mol).

### EXAMPLE 5

### Synthesis of Linker Pramipexole Conjugate (5c)

### Synthesis of Intermediate (5a)

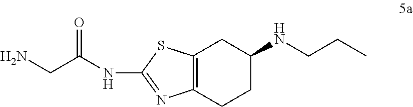

Boc-Gly-OH (659 mg, 3.76 mmol), PyBOP (2.35 g, 4.51 mmol) and N-methyl morpholine (4.14 mL, 37.6 mmol) were dissolved in DMSO (20 mL). Pramipexole dihydrochloride (2.14 g, 7.52 mmol) was added, and the mixture was stirred for 1 h. The solution was diluted with 300 mL 1 M NaOH solution, saturated with NaCl, and extracted with DCM (8×70 mL). The combined organic phases were dried over MgSO₄, the solvent was evaporated under reduced pressure, and the residue purified by RP-HPLC. After lyophilisation 721 mg (1.49 mmol, TFA salt) of the Boc protected derivative were obtained.

MS: m/z 369.2=[M+H]⁺ (MW calculated=368.5 g/mol).

For deprotection the intermediate was dissolved in 3 M methanolic HCl (10 mL), concentrated aqueous HCl (400 μL) were added, and the mixture was agitated for 4 h. The solvent was removed under reduced pressure and the residue was dried in vacuo.

Yield: 490 mg (1.44 mmol, double HCl salt).

MS: m/z 269.1=[M+H]⁺ (MW calculated=268.4 g/mol).

### Synthesis of Intermediate (5b)

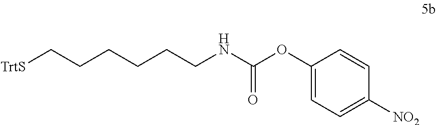

6-Tritylsulfanylhexane-1-amine (1.21 g, 3.22 mmol) and p-nitrophenyl chloroformate (0.78 g, 3.86 mmol) were suspended in dry THF (15 mL). DIEA (841 μL, 4.83 mmol) was added, and the resulting solution was stirred at RT for 2 h. After acidification by addition of acetic acid the solvent was evaporated under reduced pressure, and the residue was purified by RP-HPLC.

Yield: 1.21 g (2.25 mmol)

MS: m/z 563.2=[M+Na]⁺ (MW calculated=540.7 g/mol).

### Synthesis of Linker Pramipexole Conjugate (5c)

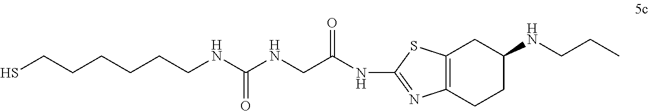

Carbamate 5b (801 mg, 1.48 mmol) was dissolved in DMSO (4.4 mL) and added dropwise to a stirred solution of 5a (490 mg, 1.44 mmol) and DIEA (800 μL, 4.60 mmol) in DMSO (7 mL) within 30 min. The mixture was agitated for 4.5 h at RT. The solution was diluted with 0.5 M NaOH solution (300 mL) and extracted with DCM (6×70 mL). The combined organic phases were dried over MgSO₄, the solvent was evaporated under reduced pressure, and the conjugate was purified by RP-HPLC to obtain 254 mg (0.323 mmol, TFA salt) of the trityl protected intermediate.

MS: m/z 670.3=[M+H]⁺ (MW calculated=670.0 g/mol).

For deprotection the intermediate (248 mg, 0.32 mmol) was incubated in HFIP (6 mL) and TES (240 μL) for 30 min at RT. Volatiles were evaporated, and the residue was purified by RP-HPLC.

Yield: 167 mg (0.31 mmol, TFA salt).

MS: m/z 428.2=[M+H]⁺ (MW calculated=427.6 g/mol).

### EXAMPLE 6

### Synthesis of Linker Pramipexole Conjugate (6)

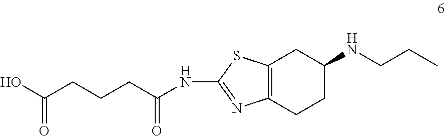

For the synthesis of intermediate 6 glutaric acid anhydride (401 mg, 3.52 mmol), pramipexole dihydrochloride (200 mg, 0.70 mmol), and pyridine (567 μL, 7.04 mmol) were dissolved in dry DMSO (2 mL). The mixture was stirred for 18 hours. The mixture was acidified by addition of acetic acid and 6 was purified by RP-HPLC.

Yield: 191 mg (0.43 mmol, TFA salt).

MS: m/z 326.2=[M+H]⁺ (MW calculated=325.4 g/mol).

### EXAMPLE 7

### Synthesis of OEG-Carrier (7)

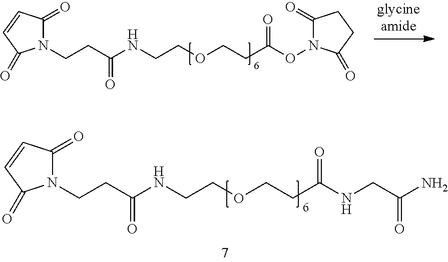

Maleimide-dPEG₆-NHS-ester (75 mg, 0.125 mmol) was dissolved in 7/3 acetonitrile/water (3 mL). 0.5 M phosphate buffer pH 7.0 (300 μL) and glycine amide hydrochloride (41 mg, 0.374 mmol) were added, and the solution was agitated for 30 min at RT. The mixture was diluted by addition of water (3 mL) and 7 was purified by RP-HPLC.

Yield: 54 mg (0.096 mmol).

MS: m/z 561.3=[M+H]⁺ (MW calculated=560.6 g/mol).

### EXAMPLE 8

### Synthesis of OEG-Carrier on Resin (8)

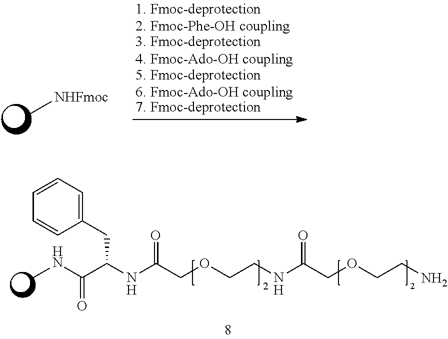

PEG-carrier 8 was synthesized on Sieber amide resin (600 mg, 0.38 mmol) by loading the resin with Fmoc-Phe-OH, Fmoc-deprotection, coupling with Fmoc-8-amino-3,6-dioxa-octanoic acid, Fmoc-deprotection, second coupling with Fmoc-8-amino-3,6-dioxa-octanoic acid and Fmoc-deprotection as depicted above and described in “Materials and Methods”. Product formation was confirmed by MS analysis after cleavage of fmoc protected product (step 6) from a small amount of resin as described in “Materials and Methods”.

MS: m/z 677.3=[M+H]⁺ (MW calculated=676.8 g/mol).

### EXAMPLE 9

### Carrier Linked Pramipexole Prodrugs

### Synthesis of PEG-Pramipexole Conjugates (9a), (9b), (9c), (9d), and (9e)

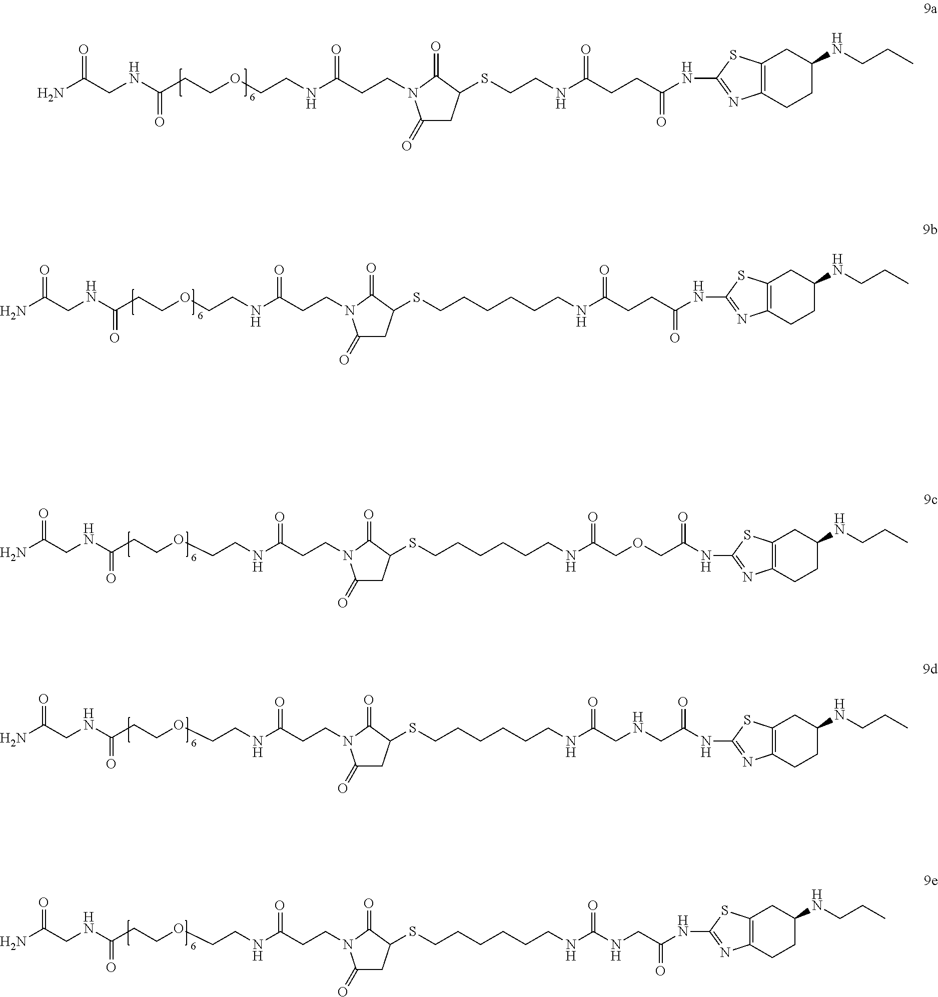

7 (4.5 mg, 0.008 mmol) and 1b (2 mg, 0.004 mmol) were dissolved in 1/1 acetonitrile/water (197 μL). 0.5 M phosphate buffer pH 7.4 (23 μL) was added and the solution agitated for 10 min at RT. The mixture was acidified by addition of acetic acid, diluted with water (200 μL), and 9a was purified by RP-HPLC.

Yield: 3.2 mg (0.003 mmol, TFA salt).

MS: m/z 931.4=[M+H]⁺ (MW calculated=931.2 g/mol).

9b was synthesized as described for 9a except for the use of 2b (1.8 mg, 0.003 mmol) instead of 1b.

Yield: 3.3 mg (0.003 mmol, TFA salt).

MS: m/z 987.5=[M+H]⁺ (MW calculated=987.3 g/mol).

9c was synthesized as described for 9a except for the use of 3b (2 mg, 0.004 mmol) instead of 1b.

Yield: 4.2 mg (0.004 mmol, TFA salt).

MS: m/z 1003.5=[M+H]⁺ (MW calculated=1003.3 g/mol).

9d was synthesized as described for 9a except for the use of 4b (2 mg, 0.003 mmol) instead of 1b.

Yield: 1.8 mg (0.0015 mmol, double TFA salt).

MS: m/z 1002.5=[M+H]⁺ (MW calculated=1002.3 g/mol).

9e was synthesized as described for 9a except for the use of 5c (2 mg, 0.004 mmol) instead of 1b.

Yield: 3.4 mg (0.003 mmol, TFA salt).

MS: m/z 988.5=[M+H]⁺ (MW calculated=988.2 g/mol).

### EXAMPLE 11

### Carrier Linked Pramipexole Prodrug

### Synthesis of OEG-Linker Pramipexole Conjugate (11)

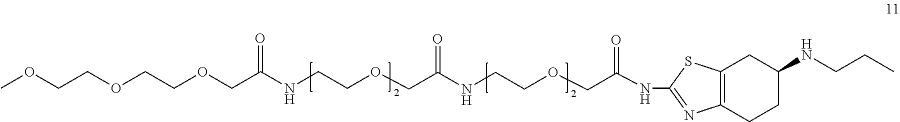

### Synthesis of OEG-carrier 10

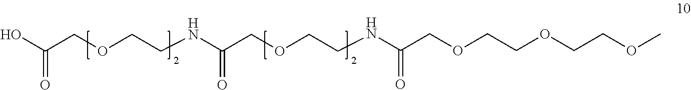

OEG-carrier 10 was synthesized on 2-chlorotrityl chloride resin (362 mg, 0.365 mmol) by loading the resin with Fmoc-Ado-OH, Fmoc deprotection, coupling with Fmoc-Ado-OH, Fmoc deprotection, coupling with 2-(2-(2-methoxyethoxy)ethoxy)acetic acid, and cleavage from the resin as described in “Materials and Methods”. Crude 10 was used in the next step without further purification.

Yield: 141 mg (0.301 mmol).

MS: m/z 469.2=[M+H]⁺ (MW calculated=468.5 g/mol).

### Synthesis of OEG Linker Pramipexole Conjugate (11)

For the synthesis of conjugate 11 PEG-carrier 10 (19.6 mg, 0.041 mmol), PyBOP (26 mg, 0.050 mmol) and N-methyl morpholine (37 μL, 0.335 mmol) were dissolved in DMSO (300 μL). Pramipexole dihydrochloride (35 mg, 0.122 mmol) was added and the mixture was agitated at RT for 24 hours. 11 was purified by RP-HPLC.

Yield: 14.6 mg (0.019 mmol, TFA salt).

MS: m/z 662.3=[M+H]⁺ (MW calculated=661.8 g/mol).

### EXAMPLE 12

### Synthesis of Backbone Reagents (12g) and (12h)

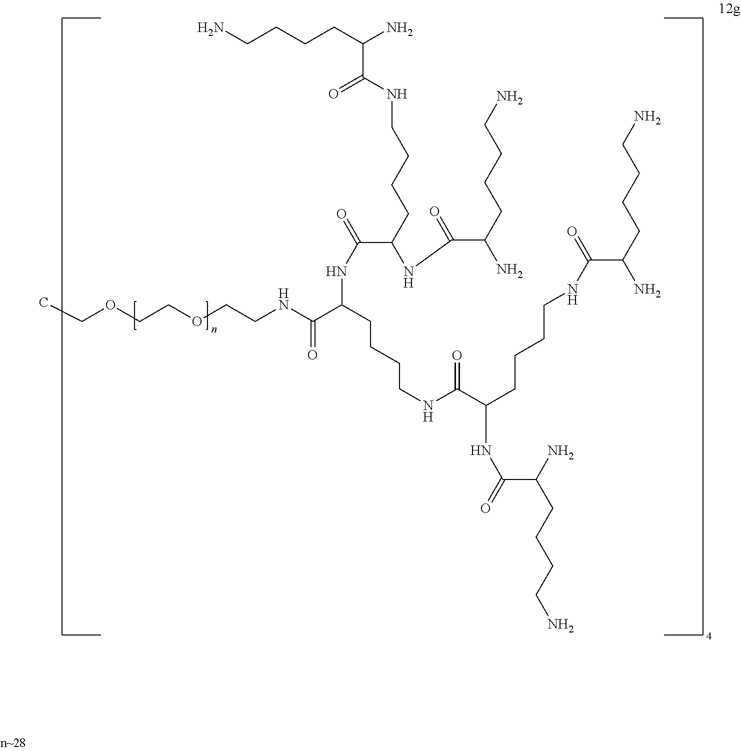

Backbone reagent 12g was synthesized from Amino 4-arm PEG5000 12a according to the following scheme:

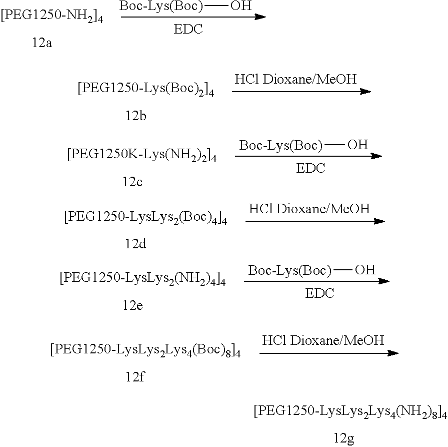

For synthesis of compound 12b, 4-Arm-PEG5000 tetraamine 12a (MW ca. 5200 g/mol, 5.20 g, 1.00 mmol, HCl salt) was dissolved in 20 mL of DMSO (anhydrous). Boc-Lys(Boc)-OH (2.17 g, 6.25 mmol) in 5 mL of DMSO (anhydrous), EDC HCl (1.15 g, 6.00 mmol), HOBt.H₂O (0.96 g, 6.25 mmol), and collidine (5.20 mL, 40 mmol) were added. The reaction mixture was stirred for 30 min at RT.

The reaction mixture was diluted with 1200 mL of dichloromethane and washed with 600 mL of 0.1 N H₂SO₄ (2×), brine (1×), 0.1 M NaOH (2×), and 1/1 (v/v) brine/water (4×). Aqueous layers were reextracted with 500 mL of DCM. Organic phases were dried over Na₂SO₄, filtered and evaporated to give 6.3 g of crude product 12b as colorless oil. Compound 12b was purified by RP-HPLC.

Yield 3.85 g (59%) colorless glassy product 12b.

MS: m/z 1294.4=[M+5H]⁵⁺ (m/z of [M+5H]⁵⁺ calculated for a 4-Arm-PEG containing a total of 107 ethylene glycol units=1294.6).

Compound 12c was obtained by stirring of 3.40 g of compound 12b (0.521 mmol) in 5 mL of methanol and 9 mL of 4 N HCl in dioxane at RT for 15 min. Volatiles were removed in vacuo. The product was used in the next step without further purification.

MS: m/z 1151.9=[M+5H]⁵⁺ (m/z calculated=1152.0).

For synthesis of compound 12d, 3.26 g of compound 12c (0.54 mmol) were dissolved in 15 mL of DMSO (anhydrous). 2.99 g Boc-Lys(Boc)-OH (8.64 mmol) in 15 mL DMSO (anhydrous), 1.55 g EDC HCl (8.1 mmol), 1.24 g HOBt.H₂O (8.1 mmol), and 5.62 mL of collidine (43 mmol) were added. The reaction mixture was stirred for 30 min at RT. Reaction mixture was diluted with 800 mL DCM and washed with 400 mL of 0.1 N H₂SO₄ (2×), brine (1×), 0.1 M NaOH (2×), and 1/1 (v/v) brine/water (4×). Aqueous layers were reextracted with 800 mL of DCM. Organic phases were dried with Na₂SO₄, filtered and evaporated to give a glassy crude product.

Product was dissolved in DCM and precipitated with cooled (−18° C.) diethylether. This procedure was repeated twice and the precipitate was dried in vacuo.

Yield: 4.01 g (89%) colorless glassy product 12d, which was used in the next step without further purification.

MS: m/z 1405.4=[M+6H]⁶⁺ (m/z calculated=1405.4).

Compound 12e was obtained by stirring a solution of compound 12d (3.96 g, 0.47 mmol) in 7 mL of methanol and 20 mL of 4 N HCl in dioxane at RT for 15 min. Volatiles were removed in vacuo. The product was used in the next step without further purification.

MS: m/z 969.6=[M+7H]⁷⁺ (m/z calculated=969.7).

For the synthesis of compound 12f, compound 12e (3.55 g, 0.48 mmol) was dissolved in 20 mL of DMSO (anhydrous). Boc-Lys(Boc)-OH (5.32 g, 15.4 mmol) in 18.8 mL of DMSO (anhydrous), EDC HCl (2.76 g, 14.4 mmol), HOBt.H₂O (2.20 g, 14.4 mmol), and 10.0 mL of collidine (76.8 mmol) were added. The reaction mixture was stirred for 60 min at RT.

The reaction mixture was diluted with 800 mL of DCM and washed with 400 mL of 0.1 N H₂SO₄ (2×), brine (1×), 0.1 M NaOH (2×), and 1/1 (v/v) brine/water (4×). Aqueous layers were reextracted with 800 mL of DCM. Organic phases were dried over Na₂SO₄, filtered and evaporated to give crude product 12f as colorless oil.

Product was dissolved in DCM and precipitated with cooled (−18° C.) diethylther. This step was repeated twice and the precipitate was dried in vacuo.

Yield 4.72 g (82%) colourless glassy product if which was used in the next step without further purification.

MS: m/z 1505.3=[M+8H]⁸⁺ (m/z calculated=1505.4).

Backbone reagent 12g was obtained by stirring a solution of compound 12f (MW ca 12035 g/mol, 4.72 g, 0.39 mmol) in 20 mL of methanol and 40 mL of 4 N HCl in dioxane at RT for 30 min. Volatiles were removed in vacuo.

Yield 3.91 g (100%), glassy product backbone reagent 12g.

MS: m/z 977.2=[M+9H]⁹⁺ (m/z calculated=977.4).

### Synthesis of Backbone Reagent 12h

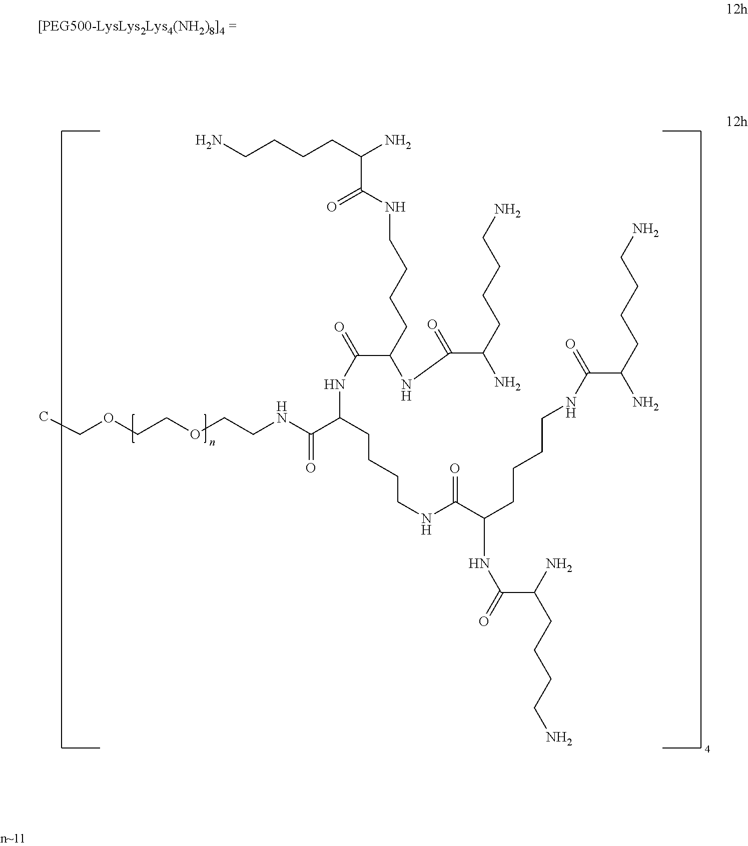

Backbone reagent 12h was synthesized as described for 12g except for the use of 4-arm PEG2000 instead of 4-arm PEG5000.

MS: m/z 719.4=[M+8H]⁸⁺ (calculated=719.5).

### EXAMPLE 13

### Synthesis of Crosslinker Reagents (13d), (13e), and (13f)

Crosslinker reagent 13d was prepared from adipic acid mono benzyl ester (English, Arthur R. et al., Journal of Medicinal Chemistry, 1990, 33(1), 344-347) and PEG2000 according to the following scheme:

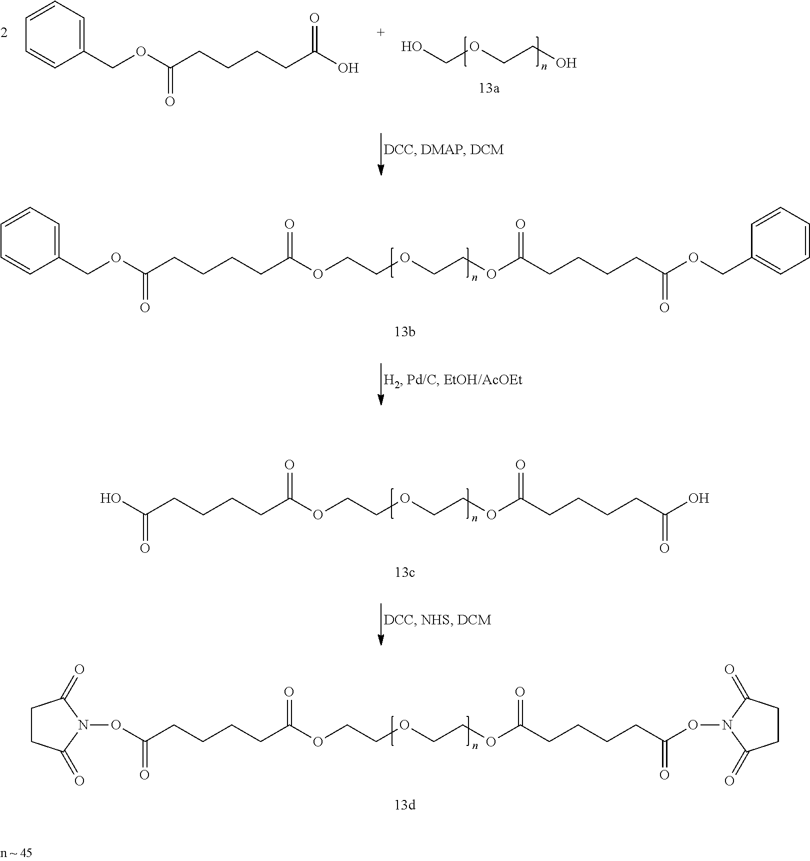

A solution of PEG2000 (13a) (11.0 g, 5.5 mmol) and benzyl adipate half-ester (4.8 g, 20.6 mmol) in dichloromethane (90.0 mL) was cooled to 0° C. Dicyclohexylcarbodiimide (4.47 g, 21.7 mmol) was added followed by a catalytic amount of DMAP (5 mg) and the solution was stirred and allowed to reach RT overnight (12h). The flask was stored at +4° C. for 5 h. The solid was filtered and the solvent completely removed by destillation in vacuo. The residue was dissolved in 1000 mL 1/1(v/v) ether/ethyl acetate and stored at RT for 2 hours while a small amount of a flaky solid was formed. The solid was removed by filtration through a pad of Celite®. The solution was stored in a tightly closed flask at −30° C. in the freezer for 12 h until crystallisation was complete. The crystalline product was filtered through a glass frit and washed with cooled ether (−30° C.). The filter cake was dried in vacuo. Yield: 11.6 g (86%) 13b as a colorless solid. The product was used without further purification in the next step.

MS: m/z 813.1=[M+3H]³⁺ (m/z of [M+3H]³⁺ calculated for n=44: 813.3)

In a 500 mL glass autoclave PEG2000-bis-adipic acid-bis-benzyl ester 13b (13.3 g, 5.5 mmol) was dissolved in ethyl acetate (180 mL) and 10% Palladium on charcoal (0.4 g) was added. The solution was hydrogenated at 6 bar, 40° C. until consumption of hydrogen had ceased (5-12 h). Catalyst was removed by filtration through a pad of Celite® and the solvent was evaporated in vacuo. Yield: 12.3 g (quantitative) 13c as yellowish oil. The product was used without further purification in the next step.

MS: m/z 753.1=[M+3H]³⁺ (calculated=753.2)

A solution of PEG2000-bis-adipic acid half ester 13c (9.43 g, 4.18 mmol), N-hydroxysuccinimide (1.92 g, 16.7 mmol) and DCC (3.44 g, 16.7 mmol) in 75 mL of DCM (anhydrous) was stirred over night at RT. The reaction mixture was cooled to 0° C. and precipitate was filtered off. DCM was evaporated and the residue was recrystallized from THF.

Yield: 8.73 g (85%) crosslinker reagent 13d as colorless solid.

MS: m/z 817.8=[M+3H]³⁺ (calculated=817.9).

### Synthesis of 13e

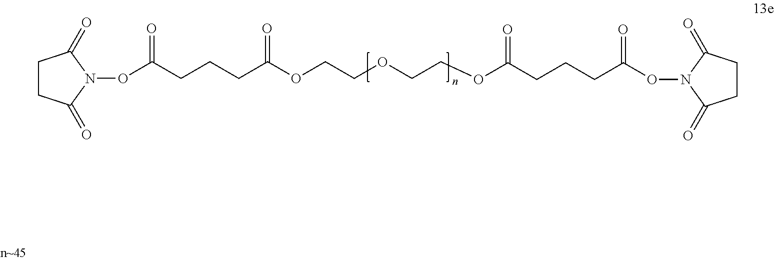

13e was synthesized as described for 13d except for the use of glutaric acid instead of adipic acid

MS: m/z 764.4=[M+3H]³⁺ (calculated=764.5).

### Synthesis of 13f

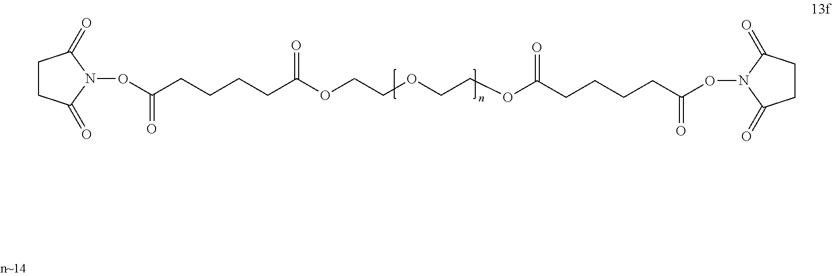

13f was synthesized as described for 13d except for the use of PEG600 instead of PEG2000

MS: m/z 997.5=[M+H]⁺ (calculated=997.8)

### Synthesis of 13g

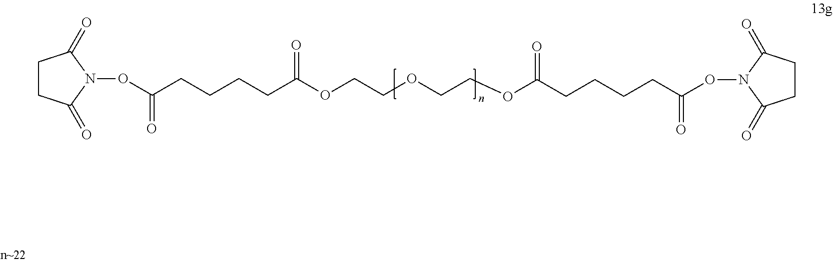

13g was synthesized as described for 13d except for the use of PEG1000 instead of PEG2000

MS: m/z 697.4=[M+2H]² (calculated=697.3)

### EXAMPLE 14

### Preparation of Hydrogel Beads (14a), (14b), and (14c) Containing Free Amino Groups

A solution of 300 mg 12g and 900 mg 13d in 10.8 mL DMSO was added to a solution of 100 mg Arlacel P135 in 60 mL heptane. The mixture was stirred at 700 rpm with a custom metal stirrer for 10 min at RT to form a suspension. 1.1 mL N,N,N′,N′-tertramethylethylene diamine (TMEDA) was added to effect polymerization. After 2 h, the stirrer speed was reduced to 400 rpm and the mixture was stirred for additional 16 h. 1.6 mL of acetic acid were added and then after 10 min 50 mL of water were added. After 5 min, the stirrer was stopped and the aqueous phase was drained.

For bead size fractionation, the water-hydrogel suspension was wet-sieved on 75, 50, 40, 32 and 20 μm steel sieves. Bead fractions that were retained on the 32, 40, and 50 μm sieves were pooled and washed 3 times with water, 10 times with ethanol and dried for 16 h at 0.1 mbar to give 14a as a white powder.

14b was prepared as described for 14a except for the use of 322 mg 12h, 350 mg 13f, 2.9 ml DMSO, 1.6 ml TMEDA, 2.4 ml acetic acid and a stirring speed of 1000 rpm.

14c was prepared as described for 14a except for the use of 300 mg 12g, 810 mg 13e, 6.3 ml DMSO, 1.1 ml TMEDA, 1.6 ml acetic acid and a stirring speed of 1000 rpm.

14d was prepared as described for 14a except for the use of 900 mg 12g, 886 mg 13g, 300 mg Arlacel P135, 6.7 ml DMSO, 3.2 ml TMEDA, 5 ml acetic acid and a stirring speed of 1500 rpm.

14e was prepared as described for 14a except for the use of 900 mg 12g, 1476 mg 13d, 320 mg Arlacel P135, 8.0 ml DMSO, 3.2 ml TMEDA, 5 ml acetic acid and a stirring speed of 1500 rpm.

Content of amino groups in hydrogel beads was determined as described in “Materials and Methods”

| | amine content |
|---|---|
| | [mmol/g] |
| 14c | 0.84-0.93 |
| 14d | 0.99-1.18 |
| 14e | 0.82-0.93 |

### EXAMPLE 15

### Preparation of Maleimide Functionalized Hydrogel Beads (15a) and (15b) and Determination of Maleimide Substitution

A solution of 600 mg Mal-dPEG₆-NHS (1.0 mmol) in 4.5 mL 2/1 (v/v) acetonitrile/water was added to 200 mg dry hydrogel beads 14a. 500 μL sodium phosphate buffer (pH 7.4, 0.5 M) was added and the suspension was agitated for 30 min at RT. Beads 15a were washed five times each with 2/1 (v/v) acetonitrile/water, methanol and 1/1/0.001 (v/v/v/) acetonitrile/water/TFA.

For determination of maleimide content, an aliquot of hydrogel beads 15a was lyophilized and weighed out. Another aliquot of hydrogel beads 15a was reacted with excess mercaptoethanol (in 50 mM sodium phosphate buffer, 30 min at RT), and mercaptoethanol consumption was detected by Ellman test (Ellman, G. L. et al., Biochem. Pharmacol., 1961, 7, 88-95). Maleimide content was determined to be 0.27 mmol/g dry hydrogel.

15b was prepared by coupling 3-Maleimidopropionic acid to 14b as described in “Materials and Methods”. After coupling, beads 15b were washed five times each with 2/1 (v/v) acetonitrile/water, methanol and 1/1/0.001 (v/v/v/) acetonitrile/water/TFA. Maleimide content 15b: 0.9 mmol/g

### EXAMPLE 16

### Carrier Linked Pramipexole Prodrugs

### Synthesis of Hydrogel-Linker-Pramipexole Conjugate (16a) and (16b)

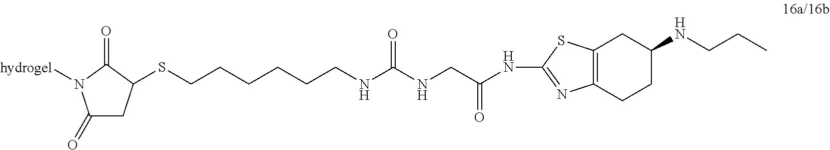

Maleimide-derivatized hydrogel microparticles 15a (100 μL. loading 30 μmol/mL, 3 mmol) were reacted with compound 5c (2.3 mg, 4.3 μmol) in 1/1 acetonitrile/water (420 μL) and 0.5 M phosphate buffer pH 7.4 (52 μL) for 10 min at RT. The hydrogel was washed 20 times with 1/1 acetonitrile/water. Remaining maleimides where reacted with 2-mercaptoethanol (34 μL, 0.48 mmol) in 1/1 acetonitrile/water (3 mL) and 0.5 M phosphate buffer pH 7.4 (0.4 mL) for 10 min at RT. The loaded hydrogel was washed 20 times with 1/1 acetonitrile/water.

Pramipexole loading 16a: 27 mg/g

High loaded pramipexole linker hydrogel 16b was prepared as described above except for the use of 88 mg 5c and 100 mg 15b.

Pramipexole loading 16b: 152 mg/g

### EXAMPLE 17

### Synthesis of Glycyl-Pramipexole(Boc) (17b)

### Synthesis of Intermediate (17a)

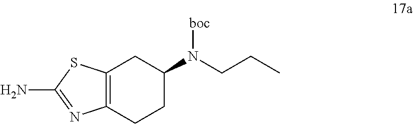

Derivative 17a was synthesized by dissolving pramipexole dihydrochloride (400 mg, 1.41 mmol) and Boc₂O (307 mg, 1.41 mmol) in DMSO (5 mL). DIEA (735 μL, 4.22 mmol) was added and the solution was stirred for three hours at rt. The product was purified by RP-HPLC.

Yield: 422 mg (0.99 mmol, TFA salt).

MS: m/z 312.2=[M+H]⁺ (MW calculated=311.5 g/mol).

### Synthesis of Glycyl-Pramipexole(boc) (17b)

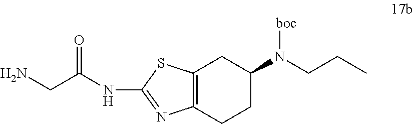

17a (50 mg, 0.118 mmol), Fmoc-Gly-OH (52 mg, 0.176 mmol) and PyBOP (104 mg, 0.200 mmol) were dissolved in DMSO (200 μL). DIEA (90 μL, 0.517 mmol) was added and the solution was agitated for 15 hours. The Fmoc-protected intermediate was purified by RP-HPLC.

Yield: 72 mg (0.12 mmol).

MS: m/z 591.3=[M+H]⁺ (MW calculated=590.8 g/mol).

For deprotection the intermediate was dissolved in piperidine/DBU/DMF (2/2/96) and stirred for 30 min at RT. Product 17b was purified by RP-HPLC.

Yield: 30.6 mg (0.06 mmol, TFA salt).

MS: m/z 369.2=[M+H]⁺ (MW calculated=368.5 g/mol).

### EXAMPLE 18

### Synthesis of Linker Pramipexole Conjugate (18)

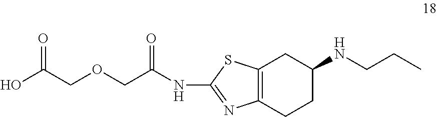

Pramipexole dihydrochloride (200 mg, 0.704 mmol), diglycol anhydride (82 mg, 0.704 mmol) and pyridine (199 mL, 2.462 mmol) were dissolved in DMSO (2 mL) and stirred at RT for three hours. Product 18 was purified by RP-HPLC.

Yield: 107.7 mg (0.244 mmol, TFA salt).

MS: m/z 328.1=[M+H]⁺ (MW calculated=327.4 g/mol).

### EXAMPLE 19

### Synthesis of OEG-Carrier-Resin (19b) and (19c)

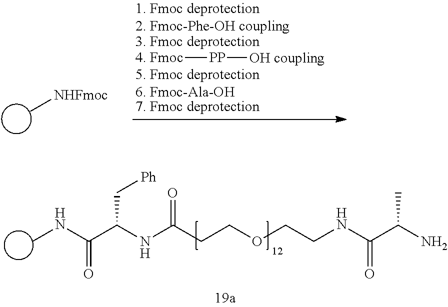

OEG-carrier-resin 19a was synthesized on Sieber amide resin (242 mg, 0.167 mmol) by loading resin with Fmoc-Phe-OH, followed by a sequence of Fmoc deprotection and coupling of the Fmoc-protected amino acids Fmoc-PP-OH and Fmoc-Ala-OH as described in “Materials and Methods”.

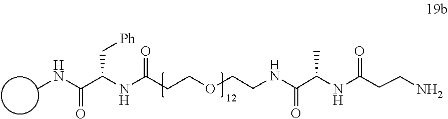

19b was synthesized by coupling Fmoc-beta-Ala-OH to loaded resin 19a, and subsequent Fmoc deprotection according to procedures in “Materials and Methods”.

Product identity was confirmed by cleavage of a small amount of resin as described in “Materials and Methods”.

MS: m/z 906.5=[M+H]⁺ (calculated=906.5 g/mol).

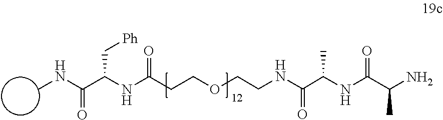

19c was synthesized by coupling of Fmoc-Ala-OH to loaded resin 19a, and subsequent Fmoc deprotection according to procedures in “Materials and Methods”.

Product formation was confirmed by MS analysis after cleavage of product from a small amount of resin as described in “Materials and Methods”.

MS: m/z 906.5=[M+H]⁺ (MW calculated=906.1 g/mol).

### EXAMPLE 20

### Carrier Linked Pramipexole Prodrug

### Synthesis of OEG-Linker Pramipexole Conjugate (20)

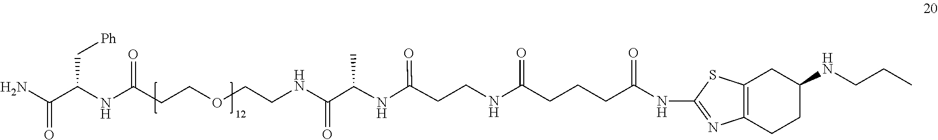

OEG-linker pramipexole conjugate 20 was synthesized by coupling 6 (5.4 mg, 0.012 mmol) to OEG-carrier resin 19b (25 mg, 0.012 mmol) with PyBOP (7.5 mg, 0.014 mmol) and DIEA (7.1 μL, 0.041 mmol) in DMF (0.8 mL). Product 20 was cleaved from the resin as described in “Materials and Methods” and purified by RP-HPLC.

Yield: 3.9 mg (0.003 mmol, TFA salt).

MS: m/z 1213.7=[M+H]⁺ (MW calculated=1213.5 g/mol).

### EXAMPLE 21

### Carrier Linkend Pramipexole Prodrug

### Synthesis of OEG-Linker Pramipexole Conjugate (21)

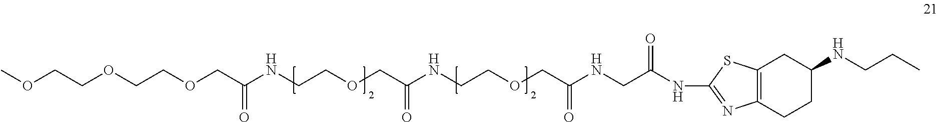

11a (18.4 mg, 0.039 mmol) and 5a (27 mg, 0.079 mmol) were dissolved in DMSO (300 μL). DIEA (100 μL, 0.576 mmol) and PyBOP (45 mg, 0.079 mmol) were added and the solution was agitated for 1 hour at RT. Product 21 was purified by RP-HPLC.

Yield: 32.9 mg (0.039 mmol, TFA salt).

MS: m/z 719.4=[M+H]⁺ (MW calculated=718.9 g/mol).

### EXAMPLE 22

### Carrier Linked Pramipexole Prodrug

### Synthesis of OEG-Linker Pramipexole Conjugate (22b)

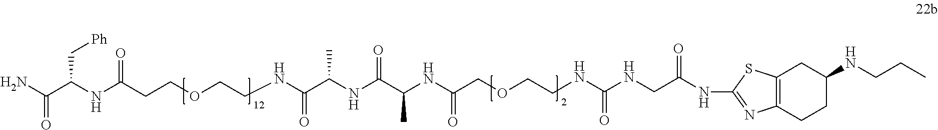

### Synthesis of Pramipexole Linker Intermediate (22a)

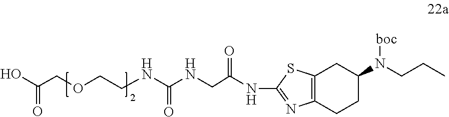

Intermediate 22a was synthesized on 2-Chlorotrityl chloride resin by loading the resin with Fmoc-Ado-OH, Fmoc deprotection, urea formation with 17b and cleavage from the resin as described in “Materials and Methods”. Product 22a was purified by RP-HPLC.

Yield: 15.3 mg (0.027 mmol).

MS: m/z 558.3=[M+H]⁺ (MW calculated=557.7 g/mol).

### Synthesis of OEG-Linker Pramipexole Conjugate 22b

Resin 19b (36 mg, 0.018 mmol) was reacted with 22a (10 mg, 0.018 mmol), PyBOP (11.2 mg, 0.022 mmol) and DIEA (7.5 μL, 0.043 mmol) in DMF (400 μL). Product 22b was obtained by cleavage from resin as described in “Materials and Methods” and RP-HPLC purification.

Yield: 17.3 mg (0.012 mmol, TFA salt).

MS: m/z 1345.7=[M+H]⁺ (MW calculated=1345.6 g/mol).

### EXAMPLE 23

### Carrier Linked Pramipexole Prodrug)

### Synthesis of OEG-Linker Pramipexole Conjugate (23)

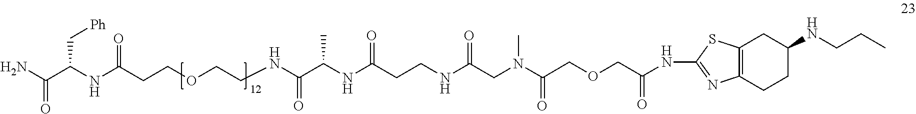

23 was synthesized by coupling of resin 19b (20 mg, 0.010 mmol) with Fmoc-sarcosine, Fmoc deprotection, coupling with 18 and cleavage from the resin as described in “Materials and Methods”. Product 23 was purified by RP-HPLC.

Yield: 7 mg (0.005 mmol, TFA salt).

MS: m/z 1286.7=[M+H]⁺ (MW calculated=1286.6 g/mol).

### EXAMPLE 24

### Carrier Linked Pramipexole Prodrug

### Synthesis of OEG-Linker Pramipexole Conjugate (24b)

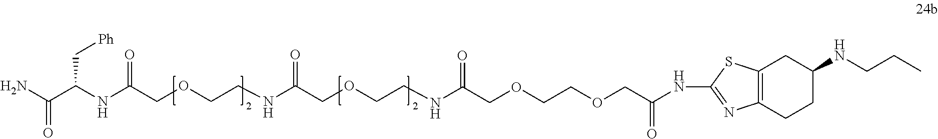

### Synthesis of Pramipexole Intermediate 24a

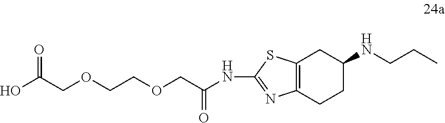

Intermediate 24a was synthesized by reacting pramipexole dihydrochloride (50 mg, 0.176 mmol) with 3,6-dioxa-suberic acid (31 mg, 0.176 mmol) by addition of PyBOP (110 mg, 0.211 mmol) and N-methyl morpholine (854, 0.774 mmol) in DMSO (800 μL). The solution was stirred for one hour at RT and the product was purified by PR-HPLC.

Yield: 22 mg (0.045 mmol, TFA salt).

MS: m/z 372.2=[M+H]⁺ (MW calculated=371.5 g/mol).

### Synthesis of OEG Linker Pramipexole Conjugate 24b

OEG-carrier-resin 8 (19 mg, 0.009 mmol substitution) was coupled with 24a (11 mg, 0.023 mmol) with PyBOP (14 mg, 0.028 mmol) and DIEA (13 μL, 0.074 mmol) in DMF (400 μL). Product 24b was obtained by cleavage from the resin as described in “Materials and Methods” and RP-HPLC purification.

Yield: 6.1 mg (0.007 mmol, TFA salt).

MS: m/z 808.4=[M+H]⁺ (MW calculated=808.0 g/mol).

### EXAMPLE 25

### Synthesis of Pramipexole Linker Conjugate (25b)

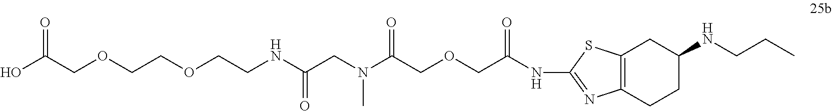

### Synthesis of Linker Intermediate-Resin 25a

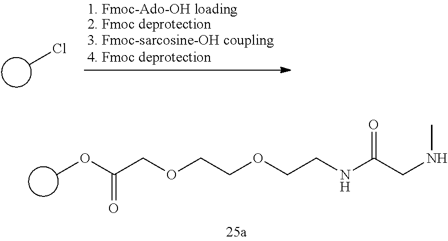

Intermediate 25a was synthesized on 2-Chlorotrityl chloride resin by loading the resin with Fmoc-Ado-OH, Fmoc deprotection, coupling with Fmoc-sarcosine and Fmoc deprotection and as depicted above and described in “Materials and Methods”.

### Synthesis of Conjugate (25b)

Linker intermediate-resin 25a (1.359 g, 0.433 mmol) was agitated with diglycol anhydride (81 mg, 0.701 mmol) and pyridine (141 μL, 1.76 mmol) in DMF (1.6 mL) at RT for two hours. The resin was washed with DMF (10×) and DCM (10×) and dried under reduced pressure. A solution of 17a (276 mg, 0.650 mmol), PyBOP (270 mg, 0.520 mmol) and DIEA (339 μL, 1.949 mmol) in DMSO (3 mL) was added and agitated for two hours. The resin was washed with DMF (10×) and DCM (10×) and the boc-protected intermediate was cleaved from the resin as described in “Materials and Methods”. Volatiles were removed under reduced pressure, the residue was taken up in 1/1 TFA/DCM and stirred for 30 min. Product 25b was purified by RP-HPLC.

Yield: 94 mg (0.162 mmol, HCl salt).

MS: m/z 544.2=[M+H]⁺ (MW calculated=543.6 g/mol).

### EXAMPLE 26

### Carrier Linked Pramipexole Prodrug

### Synthesis of Hydrogel-Linker-Pramipexole Conjugate (26)

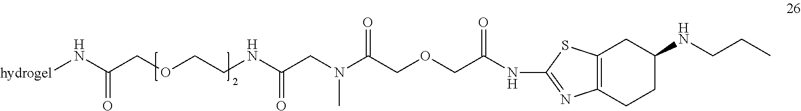

Hydrogel beads 14d containing free amino groups (320.5 mg, loading 0.99 mmol/g, 317.3 μmol) were reacted with compound 25b (460 mg, 793 μmol), PyBOP (462 mg, 888 μmol) and collidine (210 μL, 1.587 mmol) in DMF (3.5 mL) for 2.5 hours at RT. The loaded hydrogel was washed 10 times with DMF, 10 times with 1/1 acetonitrile/water, 8 times with phosphate buffer pH 7.4 and again 10 times with 1/1 acetonitrile/water. Pramipexole loading: 125 mg/g

### EXAMPLE 27

### Release Kinetics In Vitro

Release of drug molecule was effected by hydrolysis in buffer at pH 7.4 and 37° C.

9a, 9b, 9c, 9d, 9e, 11b, 20, 21, 22b, 23, 24b, 33, and 35 respectively, were dissolved in buffer (60 mM sodium phosphate, 3 mM EDTA, 0.01% Tween20, pH 7.4), solution was filtered through a 0.2 μm filter and incubated at 37° C. Samples were taken at time intervals and analyzed by RP-HPLC at 263 nm and 280 nm and ESI-MS. UV-signals correlating to linker conjugate molecules were integrated and plotted against incubation time. Curve-fitting software was applied to estimate the corresponding half time of release

16a, 16b, 26, 30, and 31 respectively, were suspended in buffer (60 mM sodium phosphate, 3 mM EDTA, 0.01% Tween20, pH 7.4) and incubated at 37° C. At time intervals samples were vortexed and centrifuged. Aliquots (10-50 μL) were taken from the supernatant solution, and analyzed by measurement of the absorption of released drug at 263 nm. Calculated amounts of released drug were plotted against incubation time. Alternatively, amount of released pramipexole was determined by size exclusion chromatography as described in “Materials and Methods”.

Curve-fitting software was applied to estimate the corresponding half time of release.

FIG. 1 additionally depicts the in vitro release kinetic of the carrier linked pramipexole prodrug of example 16a. The x-axis shows the time [unit: days].

| Compound | t_{1/2} buffer A (pH 7.4) | |
|---|---|---|
| 9a | 1.2 | d |
| 9b | 3.7 | d |
| 9c | 1.3 | d |
| 9d | 1.1 | d |
| 9e | 4.3 | d |
| 11 | 19 | d |
| 16a | 5.1 | d |
| 16b | 15 | d |
| 20 | 120 | d |
| 21 | 22 | d |
| 22b | 3.4 | d |
| 23 | 13 | d |
| 24b | 20 | d |
| 26 | 14 | d |
| 30 | 10 | d |
| 31 | 14 | d |
| 33 | 12 | d |
| 35 | 51 | d |

### EXAMPLE 28

### Further Hydrogel Modification

### Synthesis of Ado-Modified Hydrogels (28a), (28b), and (28c) and Lys-Modified Hydrogel (28d)

### Synthesis of Ado-Modified Hydrogel (28a, 28b, 28c):

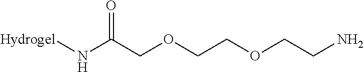

Hydrogel 14a, 14b, and 14c, respectively, in a syringe equipped with a polypropylene frit was washed with 1% diisopropylethylamine solution in DMF and ten times with DMF. Fmoc-Ado-OH coupling was then performed by agitating 14a, 14b, and 14c, respectively, with 3.5 eq of Fmoc-Ado-OH, 3.5 eq of PyBOP and 8.75 eq of DIPEA in DMF (using 0.2 mmol/mL fmoc-Ado-OH concentration). After 45 min, hydrogel was washed with DMF (10 times), then with DCM (10 times).

Fmoc-deprotection was achieved by agitating the hydrogel two times with a 96/2/2 DMF/piperidine/DBU (v/v) solution for 5 min each. 28a, 28b, and 28c, respectively, was then washed with DMF (10 times) and ethanol (10 times) and finally dried in vacuo.

### Synthesis of Lys-Modified Hydrogel (28d, 28e):

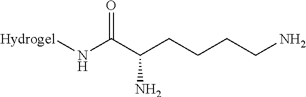

Hydrogel 14a in a syringe equipped with a polypropylene frit was washed with 1% diisopropylethylamine solution in DMF and ten times with DMF.

Fmoc-Lys(Fmoc)-OH coupling was then performed by agitating 14a with 3.5 eq of Fmoc-Lys(Fmoc)-OH, 3.5 eq of PyBOP and 8.75 eq of DIPEA in DMF (using 0.2 mmol/mL Fmoc-Lys-OH concentration). After 45 min, hydrogel was washed with DMF (10 times), then with DCM (10 times).

Fmoc-deprotection was achieved by agitating the hydrogel two times with a 96/2/2 DMF/piperidine/DBU (v/v) solution for 5 min each. 28d was then washed with DMF (10 times) and ethanol (10 times) and finally dried in vacuo.

Hydrogel 28e was obtained as described for 28d except for the use of hydrogel 14e as starting material.

### EXAMPLE 29

### Synthesis of Pramipexole Linker Conjugate (29)

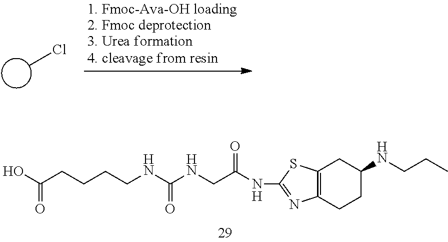

Intermediate 29 was synthesized on 420 mg 2-Chlorotrityl chloride resin. Resin loading with Fmoc-Ava-OH, Fmoc deprotection, urea formation by using 17b and cleavage of 29 from resin was performed as described in “Material and Methods”. Boc deprotection was achieved by dissolving crude product in TFA and stirring for 10 min at RT. TFA was removed under a stream of nitrogen and product 29 was purified by RP-HPLC.

Yield: 160 mg (0.258 mmol, HCl salt).

MS: m/z 412.2=[M+H]⁺ (MW calculated=411.5 g/mol).

### EXAMPLE 30

### Carrier Linked Pramipexole Prodrug

### Synthesis of Hydrogel-Linker-Pramipexole Conjugate (30)

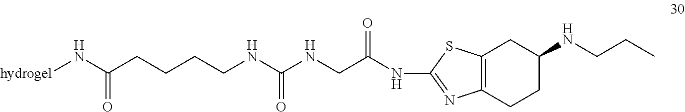

Hydrogel beads 14d containing free amino groups (145 mg, loading 1.18 mmol/g, 298 μmol) were reacted with compound 29 (324 mg, 732 μmol), PyBOP (421 mg, 809 μmol) and collidine (191 μL, 1.45 mmol) in DMF (3 mL) for 2.5 hours at RT. The loaded hydrogel was washed 10 times with DMF, 10 times with 1/1 acetonitrile/water, 8 times with phosphate buffer pH 7.4 and again 10 times with 1/1 acetonitrile/water. Pramipexole loading: 148 mg/g

### EXAMPLE 31

### Carrier Linked Pramipexole Prodrug

### Synthesis of Hydrogel-Linker-Pramipexole Conjugate (31)

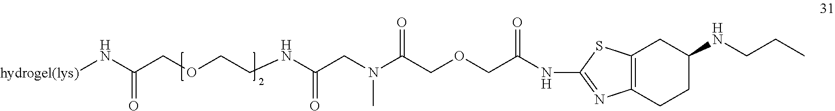

Hydrogel beads 28e containing free amino groups (669 mg, loading 1.48 mmol/g, 0.99 μmol) were reacted with compound 25b (1.14 g, 1.99 mmol), PyBOP (1.16 mg, 2.23 μmol) and collidine (525 μA, 3.98 mmol) in DMF (9 mL) for 2.5 hours at RT. The loaded hydrogel was washed 10 times with DMF, 10 times with 1/1 acetonitrile/water, 8 times with phosphate buffer pH 7.4 and again 10 times with 1/1 acetonitrile/water. Pramipexole loading: 110 mg/g

### EXAMPLE 32

### Synthesis of Linker Pramipexol Conjugate (32c)

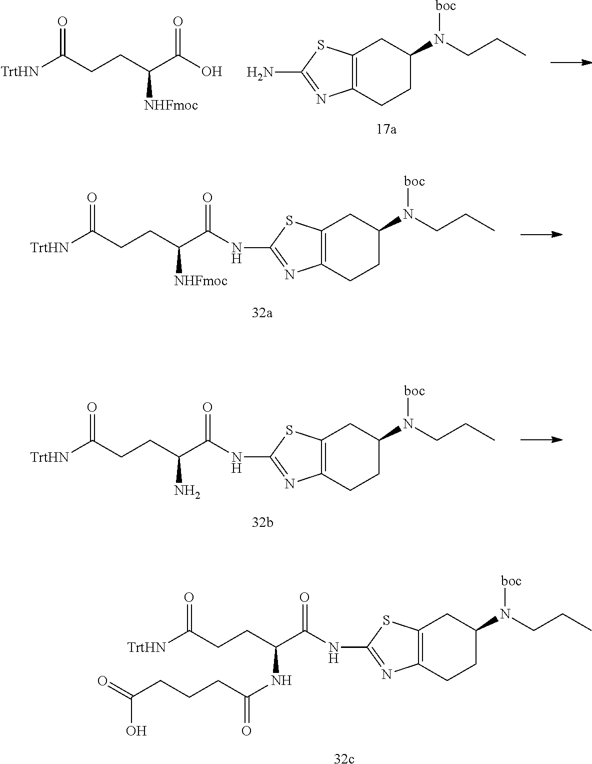

### Synthesis of Pramipexole Intermediate 32a

Intermediate 32a was synthesized by reacting boc-pramipexole 17a (150 mg, 0.482 mmol) with Fmoc-Gln(Trt)-OH (353 mg, 0.482 mmol) by addition of PyBOP (376 mg, 0.722 mmol) and DIPEA (1684, 0.963 mmol) in DCM (2 mL). The solution was stirred for 3 h at RT. Volatiles were removed in vacuo and the product was purified by PR-HPLC.

Yield: 417 mg (0.461 mmol).

MS: m/z 926.4=[M+Na]⁺ (calculated=904.2 g/mol).

### Synthesis of Pramipexole Intermediate 32b

For Fmoc deprotection 32a (417 mg, 0.461 mmol) was dissolved in 3 mL piperidine/DBU/DMF (2/2/96) and stirred for 60 min at RT. Product 32b was purified by RP-HPLC.

Yield: 151 mg (0.192 mmol, TFA salt).

MS: m/z 682.3=[M+H]⁺ (calculated=681.9 g/mol).

### Synthesis of Pramipexole Intermediate 32c

Intermediate 32c was synthesized by reacting boc-pramipexole 32b (124 mg, 0.156 mmol) with glutaric anhydride (27 mg, 0.234 mmol) and pyridine (138 μA, 1.71 mmol) in DCM (2 mL). The solution was stirred for 3 h at RT. Volatiles were removed in vacuo and 32c was purified by PR-HPLC.

Yield: 89 mg (0.112 mmol).

MS: m/z 796.4=[M+H]⁺ (calculated=796.0 g/mol).

### EXAMPLE 33

### Carrier Linked Pramipexole Prodrug

### Synthesis of OEG-Linker Pramipexole Conjugate (33)

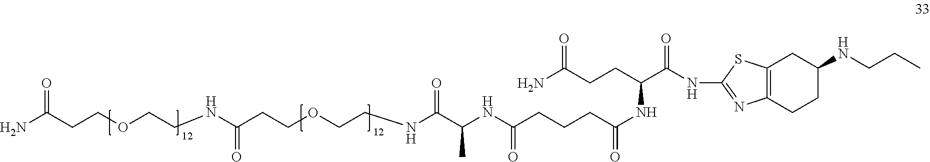

33 was synthesized on Sieber amide resin (89 mg, 0.056 mmol). All reactions were performed as described in “Materials and Methods”. In brief, the following steps were performed: 1. resin loading with Fmoc-PP-OH, 2. Fmoc-deprotection, 3. coupling of Fmoc-PP-OH, 4. Fmoc-deprotection, 5. coupling of Fmoc-Ala-OH, 6. Fmoc-deprotection, 7. coupling of 32c, 8. cleavage from resin. 33 was purified by RP-HPLC.

Yield: 50 mg (0.027 mmol).

MS: m/z 1723.0=[M+H]⁺ (calculated=1723.1 g/mol).

### EXAMPLE 34

### Synthesis of Linker Pramipexol Conjugate (34b)

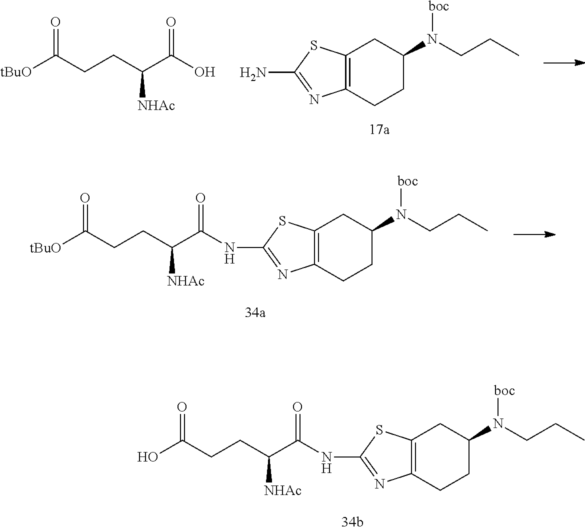

### Synthesis of Pramipexole Intermediate 34a

Intermediate 34a was synthesized by reacting boc-pramipexole 17a (150 mg, 0.482 mmol) with Ac-Glu(OtBu)-OH (142 mg, 0.578 mmol) by addition of PyBOP (376 mg, 0.722 mmol) and DIPEA (1684, 0.963 mmol) in DCM (2 mL). The solution was stirred for 3 h at RT. Volatiles were removed in vacuo and the product was purified by PR-HPLC.

Yield: 179 mg (0.333 mmol).

MS: m/z 539.3=[M+H]⁺ (calculated=538.7 g/mol).

### Synthesis of Pramipexole Intermediate 34b

For saponification of tBu ester 34a (179 mg, 0.33 mmol) was dissolved in 4 mL methanol. 3 mL water and 75 mg LiOH were added. Mixture was stirred for 2 h at 65° C. in a pressure vessel. 200 μl AcOH were added and product 34b was purified by RP-HPLC.

Yield: 142 mg (0.294 mmol).

MS: m/z 483.2=[M+H]⁺ (calculated=482.6 g/mol).

### EXAMPLE 35

### Carrier Linked Pramipexole Prodrug

### Synthesis of OEG-Linker Pramipexole Conjugate (35)

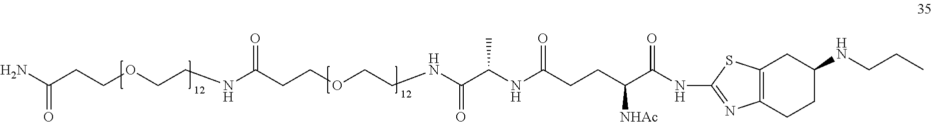

35 was synthesized similar to 33 except that 34b was used instead of 32c. 35 was purified by RP-HPLC.

Yield: 12.4 mg (0.008 mmol).

MS: m/z 1651.9=[M+H]⁺ (calculated=1652.0 g/mol).

### EXAMPLE 36

### Pramipexole Release Kinetics and Hydrogel Degradation Kinetics

Pramipexole linker hydrogel 30 was incubated in pH 7.4 buffer at 37° C. as described in Example 27. Hydrogel degradation over 150 days was monitored by SEC as described in “Materials and Methods”. Result: During lag phase of hydrogel degradation (60 days) approx. 90% of pramipexole is released due to matched pramipexole prodrug linker kinetics and hydrogel degradation kinetics. Hydrogel degradation was complete after 150 days, FIG. 2.

### EXAMPLE 37

### Single Dose Pharmacokinetics of Pramipexole

The pharmacokinetics of 30 were determined by measuring the plasma pramipexole concentration after subcutaneous application of a single dose into rats.

One group consisting of 5 male Wistar rats (200-250 g) was used to study the plasma pramipexole levels over a period of 28 days. Each of the animals received a single subcutaneous injection of 600 μL suspension of 30 in acetate buffer pH 5, containing 2.5 mg pramipexole. Per animal and time point 200 μL of blood was withdrawn sublingually to obtain 100 μL Li-Heparin plasma. Samples were collected before application and after 4 h, 1, 2, 3, 6, 8, 10, 14, 17, 21, 24 and 28 days postinjection. Plasma samples were frozen within 15 min after blood withdrawal and stored at −80° C. until assayed.

Averaged plasma pramipexole concentrations for each time point were obtained by calculating the mean of the 5 animals used.

A sustained release of pramipexole over 28 days without burst was observed after a single subcutaneous injection, FIG. 3.

### FIG. 3

### EXAMPLE 38

### Repeated Dose Pharmacokinetics of Pramipexole (P09.0209)

The pharmacokinetics of 26 were determined by measuring the plasma pramipexole concentration after subcutaneous application of three repeated doses into rats.

One group consisting of 5 male Wistar rats (200-250 g) was used to study the plasma pramipexole levels over a period of 38 days. At days 0, 7 and 14 each of the animals received a subcutaneous injection of 500 μA suspension of 26 in acetate buffer pH 5, containing 1.7 mg pramipexole. Per animal and time point 200 μL of blood was withdrawn sublingually to obtain 100 μl, Li-Heparin plasma. Samples were collected before application and after 4 h, 1 d; 4 d; 7 d; 7 d+4 h, 8 d, 11 d, 14 d, 14 d+4 h, 15 d, 18 d, 21 d, 24 d, 28 d, 31 d, 35 d and 38 d days postinjection. Plasma samples were frozen within 15 min after blood withdrawal and stored at −80° C. until assayed.

Averaged plasma pramipexole concentrations for each time point were obtained by calculating the mean of the 5 animals used.

A sustained release of pramipexole with low peak to trough ratios was observed after three repeated subcutaneous injections FIG. 4.

### FIG. 4

### EXAMPLE 39

### Single Dose Short Time Pharmacokinetics of Pramipexole (Burst Analysis) (P09.0256)

The pharmacokinetics of 31 were determined by measuring the plasma pramipexole concentration after subcutaneous application of a single dose into rats.

One group consisting of 3 male Wistar rats (200-250 g) was used to study the plasma pramipexole levels over a period of 24 hours. Each of the animals received a single subcutaneous injection of a suspension of 31 in acetate buffer pH 5. A dose of 6.3 mg pramipexole/kg (4.5 mg pramipexole/ml) was used. Per animal and time point 200 μL of blood was withdrawn sublingually to obtain 100 μL, Li-Heparin plasma. Samples were collected before application and after 5 min, 30 min, 90 min, 4 h and 24 h. Plasma samples were frozen within 15 min after blood withdrawal and stored at −80° C. until assayed. Averaged plasma pramipexole concentrations for each time point were obtained by calculating the mean of the 3 animals used.

A burstless release of pramipexole over 24 hours was observed after a single dose subcutaneous injection FIG. 5.

### FIG. 5

### EXAMPLE 40

### 30 G Needle Test

Hydrogel suspension 26 was washed ten times with formulation buffer (10 mM succinate buffer pH 5.0, 85 g trehalose dihydrate/L, 0.3% (weight) Pluronic F68). Hydrogel was left settling at 4° C. for 1 day and the supernatant was removed in order to obtain a dense suspension. The total volume of the dense suspension was determined (2.6 mL), and the suspension was lyophilized.

The lyophilization cake was reconstituted by addition of water to give a total volume of 2.6 mL. The suspension was homogenized by means of a vortexer. Suspension was drawn into two 1 mL Luer-lock syringes via a 20 G needle (0.9 mL each). The needle was exchanged to a 30 G needle. The suspension was ejected from one syringe manually. The suspension passed the needle without blocking.

The second syringe was stored horizontally at 4° C. for two weeks prior to syringeability test. Stored TransCon Hydrogel Pramipexole also passed the 30 G needle without blocking

### EXAMPLE 41

### Stability Test

Hydrogel suspension 26 was washed ten times with formulation buffer (10 mM succinate buffer pH 5.0, 85 g trehalose dihydrate/L, 0.3% (weight) Pluronic F68). Hydrogel was left settling at 4° C. for 1 day and the supernatant was removed in order to obtain a dense suspension. Aliquots of 34 μL TransCon Hydrogel Pramipexole suspension (containing 0.447 mg pramipexole each) were transferred in 1.5 mL Eppendorf tubes. Tubes were frozen in liquid nitrogen and lyophilized over night at RT and 0.05 mbar. Lyophilized Hydrogel Pramipexole samples were stored at 40° C.

At a given time aliquots were reconstituted by addition of water to obtain a total volume of 100 pt. Samples were vortexed and centrifuged. The supernatant was assayed for liberated pramipexole by SEC as described in “Materials and Methods”.

Result: After 7 months 0.22% pramipexole was liberated.

### ABBREVIATIONS

AcOH acetic acid
Ado 8-amino-3,6-dioxa-octanoic acid
Ava 5-aminovaleric acid
Boc t-butyloxycarbonyl
DBU 1,3-diazabicyclo[5.4.0]undecene
DCC dicyclohexylcarbodiimide
DCM dichloromethane
DIC diisopropyl carbodiimide
DIEA diisopropylethylamine
DMAP dimethylamino-pyridine
DMF N,N-dimethylformamide
DMSO dimethylsulfoxide
EDC 1-Ethyl-3-(3-dimethylaminopropyl)carbodiimid
EDTA ethylenediaminetetraacetic acid
EG ethylene glycol
ESI electrospray ionization
EtOH ethanol
eq stoichiometric equivalent
Fmoc 9-fluorenylmethoxycarbonyl
Fmoc-PP-OH Fmoc-aminoethyl-undecaethyleneoxide-propionic acid
HATU O-(7-Azabenzotriazol-1-yl)-N,N,N′,N′-tetramethyluronium hexafluorophosphate
HFIP hexafluoroisopropanol
HLB hydrophilic-lipophilic balance
HOBt N-hydroxybenzotriazole
LCMS mass spectrometry-coupled liquid chromatography
Mal 3-maleimido propionyl
Mal-dPEG₆-NHS N-(3-maleimidopropyl)-21-amino-4,7,10,13,16,19-hexaoxa-heneicosanoic acid NHS ester
MS mass spectrum/mass spectrometry
n. d. not determined
NHS N-hydroxy succinimide
OEG Oligo(ethylene glycol)
PEG poly(ethylene glycol)
PyBOP benzotriazole-1-yl-oxy-tris-pyrrolidino-phosphonium hexafluorophosphate
RP-HPLC reversed-phase high performance liquid chromatography
RT room temperature
TCP 2-chlorotrityl chloride resin
TES triethylsilane
TFA trifluoroacetic acid
THF tetrahydrofurane
TMEDA N,N,N′,N′, tetramethyl ethylene diamine
Trt trityl
HPLC ultra performance liquid chromatography
UV ultraviolet
VIS visual

1-41. (canceled)

42. A carrier linked pramipexole prodrug or a pharmaceutically acceptable salt thereof, wherein
pramipexole is bound via a linker L to a carrier and the carrier is a polymer having a molecular weight of ≧500 g/mol.

43. The prodrug according to claim 42, wherein pramipexole is bound to the carrier via the linker L by forming an amide bond with L.

44. The prodrug according to claim 42, wherein the linker L contains a moiety L¹ represented by formula (I), 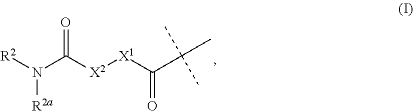
wherein the dashed line indicates the attachment of L¹ to pramipexole by forming an amide bond with the aromatic amino group of pramipexole;
X¹ is C(R¹R^{1a}) or a cyclic fragment selected from C₃₋₇ cycloalkyl, 4 to 7 membered heterocyclyl, phenyl, naphthyl, indenyl, indanyl, tetralinyl, or 9 to 11 membered heterobicyclyl;
X² is a chemical bond or selected from C(R³R^{3a}), N(R³), O, C(R³R^{3a})—C(R⁴R^{4a}), C(R³R^{3a})—N(R⁴), N(R³)—C(R⁴R^{4a}), C(R³R^{3a})—O, or O—C(R³R^{3a}),
wherein in case X¹ is a cyclic fragment, X² is a chemical bond, C(R³R^{3a}), N(R³) or O;
optionally, in case X¹ is a cyclic fragment and X² is C(R³R^{3a}), the order of the X¹ fragment and the X² fragment within L¹ may be changed;
R¹, R³ and R⁴ are independently selected from the group consisting of H, C₁₋₄ alkyl and —N(R⁵R^{5a});
R^{1a}, R², R^{2a}, R^{3a}, R^{4a} and R^{5a} are independently selected from the group consisting of H, and C₁₋₄ alkyl;
optionally, one of the pairs R^{2a}/R², R^{2a}/R^{3a}, R^{2a}/R^{4a} are joined to form a 4 to 7 membered at least partially saturated heterocycle;
R⁵ is C(O)R⁶;
R⁶ is C₁₋₄ alkyl;
optionally, one of the pairs R^{1a}/R^{4a}, R^{3a}/R^{4a} or R^{1a}/R^{3a} form a chemical bond;
optionally, L¹ is further substituted.

45. The prodrug of claim 44, wherein R^{1a}, R², R^{2a}, R^{3a}, R^{4a} and R^{5a} are independently selected from the group consisting of H, and C₁₋₄ alkyl.

46. The prodrug of claim 44, wherein R^{2a} is H and that hydrogen is not replaced by a substituent or represents a connection of L¹ to the carrier.

47. The prodrug according to claim 42, wherein the linker L contains a moiety L¹ represented by formula (II) 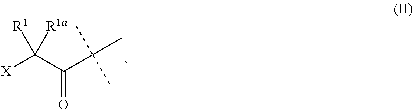
wherein the dashed line indicates the attachment of L¹ to pramipexole by forming, an amide bond with the aromatic amino group of pramipexole;
X is H or C₁₋₅₀ alkyl optionally interrupted by one or more groups selected from —NH—, —C(C₁₋₄ alkyl)₂-, —O—, —C(O)— or —C(O)NH—;
R¹ and R^{1a} are independently selected from the group consisting of H and C₁-C₄ alkyl;
optionally, L¹ is further substituted.

48. The prodrug of claim 47, wherein X in formula (II) includes one of the following fragments, wherein the dashed line on the right hand side indicates the attachment of L¹ to pramipexole by forming an amide bond with the aromatic amino group of pramipexole and the dashed line on the left hand side indicates the attachment to the rest of X and wherein L¹ is optionally further substituted:

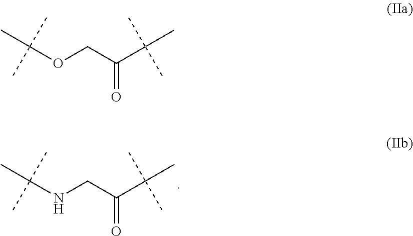

49. The prodrug of claim 48, wherein X in formula (II) includes one of the following fragments, wherein the dashed line on the right hand side indicates the attachment of L¹ to pramipexole by forming an amide bond with the aromatic amino group of pramipexole and the dashed line on the left hand side indicates the attachment to the rest of X:

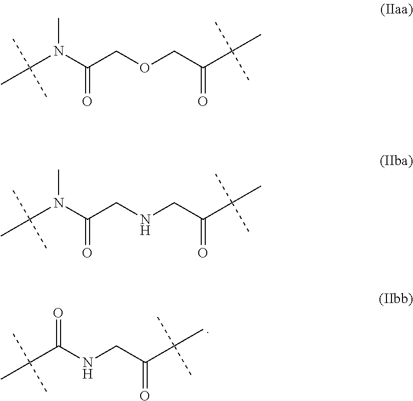

50. The prodrug according to claim 42, wherein the linker L contains a moiety L², which is a chemical bond or a spacer, and L² is bound to the carrier.

51. The prodrug according to claim 50, wherein the spacer is a fragment selected from C₁₋₅₀ alkyl, C₂₋₅₀ alkenyl or C₂₋₅₀ alkinyl, which fragment is optionally interrupted by one or more groups selected from —NH—, —N(C₁₋₄ alkyl)-, —O—, —S—, —C(O)—, —C(O)NH—, —C(O)N(C₁₋₄ alkyl)-, —O—C(O)—, —S(O)—, —S(O)₂—, 4 to 7 membered heterocyclyl, phenyl or naphthyl.

52. The prodrug according to claim 50, wherein L² is attached to the carrier via a terminal group selected from —CO—NH—,

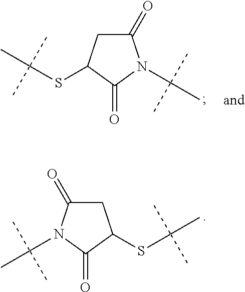

53. The prodrug according to claim 42, wherein the carrier is a polymer selected from 2-methacryloyl-oxyethyl phosphoyl cholins, hydrogels, PEG-based hydrogels, poly(acrylic acids), poly(acrylates), poly(acrylamides), poly(alkyloxy)polymers, poly(amides), poly(amidoamines), poly(amino acids), poly(anhydrides), poly(aspartamides), poly(butyric acids), poly(glycolic acids), polybutylene terephthalates, poly(caprolactones), poly(carbonates), poly(cyanoacrylates), poly(dimethylacrylamides), poly(esters), poly(ethylenes), poly(ethyleneglycols), poly(ethylene oxides), poly(ethyl phosphates), poly(ethyloxazolines), poly(glycolic acids), poly(hydroxyethyl acrylates), poly(hydroxyethyloxazolines), poly(hydroxymethacrylates), poly(hydroxypropylmethacrylamides), poly(hydroxypropyl methacrylates), poly(hydroxypropyloxazolines), poly(iminocarbonates), poly(lactic acids), poly(lactic-co-glycolic acids), poly(methacrylamides), poly(methacrylates), poly(methyloxazolines), poly(organophosphazenes), poly(ortho esters), poly(oxazolines), poly(propylene glycols), poly(siloxanes), poly(urethanes), poly(vinyl alcohols), poly(vinyl amines), poly(vinylmethylethers), poly(vinylpyrrolidones), silicones, celluloses, carbomethyl celluloses, hydroxypropyl methylcelluloses, chitins, chitosans, dextrans, dextrins, gelatins, hyaluronic acids and derivatives, mannans, pectins, rhamnogalacturonans, starches, hydroxyalkyl starches, hydroxyethyl starches and other carbohydrate-based polymers, xylans, and copolymers thereof.

54. The prodrug according to claim 42, wherein the carrier is a biodegradable poly(ethylene glycol) based water-insoluble hydrogel.

55. The prodrug of claim 54, wherein the hydrogel is composed of backbone moieties interconnected by hydrolytically degradable bonds.

56. The prodrug of claim 54, wherein the backbone moieties comprise a branching core of the following formula: 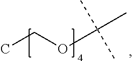
wherein the dashed line indicates attachment to the remainder of the backbone moiety.

57. The prodrug of claim 54, wherein the backbone moieties comprise a structure of the following formula: 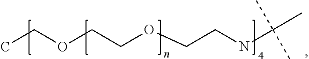
wherein n is an integer of from 5 to 50 and the dashed line indicates attachment to the rest of the molecule.

58. The prodrug of claim 54, wherein the backbone moiety comprises a hyperbranched moiety Hyp.

59. The prodrug of claim 58, wherein the backbone moiety comprises a hyperbranched moiety Hyp of the following formula: 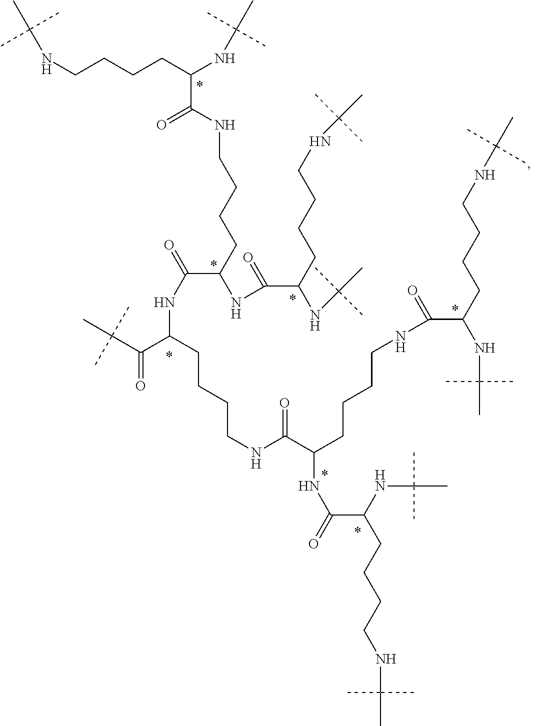
wherein the dashed lines indicate attachment to the rest of the molecule and carbon atoms marked with asterisks indicate S-configuration.

60. The prodrug of claim 55, wherein the backbone moieties are attached to at least one spacer of the following formula: 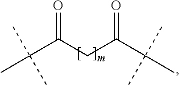
wherein one of the dashed lines indicates attachment to the hyperbranched moiety Hyp and the second dashed line indicates attachment to the rest of the molecule; and
wherein m is an integer of from 2 to 4.

61. The prodrug of claim 55, wherein the backbone moieties are linked together through crosslinker moieties comprising the following structure 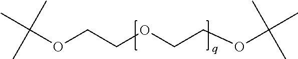
wherein
q is an integer from 3 to 100;

62. The prodrug of claim 54 in the form of microparticles.

63. The prodrug of claim 62, wherein the microparticles have a diameter of between 20 and 100 micrometer

64. The prodrug of claim 62, wherein the microparticles can be administered by injection through a needle smaller than 0.6 mm inner diameter.

65. The prodrug of claims 62, wherein the microparticles can be administered by injection through a needle smaller than 0.3 mm inner diameter.

66. The prodrug of claim 62, wherein the microparticles can be administered by injection through a needle smaller than 0.2 mm inner diameter.

67. A pharmaceutical composition comprising an effective dose of at least one prodrug or a pharmaceutically acceptable salt thereof according to claim 42 and a pharmaceutically acceptable excipient.

68. A pharmaceutical composition according to claim 67, which is an injectable slow release composition with an effective dose of 10 to 100 mg/mL, based on the quantitative release of free pramipexole, of at least one prodrug or a pharmaceutically acceptable salt thereof.

69. A pharmaceutical composition according to claim 67, wherein the pharmaceutical composition is dry.

70. A pharmaceutical composition according to claim 69, wherein the pharmaceutical composition was dried by lyophilization.

71. A pharmaceutical composition according to claim 67, wherein the pramipexole hydrogel prodrug is sufficiently dosed in the composition to provide a therapeutically effective amount of pramipexole for at least three days in one application.

72. A pharmaceutical composition according to claim 67, wherein it is a single dose composition.

73. A pharmaceutical composition according to claim 67, wherein it is a multiple dose composition.

74. A container comprising the pharmaceutical composition according to claim 67.

75. A container according to claim 74, wherein the container is a dual-chamber syringe.

76. A suspension comprising the pharmaceutical composition according to claim 67.

77. A method of preparing a suspension according to claim 76, comprising the steps of reconstituting the dry pharmaceutical composition according to claim 69 by adding reconstitution solution.

78. A kit of parts, comprising a needle and a container containing reconstitution solution and the dry composition according to claim 69 for use with the needle.

79. A kit of parts according to claim 78, wherein the container is a dual-chamber syringe and wherein one of the two chambers of the dual-chamber syringe contains the dry pharmaceutical composition and the second chamber of said dual-chamber syringe contains the reconstitution solution.

80. A method for prophylaxis and/or treatment of dopamine receptor related diseases, including Parkinson's disease, neurological disorders, amyotrophic lateral sclerosis, compulsive behavior, bipolar disorders, Tourette's syndrome, depressive disorders, treatment resistant depression, fibromyalia or restless leg syndrome (RLS), wherein a prodrug according to claim 42 is used.

81. A method for prophylaxis and/or treatment of dopamine receptor related diseases, including Parkinson's disease, neurological disorders, amyotrophic lateral sclerosis, compulsive behavior, bipolar disorders, Tourette's syndrome, depressive disorders, treatment resistant depression, fibromyalia or restless leg syndrome (RLS), wherein a pharmaceutical composition according to claim 67 is used.

82. A method for the synthesis of a prodrug or a pharmaceutically acceptable salt thereof according to claim 42 comprising the step of reacting a prodrug precursor L-Y or L1-Y with pramipexole to obtain a pramipexole linker conjugate by forming an amide bond, wherein Y is a leaving group and L or L1 are optionally bound to the carrier.
